(19) United States
(12) Patent Application Publication
(10) Pub. No.: US 2025/0169802 A1
(43) Pub. Date: May 29, 2025

(54) SYSTEMS AND METHODS OF ENHANCING TUMOR-REACTIVE IMMUNE POPULATIONS WITH ORGANOIDS

(71) Applicant: NEXTVIVO, INC., Palo Alto, CA (US)

(72) Inventors: Cécile Chartier-Courtaud, Palo Alto, CA (US); Marc Leushacke, Mountain View, CA (US); Jihang Ju, Fremont, CA (US); Lisiena Hysenaj, San Francisco, CA (US); Adam Margolin, Palo Alto, CA (US)

(21) Appl. No.: 18/962,995

(22) Filed: Nov. 27, 2024

Related U.S. Application Data
(63) Continuation-in-part of application No. PCT/US2023/032111, filed on Sep. 6, 2023.
(60) Provisional application No. 63/605,393, filed on Dec. 1, 2023.
(60) Provisional application No. 63/374,891, filed on Sep. 7, 2022.
(60) Provisional application No. 63/499,685, filed on May 2, 2023.

(51) Int. Cl. A61B 10/02; A61B 10/00

(52) U.S. Cl. CPC A61B 10/02; A61B 10/0096

(57) ABSTRACT

Provided are systems and methods of enhancing tumor-reactive immune populations such as tumor infiltrating lymphocytes from patient-derived organoids. Also, provided herein are systems and methods of expanding and reprogramming tumor-reactive immune populations such as tumor infiltrating lymphocytes to improve anti-tumor activity. The systems and methods described herein can be used for adoptive cell therapy.

### CROSS-REFERENCE

This application claims the benefit of U.S. Provisional Application No. 63/605,393, filed Dec. 1, 2023, and is a continuation-in-part of International Patent Application No. PCT/US2023/032111, filed Sep. 6, 2023, which itself claims the benefit of U.S. Provisional Application No. 63/374,891, filed Sep. 7, 2022, and U.S. Provisional Application No. 63/499,685, filed May 2, 2023, each of which is incorporated herein by reference in its entirety.

### BACKGROUND

Adoptive cellular therapy utilizes cells from immune systems, such as tumor-reactive immune cells, for cancer or tumor treatment. This form of immunotherapy obtains cells, e.g., tumor-reactive immune cells, from patients. These cells are then expanded or, in some instances, engineered or reprogrammed to enhance their ability to target and eliminate cancer cells. These cells are then given back to the patients for cancer treatment. The process of expanding tumor-reactive immune cells and the quality of tumor-reactive immune cells are still limited.

### SUMMARY

There is an unmet need for enhancing tumor-reactive immune cells for cancer and tumor treatment. This disclosure meets this unmet need.

In one aspect, the present disclosure provides a method of generating subject derived immune cells, the method comprising: (a) obtaining one or more tumor samples from a subject, wherein the one or more tumor samples comprise immune cells; (b) incubating the one or more tumor samples using an in vitro culture process, wherein the one or more tumor samples are not submerged in culture medium, wherein one or more agents are added to the culture medium; and (c) collecting the immune cells from the one or more tumor samples, thereby generating the subject derived immune cells.

In some embodiments, the incubating of (b) comprises a first time period and a second time period. In some embodiments, the first time period is at least about 1 day. In some embodiments, the first time period is about 7 to about 14 days. In some embodiments, the second time period is at least about 1 day. In some embodiments, the second time period is about 11 days.

In some embodiments, the one or more agents are added to the culture medium at a first concentration during the first time period. In some embodiments, the one or more agents are added to the culture medium at a second concentration during the second time period. In some embodiments, the first concentration is lower than the second concentration.

In some embodiments, the in vitro culture process comprises an air-liquid interface setup.

In some embodiments, the method further comprises, cryopreserving the immune cells from (c). In some embodiments, the method further comprises (d) expanding the immune cells obtained from (c) using one or more agents for a time period. In some embodiments, the method further comprises (e) collecting the immune cells from (d). In some embodiments, the method further comprises after (e), (f) cryopreserving the immune cells.

In some embodiments, the immune cells comprise tumor infiltrating lymphocytes. In some embodiments, the tumor infiltrating lymphocytes comprise T cells. In some embodiments, the T cells comprise activated T cells, naïve CD8+ T cells, cytotoxic CD8+ T cells, naïve CD4+ T cells, helper T cells, e.g. T_{H}1, T_{H}2, T_{H}9, T_{H}17, T_{H}22, T_{FH}; memory T cells, e.g. central memory T cells, T stem cell memory cells (TSCM), effector memory T cells, NKT cells, or γd T cells.

In some embodiments, the one or more agents in (b) comprises a cytokine. In some embodiments, the cytokine comprises IL-2, an IL-2 variant, IL-7, an IL-7 variant, IL-15, an IL-15 variant, IL-18, an IL-18 variant, IL-21, an IL-21 variant, or combination thereof. In some embodiments, the concentration of the cytokine added during the first time period in (b) is at least about 10 IU/mL. In some embodiments, the concentration of the cytokine added during the first time period in (b) is about 50 IU/mL. In some embodiments, the concentration of the cytokine added during the second time period in (b) is at least about 4000 IU/mL. In some embodiments, the concentration of the cytokine added during the second time period in (b) is about 6000 IU/mL.

In some embodiments, the expanding of the immune cells in (d) does not comprise an air-liquid interface setup. In some embodiments, the time period in (d) is at least 1 day. In some embodiments, the time period in (d) is about 14 days. In some embodiments, the one or more agents in (d) comprises a cytokine, an antibody, a modulator, or any combination thereof.

In some embodiments, the one or more agents further comprises irradiated feeder cells. In some embodiments, the irradiated feeder cells are irradiated allogeneic PBMC-derived feeder cells. In some embodiments, a ratio of the immune cells to the irradiated feeder cells is about 1:100.

In some embodiments, the cytokine comprises IL-2, an IL-2 variant, IL-7, an IL-7 variant, IL-15, an IL-15 variant, IL-18, an IL-18 variant, IL-21, an IL-21 variant, or any combination thereof. In some embodiments, the cytokine is added at a concentration of at least about 2000 IU/mL. In some embodiments, the cytokine is added at a concentration of about 3000 IU/mL.

In some embodiments, the antibody comprises an anti-CD3 antibody. In some embodiments, the concentration of the anti-CD3 antibody is at least about 10 ng/mL. In some embodiments, the concentration of the anti-CD3 antibody is about 10 ng/mL. In some embodiments, the concentration of the anti-CD3 antibody is about 30 ng/mL.

In some embodiments, the modulator comprises a Notch signaling pathway modulator, an interferon gamma (IFNγ) modulator, or a combination thereof. In some embodiments, the Notch signaling pathway modulator comprises a Notch activator. In some embodiments, the Notch activator comprises an antibody, a small molecule, or a combination thereof. In some embodiments, the IFNγ modulator comprises an IFNγ inhibitor. In some embodiments, the IFNγ inhibitor comprises an antibody, a small molecule, or a combination thereof.

In some embodiments, the method further comprises providing additional reagents during the first time period or the second time period, wherein the additional reagents comprise one or more of PD-1, CD39, 4-1BB-positive T cells, or CXCR3-binding chemokines. In some embodiments, the CXCR3-binding chemokines comprise CXCL9 or CXCL10.

In some embodiments, the method further comprises providing additional reagents during the first time period or the second time period, wherein the additional reagents comprise interferons.

In some embodiments, the method further comprises providing additional reagents during the first time period or the second time period, wherein the additional reagents comprise checkpoint inhibitors.

In some embodiments, the method further comprises providing additional reagents during the first time period or the second time period, wherein the additional reagents comprise TLR3, TLR7, TLR9, or other TLR agonists.

In some embodiments, the method further comprises providing additional reagents during the first time period or the second time period, wherein the additional reagents comprise modulators of RIG-I-like receptors, modulators of NOD-like receptors, modulators of C-type lectin receptors, modulators of STING, or combination thereof.

In some embodiments, the method further comprises combining tumor organoids with immune cells obtained from other sources. In some embodiments, the other sources comprise peripheral blood cells or organoids grown from lymphoid tissue.

In some embodiments, the method further comprises stimulating antigen presentation.

In some embodiments, the method further comprises depletion of immune inhibitory cell types. In some embodiments, the immune inhibitory cell types comprise Tregs, myeloid derived suppressor cells, TAMs, vascular endothelial cells, or CAFs.

In some embodiments, the method further comprises negative selection of bystander tumor reactive immune cells.

In some embodiments, the method further comprises knock-down exhaustion regulator. In some embodiments, the knock-down exhaustion regulator is TOX.

In some embodiments, the method further comprises reprogramming the immune cells. In some embodiments, the immune cells are reprogrammed by activation of Notch signaling pathway during (d), inhibition of interferon gamma (IFNγ) signaling pathway during (d), or both.

In some embodiments, the method further comprises identifying T cell receptor (TCR) to identify TCRs enriched in the air-liquid interface culture. In some embodiments, the identifying TCR is performed using sequencing technology.

In some embodiments, the method comprises providing one or more tumor antigens during the first time period or the second time period. In some embodiments, the providing of one or more tumor antigens comprises providing cells expressing the tumor antigens.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts an exemplary timeline for a pre-Rapid Expansion Protocol (pre-REP) step of a Standard Tumor Infiltrating Lymphocytes (STD TILs) protocol and of an air-liquid interface Tumor Infiltrating Lymphocytes (ALI TILs) protocol.

FIG. 2 shows a graph illustrating that co-culturing of air-liquid interface Tumor Infiltrating Lymphocytes (ALI TILs) with autologous tumor epithelial cells demonstrates a better tumor killing capacity compared to co-culturing of standard Tumor Infiltrating Lymphocytes (or STD TILs) with autologous tumor epithelial cells. The Y-axis represents % of live tumor epithelial cells.

FIGS. 3A-3C show that ALI TILs are more reactive to tumor cells compared to STD TILs. FIG. 3A shows an experimental outline. FIG. 3B and FIG. 3C show graphs illustrating that ALI Tumor Infiltrating Lymphocytes (ALI TILs) are more reactive to tumor cells compared to standard TILs (STD TILs) based on the percentage of CD3+ cells secreting IFNγ as shown in FIG. 3B, and percentage of CD3+ cells expressing CD107a as shown in FIG. 3C. Data is shown for samples derived from 3 different patients (CRC-1=colorectal cancer patient 1, CRC-2=colorectal cancer patient 2, MLN=melanoma patient). Three replicates per experiment were performed. Nonparametric ANOVA tested corrected by Geisser Greenhouse Correction was performed to compare between different conditions; *≤Pv=0.05.

FIGS. 4A-4D show that ALI TILs express higher levels of HLA-DR compared to STD TILs. FIG. 4A shows an experimental outline. FIG. 4B, FIG. 4C, and FIG. 4D show graphs illustrating the expression levels of PD1, HLA-DR, and CD137, respectively. FIG. 4C shows that ALI Tumor Infiltrating Lymphocytes (ALI TILs) express higher levels of HLA-DR compared to standard TILs (STD TILs). The result is presented as Fold Change. Data is shown for samples derived from 3 different patients (CRC-1=colorectal cancer patient 1, CRC-2=colorectal cancer patient 2, MLN=melanoma patient). Three replicates per experiment were performed. Nonparametric ANOVA tested corrected by Geisser Greenhouse Correction was performed to compare between different conditions; *≤Pv=0.05.

FIG. 5 shows that ALI Tumor Infiltrating Lymphocytes (ALI TILs) express higher percentage of HLA-DR+ compared to standard TILs (STD TILs). Data is shown for samples derived from 3 different patients (CRC-1=colorectal cancer patient 1, CRC-2=colorectal cancer patient 2, MLN=melanoma patient). Three replicates per experiment were performed. Nonparametric ANOVA tested corrected by Geisser Greenhouse Correction was performed to compare between different conditions; *≤Pv=0.05.

FIGS. 6A-6C illustrate an example of the establishment of ALI tumor organoids.

FIG. 6A shows an example of ALI tumor organoids generated from tissue obtained from kidney, lung, esophagus, and uterus. FIG. 6B shows an example of ALI tumor organoids generated from tissue obtained from colon and glioblastoma (GBM). Top figures show samples grown as ALI organoids. Bottom figures show submerged organoids. FIG. 6C shows sequencing results for three colorectal cancer (CRC) organoid lines, which are shown as CNV plots and mutated genes (APC, TP53, KRAS).

FIGS. 7A-7C demonstrate the generation and phenotypic characterization of ALI TILs. FIG. 7A depicts a schematic representation of the 2-step ALI TIL process and cryopreservation of the product. FIG. 7B shows cell number of ALI TILs obtained from 14 preps (left) and the fold-expansion distribution of the 14 products (right). FIG. 7C shows results of flow cytometry analysis using CD3, γδTCR, CD4, CD8, CD45RA, and CD62L to detect T cells, lineage, and memory subsets. Results are plotted as percent parent for each individual sample with average and SEM.

FIGS. 8A and 8B show ALI TIL tumor reactivity and cytotoxicity detection. FIG. 8A shows flow cytometry analysis of ALI TILs from CRC co-cultured with autologous tumor organoids. Increased IFNγ, CD107a, and 4-1BB (or CD137) were observed. FIG. 8B shows results obtained from confocal imaging at 0 hour (0h) and 24 hours (24h) of co-culturing experiment of ALI TILs and autologous tumor organoids. Tumor cell killing was observed over time.

FIGS. 9A and 9B display the results of single-cell RNA seq analysis of CRC and melanoma ALI-TILs. FIG. 9A shows numbers of unique clonotypes relative to number of sequenced cells (upper row) and distribution of clonotype frequencies (lower row). FIG. 9B shows expression levels of 397 immune genes which identify 10 clusters using SeqGeq.

FIGS. 10A-10E show clinically relevant numbers of T cells generated from an ALI TIL protocol described herein.

FIGS. 11A-11E show phenotypic characterization of ALI TILs, generated by the methods provided herein.

FIGS. 12A and 12B show CRC submerged organoid characterization.

FIGS. 13A-13D show detection of ALI-TIL cytotoxicity in CRC organoids.

FIGS. 14A-14D show assessment of ALI-TIL in vivo efficacy.

FIGS. 15A-15E show assessment of ALI-TIL in vivo distribution.

### DETAILED DESCRIPTION

Adoptive cellular immunotherapy utilizes a patient's own immune cells for cancer treatment. The immune cells are isolated from a patient's own blood or tumor tissue, grown and expanded in the laboratory, and then given back to the patient to treat the patient's cancer. In some instances, the immune cells are engineered to enhance their ability to target cancer cells, e.g., as in chimeric antigen receptor (CAR) T cell therapies.

Tumor Infiltrating Lymphocytes are another type of adoptive cellular immunotherapy that has been developed for treating solid tumors. Lymphocytes or white blood cells, e.g., T cells or B cells, are parts of the immune system that help the body fight infections and eliminate abnormal cells, e.g., cancer or tumor. Once lymphocytes recognize abnormal cells and penetrate into the tumor, these cells are called Tumor Infiltrating Lymphocytes (TILs or TIL). TILs are known to kill cancer cells.

One of the main benefits of using TILs is that because TILs are obtained directly from the tumor, they recognize targets on the cancer cells. Further, it is possible to obtain a group of TILs from the tumor which can recognize multiple unique targets on the cancer cells in order to anticipate and prevent the tumor from adapting to the therapy.

In order to use TILs effectively as a cellular therapy for cancer or tumor treatment, TILs are first collected from the tumor during a biopsy. Expansion of TILs in vitro is next performed in order to obtain a large population of these immune cells suitable for administration to the patient.

The present disclosure provides systems and methods for enhancing tumor-reactive immune populations using patient derived organoids (PDOs). There are two main steps of enhancing and expanding tumor-reactive immune cells: pre-Rapid Expansion Protocol (or pre-REP) step and Rapid Expansion Protocol (REP) step. In standard (STD) protocol, the pre-REP step utilizes one concentration of IL-2 treatment throughout a time period. In contrast, in this present disclosure, the method of pre-REP step discloses an additional time period (e.g., an additional week) of low concentration of IL-2 treatment as compared to standard protocol. TILs obtained from PDOs (also called “air-liquid interface TIL” or “ALI TIL”) using the protocol described herein demonstrate better tumor killing capacity and are more reactive to tumor cells when compared to TILs obtained from the standard protocol. Thus, ALI TILs obtained from this present disclosure may provide effective immune cells for cancer immunotherapy, e.g., adoptive cellular therapy. Further, the present disclosure provides methods for reprogramming TILs to stem memory immune cells at the REP step by inhibiting IFNγ signaling pathway and activating Notch signaling pathway. The stem cell-like properties of the reprogrammed immune cells provide improved lifespan, self-renewal capacity, and effector differentiation potential, towards generating anti-tumor or anti-cancer TILs with augmented anti-tumor or anti-cancer activity.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be human, primate, a non-human primate, equine, bovine, porcine, canine, feline, rodent, e.g. mice, rats, hamster, etc. The subject may include, but is not limited to, human, cow, dog, mouse, rat, rabbit, guinea pig, chicken, fish, bird, reptile, camelid, bovine, chimpanzee, sheep, goat, and non-human primate. The subject may be diagnosed or suspected of being at high risk for a disease. In some cases, the subject is not necessarily diagnosed or suspected of being at high risk for the disease.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the term “immune cell” refers to cells that are of hematopoietic origin and that play a role in the immune response. In some instances, the immune cells comprise lymphocytes, such as B cells and T cells; natural killer cells; dendritic cells; myeloid cells, such as monocytes, macrophages, eosinophils, mast cells, basophils, and granulocytes. In some embodiments, the immune cells comprise a complex mixture of immune cells, e.g., tumor infiltrating lymphocytes (TILs) isolated from an individual in need of treatment.

As used herein, the term “T cells” refers to mammalian immune effector cells that may be characterized by expression of CD3 and/or T cell antigen receptor. In some embodiments, the T cells comprise naïve CD8+ T cells, cytotoxic CD8+ T cells, naïve CD4+ T cells, helper T cells, e.g. T_{H}1, T_{H}2, T_{H}9, T_{H}17, T_{H}22, T_{FH}; memory T cells, e.g. central memory T cells, T stem cell memory cells (T_{SCM}), effector memory T cells, NKT cells, γδ T cells.

As used herein, the term “adoptive cellular therapy” refers to a type of immunotherapy that utilizes patient's own immune cells, e.g., T cells or B cells, to help the body fight diseases, e.g., cancer or tumor. In some instances, adoptive cellular therapy is also known as adoptive cell transfer, cellular adoptive immunotherapy, or T-cell transfer therapy.

As used herein, the terms “cancer” or “tumor” are used interchangeably to refer to cells which exhibit autonomous, unregulated growth, such that they exhibit an abnormal growth phenotype characterized by a significant loss of control over cell proliferation. Cells or tissues of interest for detection, analysis, or treatment in the present disclosure comprise precancerous (e.g., benign), malignant, pre-metastatic, metastatic, and non-metastatic cells. Cancers of virtually every tissue are known. Further, cancer is not limited to any stage, grade, histomorphological feature, invasiveness, aggressiveness or malignancy of an affected tissue or cell aggregation. Different stages/grade/malignancy of cancer comprises stage 0 cancer, stage I cancer, stage II cancer, stage III cancer, stage IV cancer, grade I cancer, grade II cancer, grade III cancer, malignant cancer and primary carcinomas. As used herein, the term “cancer cell” refers to a cancer cell or is derived from a cancer cell e.g. clone of a cancer cell. Many types of cancers are known to those of skill in the art, including solid tumors such as carcinomas, sarcomas, glioblastomas, melanomas, lymphomas, myelomas, etc. Examples of cancer comprise ovarian cancer, breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, esophageal cancer, uterine cancer, and brain cancer.

As used herein, the terms “tumor infiltrating lymphocytes” or “tumor infiltrating lymphocyte” or “TILs” or “TIL” are used interchangeably to refer to immune cells, e.g., lymphocytes, that are found in tumor. In some embodiments, TILs can recognize and destroy cancer cells. In some embodiments, TILs are obtained from patient's tumor. In some embodiments, TILs are obtained from patient-derived tumor sample. In some embodiments, TILs are obtained from patient-derived organoids. Examples of TILs include, but are not limited to, T lymphocytes, e.g., CD4+ or CD8+ T-cells, B lymphocytes, macrophages, or NK cells.

As used therein, the terms “cell culture” or “culture” refer to the maintenance of cells in an artificial, in vitro environment. In some instances, the term “cell culture” is a generic term and may be used to encompass the cultivation not only of individual cells, but also of tissues or organs or samples that are parts of tissues or organs and are derived from a patient. As used herein, the term “culture system” is used herein to refer to the culture conditions in which the cells, tissues, organs, or samples that are derived from tissues or organs of a patient are grown. This culture system promotes prolonged tissue or cell expansion with proliferation, multilineage differentiation and recapitulation of cellular and tissue ultrastructure. In some instances, a culture system refers to an air-liquid interface (ALI) 3D culture system. In some instances, a culture system also refers to a non-ALI culture in which cells of interest can be expanded, e.g. on feeder layer cells.

As used herein, the terms “air-liquid interface” or “ALI” refer to the interface to which the tumor cells, tissues, or samples derived from a patient's tissue or organ are exposed to in the cultures described herein. In some instances, the primary tissue may be mixed with a gel solution which is then poured over a layer of gel formed in a container with a lower semi-permeable support, e.g., a membrane. In some instances, the container is placed in an outer container that contains the medium such that the gel containing the tissue in not submerged in the medium. In some instances, the gel solution containing the primary tissue is exposed to air from the top and to liquid medium from the bottom.

As used herein, the term “container” refers to a glass, plastic, or metal vessel that can provide an aseptic environment for culturing cells.

As used herein, the term “organoid” refers to a 3-dimensional growth of tumor tissue in culture that retains characteristics of the tumor in vivo. In some instances, organoid recapitulates cellular and tissue ultrastructure, immune cell interactions, etc. In some instances, organoids for use in the methods described herein are generally cultured from a tumor biopsy section. In some instances, the organoids may be generated using any method known in the art, depending on the application. Methods of organoid culture as described in the present disclosure, comprise a submerged method, an air-liquid interface method, a droplet and bioreactor method, etc.

As used herein, the term “gel substrate” refers to conventional meaning of a semi-solid extracellular matrix. Gel described here in includes, but is not limited to, collagen gel, matrigel, extracellular matrix proteins, fibronectin, collagen in various combinations with one or more of laminin, entactin (nidogen), fibronectin, and heparin sulfate; or human placental extracellular matrix.

As used herein, the term “ultrastructure” refers to the three-dimensional structure of a cell or tissue observed in vivo. Examples of ultrastructure include, but is not limited to, the ultrastructure of a cell which can be its polarity or its morphology in vivo, or the ultrastructure of a tissue which can be the arrangement of different cell types relative to one another within a tissue.

As used herein, the term “biological sample” refers to liquid samples of biological origin (e.g., blood, sputum, semen, mucus, urine, cerebrospinal fluid), solid tissue samples such as a biopsy specimen or tissue cultures or cells derived therefrom and the progeny thereof for example clinical samples or tissue obtained by surgical resection, tissue obtained by biopsy, cells in culture, cell supernatants, cell lysates, tissue samples, organs, bone marrow, blood, plasma, serum, and the like. A “biological sample” comprises a sample obtained from a patient's cancer cell, e.g., a sample comprising polynucleotides and/or polypeptides that is obtained from a patient's cancer cell (e.g., a cell lysate or other cell extract comprising polynucleotides and/or polypeptides); and a sample comprising cancer cells from a patient. A biological sample comprises a cancer cell from a patient and can also comprise non-cancerous cells. In some instances, samples have been manipulated in any way after their procurement, such as by treatment with reagents; washed; or enrichment for certain cell populations. The term sample also comprises sample that have been enriched for particular types of molecules, e.g., nucleic acids, polypeptides, etc.

As used herein, the terms “air-liquid interface immune cells” or “ALI immune cells” are used interchangeably to refer to in vitro isolated immune cells obtained from systems and methods described herein. These immune cells are obtained from PDO culture derived from one or more tumor samples from a subject and are undergone activation and expansion using systems and methods described herein. In some instances, “immune cell” refers to cells that are of hematopoietic origin and that play a role in the immune response. In some instances, the immune cells comprise lymphocytes, such as B cells and T cells; natural killer cells; dendritic cells; myeloid cells, such as monocytes, macrophages, eosinophils, mast cells, basophils, and granulocytes. In some embodiments, the immune cells comprise a complex mixture of immune cells, e.g., tumor infiltrating lymphocytes (TILs) isolated from an individual in need of treatment. In some instances, “air-liquid interface immune cells” refers to “air-liquid interface tumor infiltrating lymphocytes”, “ALI TILs”, or “ALI TIL”.

As used herein, the terms “treatment” or “treating” are used in reference to a pharmaceutical or other intervention regimen for obtaining beneficial or desired results in the recipient. Beneficial or desired results include, but are not limited to, a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect comprises delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

### Immunotherapy

Immunotherapy utilizes immune cells to defend the body from infection and disease. There are several types of cancer immunotherapy. For example, immune checkpoint therapy utilizes immune cells, e.g., TILs, along with immune checkpoint inhibitors that block a negative immune checkpoint protein, e.g., CTLA-4, PD-1L, or PD-1, to allow T cells to continue working and to eliminate cancer cells. Cancer vaccines stimulate immune cells to recognize and destroy cancer cells. Monoclonal antibodies can attach to specific proteins on the cancer cell surface or on immune cells and increase the ability of immune cells to fight cancer. Cytokine therapy utilizes cytokines, e.g., interferons and/or interleukins to trigger an immune response to fight cancer. Adoptive cellular therapy utilizes patient's own immune cells to fight cancer after in vitro expansion or modification.

Adoptive cellular therapy focuses on increasing the number and/or improving the effectiveness of immune cells. In adoptive cellular therapy, patient's immune cells are isolated, expanded or modified in vitro, and then infused back into the patient to help the immune system fight cancer. For example, Chimeric Antigen Receptor (CAR) T cell therapy modifies T cells so that they are better at recognizing and attacking cancer cells. Similarly, Chimeric Antigen Receptor (CAR) natural killer (NK) cell therapy modifies NK cells instead of T cells to fight cancer. Some T cell therapies obtain tumor reactive T cells from blood and selects only those that recognize specific signatures of the cancer cells. These cells are then expanded before being given back to the patient. Tumor Infiltrating Lymphocyte (TIL) therapy utilizes patient's lymphocytes that are isolated from a tumor, expanded in vitro, and then given back to the patient.

Adoptive cellular therapy involves obtaining immune cells, e.g., TILs, from a patient's tumor. In some instances, the tumor can be obtained from the patient via biopsy or surgical resection. In some instances, the patient's tumor is derived and cultured as an organoid, e.g., patient derived organoids (PDOs) in an in vitro environment. Once the immune cells, e.g., TILs, are obtained, either from PDOs or directly from the tumor, and cultured in vitro, these immune cells, e.g., TILs, are then cultured in a pre-Rapid Expansion Protocol (pre-REP) step before being expanded in a Rapid Expansion Protocol step by T cell receptor (TCR) engagement and cytokine treatment. These immune cells, e.g., TILs, are then collected for experiments or for treatment.

In one aspect, the present disclosure provides systems and methods for enhancing tumor-reactive immune populations using patient derived organoids (PDOs). In another aspect, in the present disclosure, the methods provided herein involve a pre-Rapid Expansion Protocol or pre-REP step comprising an additional time period (e.g., a week) of low concentration IL-2 treatment. Immune cells, e.g., TILs, obtained from PDOs (also called “air-liquid interface immune cells, e.g., TILs,” or “ALI immune cells, e.g., TILs”) utilized in this protocol are shown to have a better tumor killing capacity and are more reactive to tumor cells when compared to immune cells, e.g., TILs, obtained from the standard protocol. Thus, ALI immune cells, e.g., ALI TILs, obtained from this present disclosure provides are more effective for cancer immunotherapy than TILs obtained from a standard protocol (e.g., without the additional time period of low concentration IL-2 treatment). Further, the present disclosure provides systems and methods for reprogramming TILs, e.g., obtained from PDOs, to have stem cell-like properties. In some instances, the reprogramming of TILs can be performed at the REP step.

In some embodiments, the systems and methods described herein are improved as compared to standard methods of generating or expanding immune cells, e.g., TILs. In some embodiments, the systems and methods described herein for generating and/or expanding immune cells, e.g., TILs, provide an improvement for immunotherapy. In some embodiments, the immune cells, e.g., TILs, obtained from the present disclosure provide an improvement of tumor killing capacity and reactivity. In some embodiments, the immune cells, e.g., TILs, obtained from the present disclosure are also reprogrammed into stem memory immune cell phenotypes, which have stem-cell like properties, such as improved lifespan, self-renewal capacity, and effector differential potential, thereby augmenting anti-tumor activity.

In some instances, tissues used in the present disclosure comprise adrenal cortical cancer, anal cancer, aplastic anemia, bile duct cancer, bladder cancer, bone cancer, bone metastasis, brain cancers, central nervous system (CNS) cancers, glioblastoma (GBM), peripheral nervous system (PNS) cancers, breast cancer, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, Hodgkin's lymphoma, Kaposi's sarcoma, kidney cancer, laryngeal and hypopharyngeal cancer, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcomas, melanoma skin cancer, non-melanoma skin cancers, stomach cancer, testicular cancer, thymus cancer, thyroid cancer, uterine cancer (e.g. uterine sarcoma), transitional cell carcinoma, vaginal cancer, vulvar cancer, mesothelioma, squamous cell or epidermoid carcinoma, bronchial adenoma, choriocarcinoma, head and neck cancers, teratocarcinoma, or Waldenstrom's macroglobulinemia tissue.

In some instances, the systems and methods provide immune cells, e.g., TILs, for adoptive cellular therapy. In some instances, the immune cells, e.g., TILs, obtained from the systems and methods described herein are used for adoptive cellular therapy alone. In some instances, the immune cells, e.g., TILs, obtained from the systems and methods described herein are used in combination with other immunotherapies, e.g., immune checkpoint therapy, monoclonal antibodies therapy, cytokine therapy, etc. In some instances, the immune cells, e.g., TILs, obtained from the systems and methods described herein are used in combination with chemotherapy. In some instances, the immune cells, e.g., TILs, obtained from the systems and methods described herein are used in combination with other cancer therapies, e.g., drug therapy.

### Patient-Derived Organoids (PDOS)

Patient-derived organoids (PDOs) are 3-dimensional in vitro cultures that recapitulate the cellular architecture and ultrastructure of the tumor or tissue samples from which they are derived from. In some embodiments, PDOs comprise immune cells, e.g., tumor infiltrating lymphocytes, parenchymal element, stromal element, epithelial cells, etc., associated with the tumor or tissue in vivo. In some embodiments, PDOs are cultured in an air-liquid interface (ALI) system. In some embodiments, PDO can be used for drug screening assay, in vitro disease modeling, or generation and/or expansion of immune cells, e.g., TILs, for immunotherapies, e.g., adoptive cellular therapy.

The air-liquid interface (ALI) method provides a culture system that allows the propagation of organoids both with epithelial and stromal components of tumors. In some embodiments, the ALI method utilizes Boyden chambers (cell culture inserts) popularly used for cell migration assays. In some embodiments, cells are embedded in extracellular matrix (ECM) gels in an upper surface of the cell culture inserts with a porous membrane underneath, which substantially increases the oxygen supply to the cells compared to an epithelial-only submerged organoid method. In some instances, cells obtain nutrients and growth factors from the medium placed in the outer dish through diffusion across the porous membrane on the lower surface. The ALI method provides many benefits for organoid culture—ALI method not only maintains stromal and immune cells from the tissue samples, but this system also retains the tumor microenvironment for an extended period of time. Example of ALI method to culture PDOs is described, for example, as in Neal et al. Cell. 2018 Dec. 13; 175(7):1972-1988.e16 which is incorporated herein in its entirety by reference.

In some instances, alternative organoid cultures can be used. In some instances, as in the alternative organoid culture, e.g., the droplet and bioreactor method, tissue is embedded into droplets of cell culture matrix, e.g., basement membrane extract (BME) or ECM gel, and then transferred into spinning bioreactors. In some instances, the continuous agitation provides improved absorption of nutrients and oxygen.

### A. Method to Generate PDO Cultures

In one aspect, the present disclosure provides culture systems and methods for the generation and expansion of tumor-specific immune cells by organoid culture of solid tumors, including stromal and immune cells associated with the tumors in vivo, to activate and expand TIL cells, e.g. T cells, specific for the tumor-associated antigens. In some embodiments, the PDO culture and method described herein comprises tumor cells, immune cells, etc., associated with the tumor or tissue in vivo. In some embodiments, the PDO culture and method described herein recapitulate the cellular architecture and ultrastructure of the tumor or tissue samples from which they are derived from.

In some embodiments, a biological sample is obtained to generate a PDO culture. In some embodiments, the biological sample comprises a tumor tissue sample. In some embodiments, the tumor tissue samples can be obtained by any convenient method, e.g., by biopsy, e.g. during endoscopy, during surgery, by needle, etc. In some embodiments, a septic technique is utilized to obtain the tumor tissue samples. In some embodiments, the tumor tissue samples comprise human tissue, particularly cancer and other lesions, e.g., solid tumor microbiopsy samples such as needle or fine needle aspirate. In some embodiments, the tumor tissue samples are taken at a single timepoint. In some embodiments, the tumor tissue samples are taken at multiple timepoints. In some embodiments, the tumor tissue samples can be as small as 10⁷ cells, 10⁶ cells, 10⁵ cells, or less. In some embodiments, the tumor tissue samples can be a tumor biopsy section of from about 0.1 mm², about 1 mm², about 10 mm², etc.

After the tissue sample is removed, the tissue is immersed in ice-cold buffered solution, e.g., PBS, Ham's F12, MEM, culture medium, etc. In some embodiments, pieces of tissue can be minced to a size less than about 1 mm\hat{ }3, and can be less than about 0.5 mm\hat{ }3, or less than about 0.1 mm\hat{ }3. The minced tissue is mixed with a gel substrate, e.g., a collagen gel solution, e.g. Cellmatrix type I-A collagen (Nitta Gelatin Inc.); a matrigel solution, etc. Next, the tissue-containing gel substrate is layered over a layer of gel (a “foundation layer”) in a container with a lower semi-permeable support, e.g., a membrane, supporting the foundation gel layer, and the tissue-containing gel substrate is allowed to solidify. This container is placed into an outer container containing a suitable medium, for example HAMs F-12 medium supplemented with fetal calf serum (FCS) at a concentration of from about 1 to about 25%, usually from about 5 to about 20%, etc. In some instances, an alternative serum to FCS can be used in the methods described herein. In some instances, parts of tumor tissue samples are preserved for different applications or experiments, e.g., histology or sequencing. In some embodiments, parts of tumor tissue samples are frozen for different applications or experiments. Various methods known in the art can be used to preserve tumor tissue samples.

The arrangement of the PDO culture as described above allows nutrients to travel from the bottom, through the membrane and the foundation gel layer to the gel layer containing the tissue In some embodiments, the level of the medium is maintained such that the top part of the gel, e.g., the gel layer containing the tissue sample, is not submerged in liquid but is exposed to air. As a result, the tissue is grown in a gel with an air-liquid interface environment. Example of an air-liquid interface culture system is described, for example, as in Ootani et al. in Nat Med. 2009 Jun;15(6):701-6, which is incorporated herein in its entirety by reference. In some embodiments, the air-liquid interface organoid cultures can be moved into other formats such as multi-wells for screening or in submerged 2D or 3D geometries where the cells are placed underneath the tissue culture medium.

In some embodiments, the PDO cultures can be maintained for up to 5 days, up to 7 days, up to 10 days, up to 15 days, up to 21 days, up to 28 days, or more. In some embodiments, tissue, e.g., primary tissue, is obtained from a solid tumor. In some instances, one or more tumor samples are obtained from a subject to generate PDO culture. The tumor tissue may be from any mammalian species, e.g., human, non-human primates, equine, bovine, porcine, canine, feline, rodent, e.g. mice, rats, hamster, etc. In some instances, the subject includes, but is not limited to human, cow, dog, mouse, rat, rabbit, guinea pig, chicken, fish, bird, reptile, camelid, bovine, chimpanzee, sheep, goat, and non-human primate. In some instances, the subject is human. In some embodiments, the PDO cultures can be passaged for maintenance. In some embodiments, the PDO cultures can be cryopreserved. In some embodiments, the PDO cultures can be cryopreserved and then cryorecovered for downstream applications.

In some embodiments, the PDO cultures described herein comprise cognate immune cells, e.g., endogenous immune cells present in the biopsy sample; allogeneic T cells; etc. Immune cells that can be cultured with PDOs include, but are not limited to, T cells, macrophages, B cells, natural killer cells (NK cells), etc., including any of the T cell subsets.

In some instances, the PDO cultures can comprise exogenous agents that are added to activate T cells that are present in the culture. In some embodiments, agents that activate T cells can be added to the culture. In some embodiments, the agents that activate T cells comprises immune checkpoint inhibitors, e.g., agents such as antibodies that inhibit the activity of CTLA4 (Cytotoxic T-Lymphocyte-Associated protein 4, CD152), PD1 (also known as PD-1; Programmed Death 1 receptor), PD-L1, PD-L2, LAG-3 (Lymphocyte Activation Gene-3), OX40, A2AR (Adenosine A2A receptor), B7-H3 (CD276), B7-H4 (VTCN1), BTLA (B and T Lymphocyte Attenuator, CD272), IDO (Indoleamine 2,3-dioxygenase), KIR (Killer-cell Immunoglobulin-like Receptor), TIM 3 (T cell Immunoglobulin domain and Mucin domain 3), VISTA (V-domain Ig suppressor of T cell activation), IL-2R (interleukin-2 receptor), T cell immunoreceptor with immunoglobulin and ITIM domain (TIGIT), cytokines, e.g., IL-2, an IL-2 variant, IL-7, an IL-7 variant, IL-15, an IL-15 variant, IL-18, an IL-18 variant, IL-21,an IL-21 variant, etc Variants of cytokine can comprise one or more mutations in the amino acid sequence compared to wild-type sequence. Variants of cytokine can be used to modulate a target signaling pathway. Examples of variants of cytokine include, but are not limited to, mutated variant or truncated variant. In some embodiments, a combination of agents that activate T cells are added to cultures. Combinations of agents can comprise a combination of two or more of any of the agents listed above. In some embodiments, activation of T cells comprises protocols to reverse T cell exhaustion, e.g. pulsatile stimulation, addition of kinase inhibitors such as dasatinib, etc.

In some instances, when PDOs are cultured with agents that activate T cells, the culture can be maintained for any period of time to activate T cells. In some instances, culturing time with agents that activate T cells can be for up to 2 days, up to 3 days, up to 4 days, up to 5 days, up to 6 days, up to 7 days, up to 8 days, up to 9 days, up to 10 days, up to 11 days, up to 12 days, up to 13 days, up to 14 days, up to 15 days, or more than 15 days. In some instances, culturing time with agents that activate T cells can be at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, or at least 30 days. Following culturing with one or more T cell activating agents, T cell activation can be assessed. Activated T cells can be identified and quantitated based on a number of criteria, e.g., expression of CD3, CD25, CD69, CD137, CD107a, Granzyme B (GZMB), Perform 1 (PRF1), PD1, etc. In some instances, activated T cells can be isolated based on expression of these activation markers. In some instances, non-activated PDO cultures, e.g., cultures that are not treated with a T cell activation agent, can be used as a control.

In some instances, the PDO culture is an ALI culture. In some instances, PDOs or ALI organoids can be cryopreserved. In some instances, PDOs or ALI organoids can be cryorecovered. In some instances, PDOs or ALI organoids can be passaged to maintain organoid culture. In some instances, the PDO cultures provide the immune cells comprising a complex mixture of immune cells, e.g., tumor infiltrating lymphocytes (TILs), that can further be expanded and later used for an individual in need of treatment such as in adoptive cell therapy. In some embodiments, the immune cells, e.g., TILs, comprises T cells.

### Expansion of Air-Liquid Interface Tumor Infiltrating Lymphocytes (Ali Tils)

Once the PDO cultures (also called ALI culture in the present disclosure) are established, these PDOs can be utilized to expand immune cells such as Tumor Infiltrating Lymphocytes (TILs) using Rapid Expansion Protocol (REP). Prior to expansion of cells in the REP step, immune cells from the ALI culture are first maintained in the pre-Rapid Expansion Protocol (pre-REP) step. In this pre-REP step, ALI derived immune cells are treated with cytokines, e.g., IL-2, to activate these immune cells, e.g., TIL. In some instances, during the pre-REP step, ALI derived immune cells are treated with two different concentrations of cytokines during two different time periods. In some instances, the first concentration of cytokines used during the first time period in the pre-REP step is lower than the second concentration of cytokines used during the second time period in the pre-REP step.

After the pre-REP step, ALI derived immune cells are subjected to a REP step. In this REP step, immune cells undergo rapid expansion, resulting in a large population of immune cells, e.g., TILs. In some instances, in this REP step, ALI derived immune cells are cultured with a cytokine, e.g., IL-2, an antibody, e.g., anti-CD3 antibody, and feeder cells, e.g., PBMC-derived feeder cells. These immune cells are cultured for a period of time until a large population of immune cells are achieved. These immune cells can then be harvested and used for immunotherapy or for other applications. In some embodiments, the PDO-derived TILs or ALI-derived TILs are further reprogrammed to have stem cell-like properties, such as improved lifespan, self-renewal capacity, and effector differentiation potential, thereby augmenting anti-tumor activity of these cells. In some embodiments, these immune cells, e.g., TILs are reprogrammed into stem cell-like properties during the REP step, such as by activating Notch signaling pathway, inhibiting interferon gamma (IFNγ) signaling pathway, or a combination of both. In some embodiments, the immune cells, e.g., TILs, comprise T cells.

B. Pre-Rapid Expansion Protocol (pre-REP)

Activation of immune cells, e.g., TILs, occurs in a pre-REP step, and this process trains immune cells, e.g., TILs, to be more effective at expansion during REP step and better at targeting tumor cells.

In one aspect, the present disclosure provides a method of generating air-liquid interface (ALI) derived immune cells, wherein the method comprises (a) obtaining one or more tumor samples from a subject, wherein the one or more tumor samples comprise immune cells; (b) incubating the one or more tumor samples using in vitro culture process, wherein the one or more tumor samples are not submerged in culture medium, wherein one or more agents are added to the culture medium; and (c) collecting the immune cells from the one or more tumor samples. In some embodiments, incubating the one or more tumor samples using an in vitro culture process comprises a first time period and a second time period. In some embodiments, the one or more agents are added to the culture medium at a first concentration during the first time period. In some embodiments, the one or more agents are added to the culture medium at a second concentration during the second time period. In some embodiments, the first concentration is lower than the second concentration. In some embodiments, the in vitro culture process comprises an air-liquid interface setup.

In one aspect, the present disclosure provides systems and methods for enhancing immune cells obtained from patient derived organoids (PDOs). In some embodiments, the immune cells comprise tumor-reactive immune cells, e.g., TILs. In the present disclosure, the pre-Rapid Expansion Protocol or pre-REP step involves treating the immune cells using different concentrations of cytokine, e.g., IL-2, during a first time period and a second time period. In some instances, the pre-REP step described herein discloses treating the immune cells during the first time period using a low concentration of cytokine before treating the immune cells with a higher concentration of cytokine during the second time period. Treating immune cells, e.g., TILs, with a low concentration of cytokine during the first time period in the pre-REP step helps maintain the immune cells in vitro and also prevents the immune cells from dying. In some instances, immune cells, e.g., TILs, obtained using the protocol described herein have a better tumor killing capacity and are more reactive to tumor cells when compared to immune cells, e.g., TILs, obtained from the standard protocol, which uses one concentration of the cytokine. Thus, the present disclosure provides systems and methods to generate and expand effective immune cells for cancer immunotherapy and other applications, such as immune cells that are more effective for cancer immunotherapy than the standard protocol. In some embodiments, the immune cells comprise tumor infiltrating lymphocytes (TILs). In some embodiments, the TILs comprise T cells. In some embodiments, the immune cells, e.g., TILs, comprises T cells. In some embodiments, the T cells comprise activated T cells, naïve CD8+ T cells, cytotoxic CD8+ T cells, naïve CD4+ T cells, helper T cells, e.g. T_{H}1, T_{H}2, T_{H}9, T_{H}17, T_{H}22, T_{FH}; memory T cells, e.g. central memory T cells, T stem cell memory cells (TSCM), effector memory T cells, NKT cells, or γd T cells.

In some embodiments, the one or more agents comprises a cytokine. In various embodiments, the cytokine used in the pre-REP protocol comprises IL-2, an IL-2 variant, IL-7, an IL-7 variant, IL-15, an IL-15 variant, IL-18, an IL-18 variant, IL-21, an IL-21 variant, or combination thereof. Variants of cytokine can comprise one or more mutations in the amino acid sequence compared to wild-type sequence. Variants of cytokine can be used to modulate a target signaling pathway. Examples of variants of cytokine include, but are not limited to, mutated variant or truncated variant.

In various embodiments, the concentration of cytokine added in the pre-REP step during the first time period is at least about 10 IU/mL, at least about 20 IU/mL, at least about 30 IU/mL, at least about 40 IU/mL, at least about 50 IU/mL, at least about 60 IU/mL, at least about 70 IU/mL, at least about 80 IU/mL, at least about 90 IU/mL, at least about 100 IU/mL, at least about 500 IU/mL, at least about 1000 IU/mL, at least about 2000 IU/mL, at least about 3000 IU/mL, at least about 4000 IU/mL, at least about 5000 IU/mL, at least about 6000 IU/mL, at least about 7000 IU/mL, at least about 8000 IU/mL, at least about 9000 IU/mL, or at least about 10000 IU/mL. In some embodiments, the concentration of cytokine added in the pre-REP step during the first time period is at most about 50 IU/mL, at most about 60 IU/mL, at most about 70 IU/mL, at most about 80 IU/mL, at most about 90 IU/mL, at most about 100 IU/mL, at most about 500 IU/mL, at most about 1000 IU/mL, at most about 2000 IU/mL, at most about 3000 IU/mL, at most about 4000 IU/mL, at most about 5000 IU/mL, at most about 6000 IU/mL, at most about 7000 IU/mL, at most about 8000 IU/mL, at most about 9000 IU/mL, at most about 10000 IU/mL, at most about 11000 IU/mL, or at most about 12000 IU/mL. In various embodiments, the concentration of cytokine added in the pre-REP step during the first time period is about 10 IU/mL, about 20 IU/mL, about 30 IU/mL, about 40 IU/mL, about 50 IU/mL, about 60 IU/mL, about 70 IU/mL, about 80 IU/mL, about 90 IU/mL, about 100 IU/mL, about 150 IU/mL, about 500 IU/mL, about 1000 IU/mL, about 2000 IU/mL, about 3000 IU/mL, about 4000 IU/mL, about 5000 IU/mL, about 6000 IU/mL, about 7000 IU/mL, about 8000 IU/mL, about 9000 IU/mL, or about 10000 IU/mL. In some embodiments, the concentration of cytokine added in the pre-REP step during the first time period is at least about 10 IU/mL. In some embodiments, the concentration of cytokine added in the pre-REP step during the first time period is about 50 IU/mL. In some embodiments, the concentration of cytokine added in the pre-REP step during the first time period is about 150 IU/mL.

In some embodiments, the concentration of cytokine added in the pre-REP step during the first time period is from about 10 IU/mL to about 12000 IU/mL, from about 20 IU/mL to about 12000 IU/mL, from about 30 IU/mL to about 12000 IU/mL, from about 40 IU/mL to about 12000 IU/mL, from about 50 IU/mL to about 12000 IU/mL, from about 60 IU/mL to about 12000 IU/mL, from about 70 IU/mL to about 12000 IU/mL, from about 80 IU/mL to about 12000 IU/mL, from about 90 IU/mL to about 12000 IU/mL, from about 100 IU/mL to about 12000 IU/mL, from about 500 IU/mL to about 12000 IU/mL, from about 1000 IU/mL to about 12000 IU/mL, from about 2000 IU/mL to about 12000 IU/mL, from about 3000 IU/mL to about 12000 IU/mL, from about 4000 IU/mL to about 12000 IU/mL, from about 5000 IU/mL to about 12000 IU/mL, from about 6000 IU/mL to about 12000 IU/mL, from about 7000 IU/mL to about 12000 IU/mL, from about 8000 IU/mL to about 12000 IU/mL, from about 9000 IU/mL to 12000 about IU/mL, or from about 10000 IU/mL to about 12000 IU/mL.

In some embodiments, the concentration of the cytokine added during the first time period in the pre-REP step is about 10 IU/mL to about 10000 IU/mL, about 20 IU/mL to about 10000 IU/mL, about 30 IU/mL to about 10000 IU/mL, about 40 IU/mL to about 10000 IU/mL, about 50 IU/mL to about 10000 IU/mL, about 60 IU/mL to about 10000 IU/mL, about 70 IU/mL to about 10000 IU/mL, about 80 IU/mL to about 10000 IU/mL, about 90 IU/mL to about 10000 IU/mL, about 100 IU/mL to about 10000 IU/mL, about 500 IU/mL to about 10000 IU/mL, about 1000 IU/mL to about 10000 IU/mL, about 2000 IU/mL to about 10000 IU/mL, about 3000 IU/mL to about 10000 IU/mL, about 4000 IU/mL to about 10000 IU/mL, about 5000 IU/mL to about 10000 IU/mL, about 6000 IU/mL to about 10000 IU/mL, about 7000 IU/mL to about 10000 IU/mL, about 8000 IU/mL to about 10000 IU/mL, or about 9000 IU/mL to about 10000 IU/mL. In some embodiments, the concentration of cytokine added in the pre-REP step during the first time period is about 50 IU/mL to about 10000 IU/mL.

In various embodiments, the first time period of the pre-REP step is at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, or at least about 15 days. In some embodiments, the first time period of the pre-REP step is at most about 1 day, at most about 2 days, at most about 3 days, at most about 4 days, at most about 5 days, at most about 6 days, at most about 7 days, at most about 8 days, at most about 9 days, at most about 10 days, at most about 11 days, at most about 12 days, at most about 13 days, at most about 14 days, or at most about 15 days. In some embodiments, the first time period of the pre-REP step is from about 1 to about 14 days, about 2 to about 14 days, about 3 to about 14 days, about 4 to about 14 days, about 5 to about 14 days, about 6 to about 14 days, about 7 to about 14 days, about 8 to about 14 days, or about 9 to about 14 days. In some embodiments, the first time period of the pre-REP is about 7 days to about 14 days. In various embodiments, the first time period of the pre-REP step is about 1 day, about 2 days, about 3 days, about 4 days, about 5 days, about 6 days, about 7 days, about 8 days, about 9 days, about 10 days, about 11 days, about 12 days, about 13 days, about 14 days, or about 15 days. In some embodiments, the first time period of the pre-REP step is at least about 1 day. In some embodiments, the first time period of the pre-REP step is about 7 days. In some embodiments, the first time period of the pre-REP step is about 7 to about 14 days.

In various embodiments, the concentration of cytokine added in the pre-REP step during the second time period is at least about 10 IU/mL, at least about 20 IU/mL, at least about 30 IU/mL, at least about 40 IU/mL, at least about 50 IU/mL, at least about 60 IU/mL, at least about 70 IU/mL, at least about 80 IU/mL, at least about 90 IU/mL, at least about 100 IU/mL, at least about 500 IU/mL, at least about 1000 IU/mL, at least about 2000 IU/mL, at least about 3000 IU/mL, at least about 4000 IU/mL, at least about 5000 IU/mL, at least about 6000 IU/mL, at least about 7000 IU/mL, at least about 8000 IU/mL, at least about 9000 IU/mL, or at least about 10000 IU/mL. In some embodiments, the concentration of cytokine used in the pre-REP step during the second time period is at most about 50 IU/mL, at most about 60 IU/mL, at most about 70 IU/mL, at most about 80 IU/mL, at most about 90 IU/mL, at most about 100 IU/mL, at most about 500 IU/mL, at most about 1000 IU/mL, at most about 2000 IU/mL, at most about 3000 IU/mL, at most about 4000 IU/mL, at most about 5000 IU/mL, at most about 6000 IU/mL, at most about 7000 IU/mL, at most about 8000 IU/mL, at most about 9000 IU/mL, at most about 10000 IU/mL, at most about 11000 IU/mL, or at most about 12000 IU/mL. In various embodiments, the concentration of cytokine added in the pre-REP step during the second time period is about 10 IU/mL, about 20 IU/mL, about 30 IU/mL, about 40 IU/mL, about 50 IU/mL, about 60 IU/mL, about 70 IU/mL, about 80 IU/mL, about 90 IU/mL, about 100 IU/mL, about 500 IU/mL, about 1000 IU/mL, about 2000 IU/mL, about 3000 IU/mL, about 4000 IU/mL, about 5000 IU/mL, about 6000 IU/mL, about 7000 IU/mL, about 8000 IU/mL, about 9000 IU/mL, or about 10000 IU/mL. In some embodiments, the concentration of cytokine added in the pre-REP step during the second time period is at least about 4000 IU/mL. In some embodiments, the concentration of cytokine added in the pre-REP step during the second time period is about 6000 IU/mL.

In some embodiments, the concentration of cytokine added in the pre-REP step during the second time period is from about 10 IU/mL to about 12000 IU/mL, from about 20 IU/mL to about 12000 IU/mL, from about 30 IU/mL to about 12000 IU/mL, from about 40 IU/mL to about 12000 IU/mL, from about 50 IU/mL to about 12000 IU/mL, from about 60 IU/mL to about 12000 IU/mL, from about 70 IU/mL to about 12000 IU/mL, from about 80 IU/mL to about 12000 IU/mL, from about 90 IU/mL to about 12000 IU/mL, from about 100 IU/mL to about 12000 IU/mL, from about 500 IU/mL to about 12000 IU/mL, from about 1000 IU/mL to about 12000 IU/mL, from about 2000 IU/mL to about 12000 IU/mL, from about 3000 IU/mL to about 12000 IU/mL, from about 4000 IU/mL to about 12000 IU/mL, from about 5000 IU/mL to about 12000 IU/mL, from about 6000 IU/mL to about 12000 IU/mL, from about 7000 IU/mL to about 12000 IU/mL, from about 8000 IU/mL to about 12000 IU/mL, from about 9000 IU/mL to about 12000 IU/mL, or from about 10000 IU/mL to about 12000 IU/mL.

In some embodiments, the concentration of the cytokine added during the second time period in the pre-REP step is from about 10 IU/mL to about 10000 IU/mL, from about 20 IU/mL to about 10000 IU/mL, from about 30 IU/mL to about 10000 IU/mL, from about 40 IU/mL to about 10000 IU/mL, from about 50 IU/mL to about 10000 IU/mL, from about 60 IU/mL to about 10000 IU/mL, from about 70 IU/mL to about 10000 IU/mL, from about 80 IU/mL to about 10000 IU/mL, from about 90 IU/mL to about 10000 IU/mL, from about 100 IU/mL to about 10000 IU/mL, from about 500 IU/mL to about 10000 IU/mL, from about 1000 IU/mL to about 10000 IU/mL, from about 2000 IU/mL to about 10000 IU/mL, from about 3000 IU/mL to about 10000 IU/mL, from about 4000 IU/mL to about 10000 IU/mL, from about 5000 IU/mL to about 10000 IU/mL, from about 6000 IU/mL to about 10000 IU/mL, from about 7000 IU/mL to about 10000 IU/mL, from about 8000 IU/mL to about 10000 IU/mL, or from about 9000 IU/mL to about 10000 IU/mL. In some embodiments, the concentration of the cytokine added during the second time period in the pre-REP step is from about 5000 IU/mL to about 10000 IU/mL.

In various embodiments, the second time period of the pre-REP step is at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, or at least about 24 days. In some embodiments, the second time period of the pre-REP step is at most about 1 day, at most about 2 days, at most about 3 days, at most about 4 days, at most about 5 days, at most about 6 days, at most about 7 days, at most about 8 days, at most about 9 days, at most about 10 days, at most about 11 days, at most about 12 days, at most about 13 days, at most about 14 days, at most about 15 days, at most about 16 days, at most about 17 days, at most about 18 days, at most about 19 days, at most about 20 days, at most about 21 days, at most about 22 days, at most about 23 days, or at most about 24 days. In some embodiments, the second time period of the pre-REP step is from about 1 to about 24 days, from about 2 to about 24 days, from about 3 to about 24 days, from about 4 to about 24 days, from about 5 to about 24 days, from about 6 to about 24 days, from about 7 to about 24 days, from about 8 to about 24 days, from about 9 to about 24 days, from about 10 to about 24 days, from about 11 to about 24 days, from about 12 to about 24 days, from about 13 to about 24 days, from about 14 to about 24 days, from about 15 to about 24 days, from about 16 to about 24 days, from about 17 to about 24 days, from about 18 to about 24 days, from about 19 to about 24 days, from about 20 to about 24 days, from about 21 to about 24 days, from about 22 to about 24 days, or from about 23 to about 24 days. In some embodiments, the second time period of the pre-REP step is about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, or about 24 days. In some embodiments, the second time period of the pre-REP step is at least about 1 day. In some embodiments, the second time period of the pre-REP step is about 11 days. In some embodiments, the second time period of the pre-REP step is about 7 to 14 days. In some embodiments, the second time period of the pre-REP step is about 11 to 14 days.

In some embodiments, the method further comprises providing additional reagents during the first time period or the second time period, wherein the additional reagents comprise one or more of PD-1, CD39, 4-1BB-positive T cells, or CXCR3-binding chemokines. In some embodiments, the CXCR3-binding chemokines comprise CXCL9 or CXCL10. In some embodiments, the method further comprises providing additional reagents during the first time period or the second time period, wherein the additional reagents comprise interferons. In some embodiments, the method further comprises providing additional reagents during the first time period or the second time period, wherein the additional reagents comprise checkpoint inhibitors. In some embodiments, the method further comprises providing additional reagents during the first time period or the second time period, wherein the additional reagents comprise TLR3, TLR7, TLR9, or other TLR agonists. In some embodiments, the method further comprises providing additional reagents during the first time period or the second time period, wherein the additional reagents comprise modulators of RIG-I-like receptors, modulators of NOD-like receptors, modulators of C-type lectin receptors, modulators of STING, or combination thereof.

In some embodiments, the method further comprises combining tumor organoids with immune cells obtained from other sources. In some embodiments, the other sources comprise peripheral blood cells or organoids grown from lymphoid tissue. In some embodiments, the method further comprises stimulating antigen presentation. In some embodiments, the method further comprises depletion of immune inhibitory cell types. In some embodiments, the immune inhibitory cell types comprise Tregs, myeloid derived suppressor cells, TAMs, vascular endothelial cells, or CAFs.

In some embodiments, the method further comprises negative selection of bystander tumor reactive immune cells. In some embodiments, the method further comprises knock-down exhaustion regulator. In some embodiments, the knock-down exhaustion regulator is TOX.

In some embodiments, the method comprises providing one or more tumor antigens during the first time period or the second time period. In some embodiments, the providing of one or more tumor antigens comprises providing cells expressing the tumor antigens.

In some embodiments, after the pre-REP, the immune cells are collected for expansion using Rapid Expansion Protocol (REP). In some embodiments, after the pre-REP, the immune cells are collected for other applications. In some embodiments, after the pre-REP, the immune cells are cryopreserved. In some embodiments, the immune cells are cryorecovered after cryopreservation. In some embodiments, the method further comprises cryopreserving the immune cells after collecting the immune cells from the one or more tissue samples. In some embodiments, the immune cells comprise tumor infiltrating lymphocytes. In some embodiments, the tumor infiltrating lymphocytes comprise T cells.

### C. Rapid Expansion Protocol (REP)

After the immune cells, e.g., TILs, undergo activation as described in the pre-REP step, next the immune cells are subjected to expansion. During the REP, the immune cells, e.g., TILs, are rapidly expanded by exposing the TILs to a cytokine, an antibody, a modulator, feeder cells, or any combination thereof. Further, these immune cells, e.g., TILs, can be reprogrammed into stem cell-like properties in this REP step, such as via Notch signaling pathway activation and interferon gamma (IFNγ) inhibition.

In one aspect, the present disclosure provides a method of generating ALI derived immune cells, wherein the method further comprises (d) expanding the immune cells after collecting the immune cells from the one or more tissue samples using one or more agents for a time period. In some embodiments, the method further comprises (e) collecting the immune cells after expanding the immune cells obtained from the isolation of the immune cells from the one or more tissue samples. In some embodiments, the method further comprises, after collecting the immune cells after the expansion, (f) cryopreserving the immune cells. In some embodiments, the immune cells comprise tumor-reactive immune cells, e.g., TILs. In some embodiments, the immune cells comprise tumor infiltrating lymphocytes. In some embodiments, the tumor infiltrating lymphocytes comprise T cells. In some embodiments, the T cells comprise activated T cells, naïve CD8+ T cells, cytotoxic CD8+ T cells, naïve CD4+ T cells, helper T cells, e.g. TH1, TH2, TH9, TH17, TH22, TFH; memory T cells, e.g. central memory T cells, T stem cell memory cells (TSCM), effector memory T cells, NKT cells, or γd T cells.

In some embodiments, the immune cells, e.g., TILs, are rapidly expanded through the REP step. In some embodiments, the expanding of the immune cells does not comprise an air-liquid interface setup. In some embodiments, the immune cells, e.g., TILs, are expanded in the non air-liquid interface (non-ALI) culture system. In some embodiments, the immune cells, e.g., TILs, used in the REP step is collected from the pre-REP step as described above. In some embodiments, the REP step comprises culturing the immune cells, e.g., TILs, in a non-ALI culture. In some embodiments, the one or more agents used in the REP step comprise a cytokine, an antibody, a modulator, or any combination thereof. In some embodiments, the REP culture comprises cytokines, an antibody, a modulator, feeder cells, or any combination thereof. In some embodiments, the immune cells, e.g., TILs, comprises T cells.

In some embodiments, the cytokines used in the REP culture comprise IL-2, an IL-2 variant, IL-7, an IL-7 variant, IL-15, an IL-15 variant, IL-18, an IL-18 variant, IL-21, an IL-21 variant, or combination thereof. Variants of cytokine can comprise one or more mutations in the amino acid sequence compared to wild-type sequence. Variants of cytokine can be used to modulate a target signaling pathway. Examples of variants of cytokine include, but are not limited to, mutated variant or truncated variant.

In some embodiments, the cytokine is added in the REP step at a concentration of at least about 10 IU/mL, at least about 20 IU/mL, at least about 30 IU/mL, at least about 40 IU/mL, at least about 50 IU/mL, at least about 60 IU/mL, at least about 70 IU/mL, at least about 80 IU/mL, at least about 90 IU/mL, at least about 100 IU/mL, at least about 500 IU/mL, at least about 1000 IU/mL, at least about 2000 IU/mL, at least about 3000 IU/mL, at least about 4000 IU/mL, at least about 5000 IU/mL, at least about 6000 IU/mL, at least about 7000 IU/mL, at least about 8000 IU/mL, at least about 9000 IU/mL, or at least about 10000 IU/mL. In some embodiments, the cytokine is added at a concentration of at least about 2000 IU/mL. In some embodiments, the cytokine is added in the REP step at a concentration of at most about 50 IU/mL, at most about 60 IU/mL, at most about 70 IU/mL, at most about 80 IU/mL, at most about 90 IU/mL, at most about 100 IU/mL, at most about 500 IU/mL, at most about 1000 IU/mL, at most about 2000 IU/mL, at most about 3000 IU/mL, at most about 4000 IU/mL, at most about 5000 IU/mL, at most about 6000 IU/mL, at most about 7000 IU/mL, at most about 8000 IU/mL, at most about 9000 IU/mL, at most about 10000 IU/mL, at most about 11000 IU/mL, or at most about 12000 IU/mL. In various embodiments, the cytokine is added in the REP step at a concentration of about 10 IU/mL, about 20 IU/mL, about 30 IU/mL, about 40 IU/mL, about 50 IU/mL, about 60 IU/mL, about 70 IU/mL, about 80 IU/mL, about 90 IU/mL, about 100 IU/mL, about 500 IU/mL, about 1000 IU/mL, about 2000 IU/mL, about 3000 IU/mL, about 4000 IU/mL, about 5000 IU/mL, about 6000 IU/mL, about 7000 IU/mL, about 8000 IU/mL, about 9000 IU/mL, or about 10000 IU/mL. In some embodiments, the cytokine is added in the REP step at a concentration of at least about 1000 IU/mL. In some embodiments, the cytokine is added in the REP step at a concentration of about 3000 IU/mL.

In some embodiments, the cytokine is added in the REP step at a concentration of from about 10 IU/mL to about 12000 IU/mL, from about 20 IU/mL to about 12000 IU/mL, from about 30 IU/mL to about 12000 IU/mL, from about 40 IU/mL to about 12000 IU/mL, from about 50 IU/mL to about 12000 IU/mL, from about 60 IU/mL to about 12000 IU/mL, from about 70 IU/mL to about 12000 IU/mL, from about 80 IU/mL to about 12000 IU/mL, from about 90 IU/mL to about 12000 IU/mL, from about 100 IU/mL to about 12000 IU/mL, from about 500 IU/mL to about 12000 IU/mL, from about 1000 IU/mL to about 12000 IU/mL, from about 2000 IU/mL to about 12000 IU/mL, from about 3000 IU/mL to about 12000 IU/mL, from about 4000 IU/mL to about 12000 IU/mL, from about 5000 IU/mL to about 12000 IU/mL, from about 6000 IU/mL to about 12000 IU/mL, from about 7000 IU/mL to about 12000 IU/mL, from about 8000 IU/mL to about 12000 IU/mL, from about 9000 IU/mL to about 12000 IU/mL, or from about 10000 IU/mL to about 12000 IU/mL.

In some embodiments, the cytokine is added in the REP step at a concentration of from about 10 IU/mL to about 10000 IU/mL, from about 20 IU/mL to about 10000 IU/mL, from about 30 IU/mL to about 10000 IU/mL, from about 40 IU/mL to about 10000 IU/mL, from about 50 IU/mL to about 10000 IU/mL, from about 60 IU/mL to about 10000 IU/mL, from about 70 IU/mL to about 10000 IU/mL, from about 80 IU/mL to about 10000 IU/mL, from about 90 IU/mL to about 10000 IU/mL, from about 100 IU/mL to about 10000 IU/mL, from about 500 IU/mL to about 10000 IU/mL, from about 1000 IU/mL to about 10000 IU/mL, from about 2000 IU/mL to about 10000 IU/mL, from about 3000 IU/mL to about 10000 IU/mL, from about 4000 IU/mL to about 10000 IU/mL, from about 5000 IU/mL to about 10000 IU/mL, from about 6000 IU/mL to about 10000 IU/mL, from about 7000 IU/mL to about 10000 IU/mL, from about 8000 IU/mL to about 10000 IU/mL, or from about 9000 IU/mL to about 10000 IU/mL. In some embodiments, the cytokine added in the REP step at a concentration of from about 2000 IU/mL to about 10000 IU/mL.

In some embodiments, the time period of the REP step is at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, or at least about 24 days. In some embodiments, the time period of the REP step is at most about 1 day, at most about 2 days, at most about 3 days, at most about 4 days, at most about 5 days, at most about 6 days, at most about 7 days, at most about 8 days, at most about 9 days, at most about 10 days, at most about 11 days, at most about 12 days, at most about 13 days, at most about 14 days, at most about 15 days, at most about 16 days, at most about 17 days, at most about 18 days, at most about 19 days, at most about 20 days, at most about 21 days, at most about 22 days, at most about 23 days, or at most about 24 days. In some embodiments, the time period of the REP is from about 1 to about 24 days, from about 2 to about 24 days, from about 3 to about 24 days, from about 4 to about 24 days, from about 5 to about 24 days, from about 6 to about 24 days, from about 7 to about 24 days, from about 8 to about 24 days, from about 9 to about 24 days, from about 10 to about 24 days, from about 11 to about 24 days, from about 12 to about 24 days, from about 13 to about 24 days, from about 14 to about 24 days, from about 15 to about 24 days, from about 16 to about 24 days, from about 17 to about 24 days, from about 18 to about 24 days, from about 19 to about 24 days, from about 20 to about 24 days, from about 21 to about 24 days, from about 22 to about 24 days, or from about 23 to about 24 days. In some embodiments, the time period of the REP is about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, or about 24 days. In some embodiments, the time period of the REP is at least about 7 days. In some embodiments, the time period of the REP is about 14 days. In some embodiments, the time period of the REP is from about 7 to about 14 days.

In some embodiments, the antibody used in the REP culture comprises an anti-CD3 antibody. In some embodiments, the anti-CD3 antibody is a monoclonal antibody. In some embodiments, the anti-CD3 monoclonal antibody is obtained from Clone OKT3 or Clone UCHT1. In some embodiments, a concentration of the anti-CD3 antibody added is at least about 1 ng/mL, at least about 3 ng/mL, at least about 5 ng/mL, at least about 7 ng/mL, at least about 10 ng/mL, at least about 15, ng/mL, at least about 20 ng/mL, at least about 25 ng/mL, at least about 30 ng/mL, at least about 35 ng/mL, at least about 40 ng/mL, at least about 45 ng/mL, at least about 50 ng/mL, at least about 55 ng/mL, at least about 60 ng/mL, at least about 65 ng/mL, at least about 70 ng/mL, at least about 75 ng/mL, at least about 80 ng/mL, at least about 85 ng/mL, at least about 90 ng/mL, at least about 95 ng/mL, or at least about 100 ng/mL. In some embodiments, the concentration of the anti-CD3 antibody added is at most about 1 ng/mL, at most about 3 ng/mL, at most about 5 ng/mL, at most about 7 ng/mL, at most about 10 ng/mL, at most about 15, ng/mL, at most about 20 ng/mL, at most about 25 ng/mL, at most about 30 ng/mL, at most about 35 ng/mL, at most about 40 ng/mL, at most about 45 ng/mL, at most about 50 ng/mL, at most about 55 ng/mL, at most about 60 ng/mL, at most about 65 ng/mL, at most about 70 ng/mL, at most about 75 ng/mL, at most about 80 ng/mL, at most about 85 ng/mL, at most about 90 ng/mL, at most about 95 ng/mL, or at most about 100 ng/mL. In some embodiments, the concentration of the anti-CD3 antibody added is about 1 ng/mL, about 3 ng/mL, about 5 ng/mL, about 7 ng/mL, about 10 ng/mL, about 15, ng/mL, about 20 ng/mL, about 25 ng/mL, about 30 ng/mL, about 35 ng/mL, about 40 ng/mL, about 45 ng/mL, about 50 ng/mL, about 55 ng/mL, about 60 ng/mL, about 65 ng/mL, about 70 ng/mL, about 75 ng/mL, about 80 ng/mL, about 85 ng/mL, about 90 ng/mL, about 95 ng/mL, or about 100 ng/mL. In some embodiments, the concentration of the anti-CD3 antibody added is about 10 ng/mL. In some embodiments, a concentration of the anti-CD3 antibody added is about 30 ng/mL.

In some embodiments, the one or more agents in REP step further comprise irradiated feeder cells. In some embodiments, the irradiated feeder cells are irradiated allogeneic PBMC-derived feeder cells. In some embodiments, the ratio of the immune cells to the irradiated feeder cells is about 1:10, about 1:50, about 1:100, about 1:150, or about 1:200. In some embodiments, the ratio of the immune cells to the irradiated feeder cells is about 1:100.

In some embodiments, the feeder cells used in the REP culture comprise peripheral blood mononuclear cell (PBMC) feeders. In some embodiments, the feeder cells used in the REP culture are derived from PBMC feeders. In some embodiments, the PBMC feeders are irradiated feeder cells. In some embodiments, the PBMC feeders are allogeneic PBMC feeder cells. In some embodiments, the PBMC feeders are irradiated allogeneic PBMC feeder cells.

In some embodiments, the immune cells, e.g., TILs, are expanded in the REP step for up to about 5 days, up to about 6 days, up to about 7 days, up to about 8 days, up to about 9 days, up to about 10 days, up to about 11 days, up to about 12 days, up to about 13 days, up to about 14 days, up to about 15 days, up to about 16 days, up to about 17 days, up to about 18 days, up to about 19 days, up to about 20 days, up to about 21 days or greater. In some embodiments, the immune cells, e.g., TILs, are expanded in the REP step for at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, or greater. In some embodiments, the immune cells, e.g., TILs, are expanded in the REP step for about 14 days.

In some embodiments, once expanded, immune cells, e.g., TILs, can be assayed for functional activity. In some embodiments, once expanded, the immune cells, e.g., TILs, can be administered to a patient for immunotherapy. In some embodiments, once expanded, the immune cells, e.g., TILs, can be collected for other applications, e.g., in vitro modeling, drug screening, etc. In some embodiments, the immune cells, e.g., TILs, comprises T cells.

In some embodiments, the method further comprises combining tumor organoids with immune cells obtained from other sources. In some embodiments, the other sources comprise peripheral blood cells or organoids grown from lymphoid tissue. In some embodiments, the method further comprises stimulating antigen presentation. In some embodiments, the method further comprises depletion of immune inhibitory cell types. In some embodiments, the immune inhibitory cell types comprise Tregs, myeloid derived suppressor cells, TAMs, vascular endothelial cells, or CAFs.

In some embodiments, the method further comprises negative selection of bystander tumor infiltrating immune cells. In some embodiments, the method further comprises knock-down exhaustion regulator. In some embodiments, the knock-down exhaustion regulator is TOX.

In some embodiments, the method further comprises reprogramming T cells. In some embodiments, during the REP step, the immune cells, e.g., TILs, can be reprogrammed to have stem cell-like properties by inhibiting interferon-gamma (IFNγ) signaling pathway, activating Notch signaling pathway, or both. In some embodiments, the T cells are reprogrammed by activation of Notch signaling pathway during the REP step. In some embodiments, the T cells are reprogrammed by inhibiting IFNγ signaling. In some embodiments, the T cells are reprogrammed by both activating Notch signaling pathway and inhibiting IFNγ signaling pathway using the modulator. In some embodiments, the modulator can be an IFNγ modulator, a Notch modulator, or combination thereof. In some embodiments, the IFNγ modulator can inhibit IFNγ signaling pathways. In some embodiments, the IFNγ modulator comprises an IFNγ inhibitor. In some embodiments, the Notch modulator can activate Notch signaling pathways. In some embodiments, the Notch modulator comprises a Notch activator.

In some embodiments, the method further comprises identifying T cell receptors (TCR) to identify TCRs enriched in the air-liquid interface culture. In some embodiments, the identifying TCRs is performed using sequencing technology. In some embodiments, the method comprises providing one or more tumor antigens during the first time period or the second time period. In some embodiments, providing the tumor antigens comprises providing cells expressing the tumor antigens.

In some embodiments, after the REP step, the immune cells, e.g., TILs, can be cryopreserved. In some embodiments, the immune cells, e.g., TILs, can be cryorecovered after cryopreservation.

D. Reprogramming Air-Liquid Interface Tumor Infiltrating Lymphocytes (rALI TILs)

In some embodiments, methods and systems described herein can comprise reprogramming isolated, expanded, or cryopreserved TILs (e.g., ALI TILs) into a stem-like state. In some embodiments, the reprograming occurs during the REP step. In some embodiments, the reprogramming occurs before the REP step. In some embodiments, the reprogramming occurs after the REP step. In some embodiments, the isolated or cryopreserved TILs, e.g., T cells, comprise (i) central memory T cells (TCM), which can be characterized by CD45RA-CCR7+CD62L+ expression, (ii) tissue resident memory T cells (TRM), which can be identified by CD69+CD103+ expression, (iii) effector memory T cells (TEM), which can be identified by CD45RA-CCR7-CD62L-expression, (iv) stem cell memory T cells (TSCM), which can be characterized by CD45RA+CCR7+CD62L+CD95+ expression, (v) Naïve T cells, which can be characterized by CD45RA+CCR7+CD62L+ expression, and (vi) terminally differentiated effector memory cells (TEMRA), which can be identified by CD45RO-/CCR7—expression. In some embodiments, methods and systems described herein can reprogram one or more isolated TILs, e.g., T cells, into cells having a stem-like property (e.g., self-renewal capacity), such as memory T cells (TSCMs) or central memory T cells (TCMs). In some embodiments, the reprogrammed immune cells, e.g., T cells, can possess an improved lifespan, an increased self-renewal capacity, and/or an ability to differentiate into other cell lineage. In some embodiments, the methods and systems described herein can reprogram one or more terminally differentiated immune cells, e.g., T cells (e.g., effector memory T cells), into stem-like T cells, such as memory T cells (TSCMs). In some embodiments, the reprogrammed immune cells, e.g., T cells, can have enhanced persistence, proliferation, and/or efficacy in adoptive cellular therapy for different cancer types. In some embodiments, the reprogrammed immune cells, e.g., T cells, can be used in adoptive cellular therapy to treat cancer. In some embodiments, the cancer comprises glioblastoma. In some embodiments, the reprogrammed immune cells, e.g., T cells, can be used in adoptive cellular therapy to treat glioblastoma.

In some embodiments, the method further comprises reprograming the immune cells, e.g., T cells. In some embodiments, the immune cells, e.g., T cells, are reprogrammed by activation of Notch signaling pathway during the REP step, inhibition of interferon gamma (IFNγ) signaling during the REP step, or both. In some embodiments, methods and systems described herein can comprise treating isolated or cryopreserved TILs (e.g., ALI TILs) with one or more agents that facilitate the reprogramming of the immune cells, e.g., T, cells into TSCMs. In some embodiments, the one or more agents comprise modulators. In some embodiments, the modulators can enhance a signaling pathway that is known to convert activated T cells into stem memory T cells (TSCMs) (e.g., Notch signaling pathway). In some embodiments, the modulators can inhibit a signaling pathway that is known to inhibit the maintenance and diversity of stem-like immune cells, e.g., T cells, or to induce the terminal differentiation of memory immune cells, e.g., memory T cells, (e.g., IFNγ signaling pathway). In some embodiments, the modulators comprise a Notch signaling pathway modulator, an interferon gamma (IFNγ) modulator, or a combination thereof.

In some embodiments, the one or more agents comprise an IFNγ modulator. In some embodiments, the IFNγ modulator can inhibit IFNγ signaling pathways. In some embodiments, the IFNγ modulator comprises an IFNγ inhibitor. In some embodiments, the IFNγ inhibitor comprises an antibody, a small molecule, or a combination thereof. In some embodiments, the IFNγ inhibitor comprises IFNγ neutralizing antibodies or fragments thereof. In some embodiments, the IFNγ neutralizing antibodies can bind to the receptor. In some embodiments, the IFNγ neutralizing antibody can bind to circulating IFNγ. In some embodiments, the one or more agents comprise a protein tyrosine phosphatase that dephosphorylates the IFNγ receptor. In some embodiments, the one or more agents that block IFNγ signaling pathways comprise an inhibitor that targets proteins or signaling molecules downstream of IFNγ signaling pathway. In some embodiments, the one or more agents comprise an inhibitor of JAK/STAT signaling pathways. In some embodiments, the one or more agents comprises a small molecule inhibitor. In some embodiments, the treatment of immune cells, e.g., TILs, with the one or more agents described herein can result in expression changes of IFNγ induced genes, such as Gbp5, Irf1, and Ccl2 within 8 hours, 24 hours, 48 hours, or longer, after the treatment.

In some embodiments, the one or more agents comprise the Notch signaling pathway modulator. In some embodiments, the Notch signaling pathway modulator can activate Notch signaling pathways. In some embodiments, the Notch signaling pathway modulator comprises a Notch activator. In some embodiments, the Notch activator comprises an antibody, a small molecule, or a combination thereof. In some embodiments, the Notch activator comprises agonistic antibodies that target and activate the Notch receptor. In some embodiments, the one or more agents can be Delta-like 1 ligand. In some embodiments, the one or more agents can enhance expression of the downstream molecules of the Notch signaling pathway. In some embodiments, the one or more agents can be a small molecule agonist. In some embodiments, the one or more agents can be an agonistic monoclonal antibody (mAb). In some embodiments, the treatment of immune cells, e.g., TILs, with the one or more agents described herein can result in expression changes of Notch induced genes, such as Myc, Deltex1, and Hes1 within 8 hours, 24 hours, 48 hours, or longer, after the treatment.

In some embodiments, methods and systems described herein can comprise treating immune cells, e.g., TILs, with one or more agents to inhibit IFNγ signaling and activate Notch signaling. In some embodiments, the one or more agents comprise one or more modulators. In some embodiments, the one or more modulators comprise a first modulator that inhibits IFNγ signaling and a second modulator that activates Notch signaling. In some embodiments, the one or more modulators comprise Notch signaling pathway modulator, interferon gamma (IFNγ) modulator, or combination thereof. In some embodiments, the one or more modulators are added separately. In some embodiments, the one or more modulators are added sequentially. In some embodiments, the one or more modulators are added simultaneously. In some embodiments, the one or more agents described herein can be a mAb cocktail that simultaneously targets IFNγ inhibition and Notch activation. In some embodiments, the mAb cocktail can be applied throughout or for the last about 1 day, throughout or for the last about 2 days, throughout or for the last about 3 days, throughout or for the last about 4 days, throughout or for the last about 5 days, throughout or for the last about 6 days, throughout or for the last about 7 days, throughout or for the last about 8 days, throughout or for the last about 9 days, throughout or for the last about 10 days, throughout or for the last about 11 days, throughout or for the last about 12 days, throughout or for the last about 13 days, or throughout or for the last about 14 days of the REP step during culture. In some embodiments, the method may further comprise treating TIL culture during the REP step with one or more cytokines. In some embodiments, the cytokines can be IL-2, an IL-2 variant, IL-7, an IL-7 variant, IL-15, an IL-15 variant, IL-18, an IL-18 variant, IL-21, an IL-21 variant, or combination thereof. Variants of cytokine can comprise one or more mutations in the amino acid sequence compared to wild-type sequence. Variants of cytokine can be used to modulate a target signaling pathway. Examples of variants of cytokine include, but are not limited to, mutated variant or truncated variant.

The method described herein can yield a TSCM population of about 2% to about 50% of the TIL population after the reprogramming. In some cases, the method described herein can yield a TSCM population of about 2% to about 4%, about 2% to about 6%, about 2% to about 8%, about 2% to about 10%, about 2% to about 15%, about 2% to about 20%, about 2% to about 25%, about 2% to about 30%, about 2% to about 35%, about 2% to about 40%, about 2% to about 50%, about 4% to about 6%, about 4% to about 8%, about 4% to about 10%, about 4% to about 15%, about 4% to about 20%, about 4% to about 25%, about 4% to about 30%, about 4% to about 35%, about 4% to about 40%, about 4% to about 50%, about 6% to about 8%, about 6% to about 10%, about 6% to about 15%, about 6% to about 20%, about 6% to about 25%, about 6% to about 30%, about 6% to about 35%, about 6% to about 40%, about 6% to about 50%, about 8% to about 10%, about 8% to about 15%, about 8% to about 20%, about 8% to about 25%, about 8% to about 30%, about 8% to about 35%, about 8% to about 40%, about 8% to about 50%, about 10% to about 15%, about 10% to about 20%, about 10% to about 25%, about 10% to about 30%, about 10% to about 35%, about 10% to about 40%, about 10% to about 50%, about 15% to about 20%, about 15% to about 25%, about 15% to about 30%, about 15% to about 35%, about 15% to about 40%, about 15% to about 50%, about 20% to about 25%, about 20% to about 30%, about 20% to about 35%, about 20% to about 40%, about 20% to about 50%, about 25% to about 30%, about 25% to about 35%, about 25% to about 40%, about 25% to about 50%, about 30% to about 35%, about 30% to about 40%, about 30% to about 50%, about 35% to about 40%, about 35% to about 50%, or about 40% to about 50% of the TIL population, after the reprogramming. The method described herein can yield a TSCM population of about 2%, about 4%, about 6%, about 8%, about 10%, about 15%, about 20%, about 25%, about 30%, about 35%, about 40%, or about 50% of the TIL population, after the reprogramming. The methods described herein can yield a TSCM population of at least about 2%, at least about 4%, at least about 6%, at least about 8%, at least about 10%, at least about 15 00 at least about 20%, at least about 25%, at least about 30%, at least about 35%, or at least about 40% of the TIL population, after the reprograming. The methods described herein can yield a TSCM population of at most about 4%, at most about 6%, at most about 8%, at most about 10%, at most about 15%, at most about 20%, at most about 25%, at most about 30%, at most about 35%, at most about 40%, or at most about 50% of the TIL population, after the reprogramming.

The methods described herein can yield a TCM population of about 2% to about 50% of the TIL population, after the reprogramming. In some cases, the methods described herein can yield a TCM population of about 2% to about 4%, about 2% to about 6%, about 2% to about 8%, about 2% to about 10%, about 2% to about 15%, about 2% to about 20%, about 2% to about 25%, about 2% to about 30%, about 2% to about 35%, about 2% to about 40%, about 2% to about 50%, about 4% to about 6%, about 4% to about 8%, about 4% to about 10%, about 4% to about 15%, about 4% to about 20%, about 4% to about 25%, about 4% to about 30%, about 4% to about 35%, about 4% to about 40%, about 4% to about 50%, about 6% to about 8%, about 6% to about 10%, about 6% to about 15%, about 6% to about 20%, about 6% to about 25%, about 6% to about 30%, about 6% to about 35%, about 6% to about 40%, about 6% to about 50%, about 8% to about 10%, about 8% to about 15%, about 8% to about 20%, about 8% to about 25%, about 8% to about 30%, about 8% to about 35%, about 8% to about 40%, about 8% to about 50%, about 10% to about 15%, about 10% to about 20%, about 10% to about 25%, about 10% to about 30%, about 10% to about 35%, about 10% to about 40%, about 10% to about 50%, about 15% to about 20%, about 15% to about 25%, about 15% to about 30%, about 15% to about 35%, about 15% to about 40%, about 15% to about 50%, about 20% to about 25%, about 20% to about 30%, about 20% to about 35%, about 20% to about 40%, about 20% to about 50%, about 25% to about 30%, about 25% to about 35%, about 25% to about 40%, about 25% to about 50%, about 30% to about 35%, about 30% to about 40%, about 30% to about 50%, about 35% to about 40%, about 35% to about 50%, or about 40% to about 50% of the TIL population, after the reprogramming. The method described herein can yield a TCM population of about 2 00 about 4%, about 6%, about 8%, about 10%, about 15%, about 20%, about 25%, about 30 00 about 35%, about 40%, or about 50% of the TIL population, after the reprogramming. The method described herein can yield a TCM population of at least about 2%, at least about 4%, at least about 6%, at least about 8%, at least about 10%, at least about 15%, at least about 20%, at least about 25%, at least about 30%, at least about 35%, or at least about 40% of the TIL population, after the reprograming. The methods described herein can yield a TCM population of at most about 4%, at most about 6%, at most about 8%, at most about 10%, at most about 15%, at most about 20%, at most about 25%, at most about 30%, at most about 35%, at most about 40%, or at most about 50% of the TIL population, after the reprogramming.

In some embodiments, the method further comprises identifying T cell receptors (TCRs) to identify TCRs enriched in the air-liquid interface culture. In some embodiments, the identifying TCRs is performed using sequencing technology.

In some embodiments, after the REP step and reprogramming, the immune cells, e.g., rALI TILs, can be cryopreserved. In some embodiments, the immune cells, e.g., rALI TILs, are cryorecovered after cryopreservation.

### Characteristics of Air-Liquid Interface Immune Cells

In one aspect, the immune cells generated from the systems and methods described herein demonstrate a better tumor killing capacity compared to standard immune cells. In some embodiments, the immune cells generated from the systems and methods described herein are more reactive to tumor cells compared to standard immune cells (e.g., those immune cells generated from a standard protocol, as described herein) In some embodiments, the immune cells comprise lymphocytes, such as B cells and T cells; natural killer cells; dendritic cells; myeloid cells, such as monocytes, macrophages, eosinophils, mast cells, basophils, and granulocytes. In some instances, the immune cells comprise tumor infiltrating lymphocytes. In some embodiments, the tumor infiltrating lymphocytes comprise T cells. In some embodiments, the T cells comprise naïve CD8+ T cells, cytotoxic CD8+ T cells, naïve CD4+ T cells, helper T cells, e.g. TH1, TH2, T_{H}9, T_{H}17, T_{H}22, T_{FH}; memory T cells, e.g. central memory T cells, T stem cell memory cells (T_{SCM}), effector memory T cells, NKT cells, γδ T cells. In some embodiments, naïve T cells or terminally differentiated T cells can be reprogrammed to stem-like T cells (e.g., T stem cell memory cells).

In another aspect, the present disclosure provides a composition for treating a tumor comprising in vitro isolated immune cells, wherein the immune cells comprise tumor infiltrating lymphocytes expressing IFNγ, CD107a, or HLA-DR, wherein the tumor infiltrating lymphocyte population express higher IFNγ compared to tumor infiltrating lymphocytes obtained from standard protocol. In some instances, the immune cells comprise tumor infiltrating lymphocytes, TILs, or reprogrammed lymphocytes. In some instances, the population of the ALI TILs or reprogrammed ALI TILs (rALI TILs) obtained from the systems and methods described herein express higher IFNγ, CD107a, and/or HLA-DR compared to population of the STD TILs in response to specific stimulus. In some instances, the ALI TILs or rALI TILs express higher IFNγ and/or CD107a compared to the STD TILs.

In some instances, at least about 1%, at least about 3%, at least about 5%, at least about 10%, at least about 15%, at least about 20%, at least about 23%, at least about 30%, at least about 35%, at least about 40%, at least about 45%, at least about 50%, at least about 55%, or at least about 60% of ALI TIL or rALI TIL population obtained from methods and system described herein are IFNγ expressing cells. In some instances, at least about 5% of ALI TIL or rALI TIL population obtained from methods and system described herein are IFNγ expressing cells. In some instances, up to about 10%, up to about 15%, up to about 20%, up to about 23%, up to about 30%, up to about 35%, up to about 40%, up to about 45%, up to about 50%, up to about 55%, or up to about 60% of ALI TIL or rALI TIL population obtained from methods and system described herein are IFNγ expressing cells. In some instances, up to about 20% of ALI TIL or rALI TIL population obtained from methods and system described herein are IFNγ expressing cells. In some instances, about 5% to about 60%, about 10% to about 60%, about 15% to about 60%, about 20% to about 60%, about 25% to about 60%, about 30% to about 60%, 35% to about 60%, about 40% to about 60%, about 45% to about 60%, about 50% to about 60%, or about 55% to about 60% of ALI TIL or rALI TIL population obtained from methods and system described herein are IFNγ expressing cells. In some instances, about 5% to about 20%, about 10% to about 20%, or about 15% to about 20% of ALI TIL or rALI TIL population obtained from methods and system described herein are IFNγ expressing cells. In some instances, about 5% to about 20% of ALI TIL or rALI TIL population obtained from methods and system described herein are IFNγ expressing cells.

In some instances, at least about 1%, at least about 3%, at least about 5%, at least about 10%, at least about 15%, at least about 20%, at least about 23%, at least about 30%, at least about 35%, at least about 40%, at least about 45%, at least about 50%, at least about 55%, or at least about 60% of ALI TIL or rALI TIL population obtained from methods and system described herein are CD107a expressing cells. In some instances, at least about 3% of ALI TIL or rALI TIL population obtained from methods and system described herein are CD107a expressing cells. In some instances, up to about 10%, up to about 15%, up to about 20%, up to about 23%, up to about 30%, up to about 35%, up to about 40%, up to about 45%, up to about 50%, up to about 55%, or up to about 60% of ALI TIL or rALI TIL population obtained from methods and system described herein are CD107a expressing cells. In some instances, up to about 20% of ALI TIL or rALI TIL population obtained from methods and system described herein are CD107a expressing cells. In some instances, about 3% to about 60%, about 5% to about 60%, about 10% to about 60%, about 15% to about 60%, about 20% to about 60%, about 25% to about 60%, about 30% to about 60%, 35% to about 60%, about 40% to about 60%, about 45% to about 60%, about 50% to about 60%, or about 55% to about 60% of ALI TIL or rALI TIL population obtained from methods and system described herein are CD107a expressing cells. In some instances, about 3% to about 20%, 5% to about 20%, about 10% to about 20%, or about 15% to about 20% of ALI TIL or rALI TIL population obtained from methods and system described herein are CD107a expressing cells. In some instances, about 3% to about 20% of ALI TIL or rALI TIL population obtained from methods and system described herein are CD107a expressing cells.

In some instances, at least about 1%, at least about 3%, at least about 5%, at least about 10%, at least about 15%, at least about 20%, at least about 23%, at least about 30%, at least about 35%, at least about 40%, at least about 45%, at least about 50%, at least about 55%, at least about 60%, at least about 65%, or at least about 70% of ALI TIL or rALI TIL population obtained from methods and system described herein are HLA-DR expressing cells. In some instances, at least about 10% of ALI TIL or rALI TIL population obtained from methods and system described herein are HLA-DR expressing cells. In some instances, up to about 10%, up to about 15%, up to about 20%, up to about 23%, up to about 30%, up to about 35%, up to about 40%, up to about 45%, up to about 50%, up to about 55%, up to about 60%, up to about 65%, or up to about 70% of ALI TIL or rALI TIL population obtained from methods and system described herein are HLA-DR expressing cells. In some instances, up to about 20% of ALI TIL or rALI TIL population obtained from methods and system described herein are HLA-DR expressing cells. In some instances, about 5% to about 60%, about 10% to about 60%, about 15% to about 60%, about 20% to about 60%, about 25% to about 60%, about 30% to about 60%, 35% to about 60%, about 40% to about 60%, about 45% to about 60%, about 50% to about 60%, or about 55% to about 60% of ALI TIL or rALI TIL population obtained from methods and system described herein are HLA-DR expressing cells.

In some instances, expression markers of IFNγ, CD107a, and/or HLA-DR can be characterized at least at about 5 hours, at least at about 6 hours, at least at about 7 hours, at least at about 8 hours, at least at about 9 hours, at least at about 10 hours, at least at about 11 hours, at least at about 12 hours, at least at about 13 hours, at least at about 14 hours, at least at about 15 hours, at least at about 16 hours, at least at about 17 hours, at least at about 18 hours, at least at about 19 hours, at least at about 20 hours, at least at about 21 hours, at least at about 22 hours, at least at about 23 hours, at least at about 24 hours, at least at about 25 hours, at least at about 26 hours, at least at about 27 hours, at least at about 28 hours, at least at about 29 hours, at least at about 30 hours, at least at about 31 hours, at least at about 32 hours, at least at about 33 hours, at least at about 34 hours, at least at about 35 hours, at least at about 36 hours, at least at about 37 hours, at least at about 38 hours, at least at about 39 hours, at least at about 40 hours, at least at about 41 hours, at least at about 42 hours, at least at about 43 hours, at least at about 44 hours, at least at about 45 hours, at least at about 46 hours, at least at about 47 hours, at least at about 48 hours, at least at about 49 hours, at least at about 50 hours, at least at about 51 hours, at least at about 52 hours, at least at about 53 hours, at least at about 54 hours, at least at about 55 hours, at least at about 56 hours, at least at about 57 hours, at least at about 58 hours, at least at about 59 hours, at least at about 60 hours, or more than about 60 hours, after co-culturing with tumor cells. In some instances, the expression markers can be characterized using immunohistochemistry or Flow Cytometry.

In some instances, the ALI TILs or rALI TILs express higher level of Human Leukocyte Antigen—DR isotype (HLA-DR) compared to the STD TILs. In some instances, expression markers of HLA-DR can be characterized at least at about 5 hours, at least at about 6 hours, at least at about 7 hours, at least at about 8 hours, at least at about 9 hours, at least at about 10 hours, at least at about 11 hours, at least at about 12 hours, at least at about 13 hours, at least at about 14 hours, at least at about 15 hours, at least at about 16 hours, at least at about 17 hours, at least at about 18 hours, at least at about 19 hours, at least at about 20 hours, at least at about 21 hours, at least at about 22 hours, at least at about 23 hours, at least at about 24 hours, at least at about 25 hours, at least at about 26 hours, at least at about 27 hours, at least at about 28 hours, at least at about 29 hours, at least at about 30 hours, at least at about 31 hours, at least at about 32 hours, at least at about 33 hours, at least at about 34 hours, at least at about 35 hours, at least at about 36 hours, at least at about 37 hours, at least at about 38 hours, at least at about 39 hours, at least at about 40 hours, at least at about 41 hours, at least at about 42 hours, at least at about 43 hours, at least at about 44 hours, at least at about 45 hours, at least at about 46 hours, at least at about 47 hours, at least at about 48 hours, at least at about 49 hours, at least at about 50 hours, at least at about 51 hours, at least at about 52 hours, at least at about 53 hours, at least at about 54 hours, at least at about 55 hours, at least at about 56 hours, at least at about 57 hours, at least at about 58 hours, at least at about 59 hours, at least at about 60 hours, or more than 60 hours, after co-culturing with tumor cells. In some instances, the expression markers can be characterized using immunohistochemistry or Flow Cytometry.

In some instances, population of the ALI TILs or rALI TILs obtained from systems and methods described herein express higher HLA-DR compared to population of the STD TILs. In some instances, the HLA-DR expression level of ALI TILs or rALI TILs population obtained from methods and systems described herein is at least about 10%, at least about 15%, at least about 20%, at least about 23%, at least about 30%, at least about 35%, at least about 40%, at least about 45%, at least about 50%, at least about 55%, or at least about 60% higher compared to the STD TILs. In some instances, at least about 5%, at least about 10%, at least about 15%, at least about 20%, at least about 25%, at least about 30%, at least about 35%, at least about 40%, at least about 45%, at least about 50%, at least about 55%, at least about 60%, or at least about 65% of ALI TIL or rALI TIL population obtained from methods and system described herein are HLA-DR expressing cells.

In some instances, standard (“STD”) refers to immune cells obtained from a method involving obtaining and culturing immune cells, e.g., TILs, from fresh tumor tissue and treated with one concentration of cytokine during the pre-REP step. Briefly, on day 0 of a STD pre-REP step, the tumor tissue fragments are transferred to G-Rex10 plate. Each well is added with 5-30 tumor tissue fragments in 10 to 40 mL of culture medium comprising RPMI, 10% FBS, gentamicin, and 6000 IU/mL of IL-2. The tumor tissue fragments are then cultured at 37° C. in 5% CO2. On Day 5 and Day 10 of the normal pre-REP step, half medium is removed and replaced with fresh medium and 6000 IU/mL of IL-2. After Day 10, half medium is changed every 2 to 3 days. Certain types of immune cells are sorted from the tumor tissue fragments for REP step. Protocol for REP step is described previously in the present disclosure.

In one aspect, the present disclosure provides a composition for treating a tumor comprising in vitro isolated immune cells or reprogrammed immune cells, wherein the immune cells comprise tumor infiltrating lymphocytes expressing IFNγ, CD107a, or HLA-DR, wherein at least 5% of the tumor infiltrating lymphocyte population express IFNγ. In some embodiments, 20% of the tumor infiltrating lymphocyte population express IFNγ. In some embodiments, 5% to 20% of the tumor infiltrating lymphocyte population express IFNγ. In some embodiments, at least 3% of the tumor infiltrating lymphocyte population express CD107a. In some embodiments, 20% of the tumor infiltrating lymphocyte population express CD107a. In some embodiments, 3% to 20% of the tumor infiltrating lymphocyte population express CD107a.

In some embodiments, the immune cells are derived from one or more tumor samples obtained from a subject. In some embodiments, the subject comprises a human, cow, dog, mouse, rat, rabbit, guinea pig, chicken, fish, bird, reptile, camelid, bovine, chimpanzee, sheep, goat, and non-human primate. In some embodiments, the subject is a human.

In some embodiments, at least about 10% of the tumor infiltrating lymphocyte population express the HLA-DR. In some embodiments, at least about 65% of the tumor infiltrating lymphocyte population express the HLA-DR.

In some embodiments, the expanded immune cells, e.g., TILs, or reprogrammed immune cells, e.g., rALI TILs, obtained from the systems and methods described herein are assayed for functional activity. In some embodiments, the assay for functional activity comprises T cell cytotoxicity assays, IL-2 response, etc., as known in the art. In some embodiments, the immune cells, e.g., TILs, or reprogrammed immune cells, e.g., rALI TILs, are assessed for the presence of markers indicative of activation, e.g. expression of CD3, CD25, CD69, CD137, CD107a, Granzyme B (GZMB), Perform 1 (PRF1); etc. In some embodiments, the immune cells, e.g., TILs, are assessed for the HLA-DR, PD-1, CD45, or EPCAM markers. In some embodiments, the immune cells, e.g., TILs, or reprogrammed immune cells, e.g., rALI TILs, can be selected for an activated phenotype prior to administration to a patient or for other applications. In some embodiments, the immune cells, e.g., TILs, or reprogrammed immune cells, e.g., rALI TILs, comprises T cells.

### Treatment

In some instances, a subject in need of a treatment according to the method described herein may be a subject in need of adoptive cellular therapy to treat the subject for cancer or tumor. In some embodiments, the immune cells, e.g., TILs, or reprogrammed immune cells, e.g., rALI TILs, are used for adoptive cellular therapy to treat the subject for cancer or tumor. In some embodiments, the cancer or tumor comprises glioblastoma, colon tumor, lung tumor, gastric tumor, brain tumor, kidney tumor, esophageal tumor, uterine tumor, skin tumor, pancreatic tumor, or breast tumor. In some embodiments, the cancer or tumor is glioblastoma.

In some embodiment, a subject is treated using adoptive cellular therapy employing an expanded and/or reprogrammed cell population that has been activated, expanded, and/or reprogrammed by the methods and/or systems disclosed herein. For example, cells may be collected from a subject, activated and expanded, and reintroduced into the subject as part of the adoptive cellular therapy. In some embodiments, the cells are further reprogrammed to have stem cell-like properties prior to reintroduce into the subject. The cells collected from the subject may be collected from any convenient and appropriate source for the adoptive cellular therapy, such as peripheral blood (e.g., the subject's peripheral blood), a biopsy (e.g., a tumor biopsy from the subject), and the like.

In some embodiments, the cells collected are immune cells. In some embodiments, the immune cells comprise tumor infiltrating lymphocytes (TILs), e.g., TILs collected from a tumor of a subject. In some embodiments, the cells collected are blood cells, e.g., NK cells collected from a subject's blood (e.g., a subject having cancer or a subject having an infection).

In one aspect, the present disclosure provides a therapeutic method comprising introducing into a recipient in need thereof of an expanded cell population, e.g., immune cells, as described above. In some embodiments, the expanded cell population, e.g., immune cells, are reprogrammed to have stem cell-like properties, as described above. In some embodiments, the cell population is autologous or allogeneic with respect to the recipient.

In some embodiments, the expanded immune cells, e.g., TILs, or reprogrammed immune cells, e.g., rALI TILs, can be provided in pharmaceutical compositions suitable for therapeutic use, e.g., for human treatment. In some embodiments, therapeutic formulations comprising the expanded immune cells or reprogrammed immune cells can be frozen, or prepared for administration with physiologically acceptable carriers, excipients or stabilizers (Remington's Pharmaceutical Sciences 16th edition, Osol, A. Ed. (1980), in the form of aqueous solutions. In some instances, the expanded immune cells or reprogrammed immune cells are formulated, dosed, and administered in a fashion consistent with good medical practice. Factors for consideration in this context comprises the particular disorder being treated, the particular mammal being treated, the clinical condition of the individual patient, the cause of the disorder, the site of delivery of the agent, the method of administration, the scheduling of administration, and other factors known to medical practitioners.

In some instances, the expanded immune cells or reprogrammed immune cells can be administered by any suitable means. In some embodiments, the administration comprises intramuscular, intravenous (bolus or slow drip), intraarterial, intraperitoneal, intrathecal, intratumoral, intravesical, or subcutaneous administration. In some embodiments, the administration comprises parenteral infusion. In some embodiments, the expanded immune cells or reprogrammed immune cells can be administered via parenteral infusions. In some embodiments, the parenteral infusions comprise intramuscular, intravenous (bolus or slow drip), intraarterial, intraperitoneal, intrathecal, intratumoral, intravesical, or subcutaneous administration.

In one aspect, the present disclosure provides a composition for treating a tumor comprising in vitro isolated immune cells, wherein the immune cells comprise tumor infiltrating lymphocytes expressing IFNγ, CD107a, or HLA-DR, wherein at least 5% of the tumor infiltrating lymphocyte population express IFNγ. In some instances, the tumor includes, but not are limited to, Colon Tumor, Lung Tumor, Gastric Tumor, Atypical Teratoid/Rhabdoid Tumor, Brain Tumors (e.g., Astrocytomas, Central Nervous System Embryonal Tumors, Central Nervous System Germ Cell Tumors, Craniopharyngioma, Ependymoma, Glioblastoma multiforme (GBM), etc.), Bronchial Tumors, Carcinoid Tumor (e.g., Childhood, Gastrointestinal, etc.), Cardiac (Heart) Tumors, Central Nervous System (e.g., Atypical Teratoid/Rhabdoid Tumor, Embryonal Tumors, Germ Cell Tumor, Lymphoma, etc.), Embryonal Tumors, Extracranial Germ Cell Tumor, Extragonadal Germ Cell Tumor, Islet Cell Tumors (e.g., Pancreatic Neuroendocrine Tumors, etc.), Kidney Cancer (e.g., Renal Cell, Wilms Tumor, Childhood Kidney Tumors, etc.), Ovarian Cancer (e.g., Epithelial, Germ Cell Tumor, Low Malignant Potential Tumor, etc.), Pancreatic Cancer, Pancreatic Neuroendocrine Tumors (Islet Cell Tumors), Pituitary Tumor, Uterine Tumors, Esophageal Tumors, Breast Tumors, Skin Tumors, Liver Tumors, and the like. In some embodiments, as described herein, the tumor infiltrating lymphocytes (TILs) are reprogrammed TILs to have stem cell-like properties.

In some instances, the composition for administration depends on the formulation desired, pharmaceutically-acceptable, non-toxic carriers or diluents, which are defined as vehicles commonly used to formulate pharmaceutical compositions for animal or human administration. In some instances, the diluent is selected and not affect the biological activity of the combination. Examples of diluents are distilled water, physiological phosphate-buffered saline, Ringer's solutions, dextrose solution, and Hank's solution. In some embodiments, the pharmaceutical composition or formulation can comprise other carriers, adjuvants, or nontoxic, nontherapeutic, nonimmunogenic stabilizers and the like.

In some embodiments, the composition further comprises acceptable carriers, excipients, or stabilizers. Acceptable carriers, excipients, or stabilizers are non-toxic to recipients at the dosages and concentrations employed. In some embodiments, acceptable carriers, excipients, or stabilizers comprise buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); non-ionic surfactants such as TWEEN™, PLURONICS™ or polyethylene glycol (PEG); and/or solvent or cryoprotectant such as Dimethylsulfoxide (DMSO).

In some instances, the compositions are prepared as injectables, either as liquid solutions or suspensions. In some instances, the composition in solid forms suitable for solution in, or suspension in, liquid vehicles prior to injection can also be prepared. In some embodiments, proteins can be administered in the form of a depot injection or implant preparation which can be formulated in such a manner as to permit a sustained or pulsatile release of the active ingredient. The pharmaceutical compositions are formulated as sterile, substantially isotonic and in full compliance with all Good Manufacturing Practice (GMP) regulations of the U.S. Food and Drug Administration.

In some instances, once expanded, an effective dose of the immune cells, e.g., TILs or rALI TILs, can then be administered to a patient, including without limitation the patient from which the PDOs were derived from. In some embodiments, the effective dose can be at least about 10² cells, at least about 10³ cells, at least about 10⁴ cells, at least about 10¹⁰ cells, at least about 10⁶ cells, at least about 10⁷ cells, at least about 10⁸ cells, at least about 10⁹ cells, at least about 10¹⁰ cells, at least about 10¹¹ cells, at least about 10¹² cells, at least about 10¹³ cells, or more per administration. In some embodiments, the effective dose can be at least about 10² cells to about 10¹³ cells, at least about 10³ cells to about 10¹³ cells, at least about 10⁴ cells to about 10¹³ cells, at least about 10¹⁰ cells to about 10¹³ cells, at least about 10⁶ cells to about 10¹³ cells, at least about 10⁷ cells to about 10¹³ cells, at least about 10⁸ cells to about 10¹³ cells, at least about 10⁹ cells to about 10¹³ cells, at least about 10¹⁰ cells to about 10¹³ cells, at least about 10¹¹ cells to about 10¹³ cells, or at least about 10¹² cells to about 10¹³ cells per administration. In some embodiments, the effective dose can be at least about 10¹² cells to about 10¹¹ cells, at least about 10³ cells to about 10¹¹ cells, at least about 10⁴ cells to about 10¹¹ cells, at least about 10¹⁰ cells to about 10¹¹ cells, at least about 10⁶ cells to about 10¹¹ cells, at least about 10⁷ cells to about 10¹¹ cells, at least about 10⁸ cells to about 10¹¹ cells, at least about 10⁹ cells to about 10¹¹ cells, or at least about 10¹⁰ cells to about 10¹¹ cells per administration. In some embodiments, the effective dose can be delivered systemically, by intratumoral injection or other administrative routes as described above.

In some instances, following the administration, an enhanced immune response may be manifest as an increase in the cytolytic response of T cells towards the target cells present in the recipient.

In some embodiments, patient who will receive the expanded immune cells may also receive chemotherapy.

In some embodiments, the composition further comprises acceptable carriers, excipients, or stabilizers. In some embodiments, the composition can be administered with other therapeutic treatments. In some embodiments, the other therapeutic treatments comprise chemotherapeutic agents, immune checkpoint inhibitors, cancer therapeutics, targeted therapeutics, immunomodulators, cytokines, antibiotics, or antiviral agents.

### E. Combination Therapy

In one aspect, the present disclosure provides composition that can be administered with other therapeutic treatments. In some embodiments, the other therapeutic treatments comprise chemotherapeutic agents, immune checkpoint inhibitors, cancer therapeutics, targeted therapeutics, immunomodulators, cytokines, antibiotics, or antiviral agents. In some instances, cancer cells prevent immune cells from attacking by braking in the immune system or using signals from the tumor that weaken the immune response. In some instances, immune checkpoint inhibitors are utilized along with adoptive cellular therapy. In some embodiments, the expanded immune cells or reprogrammed immune cells are treated with immune checkpoint inhibitors prior to administration to the patient.

In some embodiments, treatment of a subject or patient for a condition employing a composition and/or cells of the subject disclosure can be combined with one or more additional active agents. In some instances, useful additional active agents comprise active agents for treating cancer.

In some embodiments, treatment can be combined with other active agents, including antibiotics, cytokines, and antiviral agents. In some embodiments, antibiotics comprises penicillins, e.g. penicillin G, penicillin V, methicillin, oxacillin, carbenicillin, nafcillin, ampicillin, etc. In some embodiments, the penicillins are used in combination with β-lactamase inhibitors, cephalosporins, e.g. cefaclor, cefazolin, cefuroxime, moxalactam, etc.; carbapenems; monobactams; aminoglycosides; tetracyclines; macrolides; lincomycins; polymyxins; sulfonamides; quinolones; cloramphenical; metronidazole; spectinomycin; trimethoprim; vancomycin; etc. In some embodiments, the cytokines comprise interferon γ, tumor necrosis factor α, interleukin 12, etc. In some embodiments, the antiviral agents comprises acyclovir, gancyclovir, etc.

In some instances, where treatment is directed to cancer, chemotherapeutic agents that can be administered in combination with the expanded immune cells or reprogrammed immune cells. In some embodiments, the chemotherapeutic agents comprise abitrexate, adriamycin, adrucil, amsacrine, asparaginase, anthracyclines, azacitidine, azathioprine, bicnu, blenoxane, busulfan, bleomycin, camptosar, camptothecins, carboplatin, carmustine, cerubidine, chlorambucil, cisplatin, cladribine, cosmegen, cytarabine, cytosar, cyclophosphamide, cytoxan, dactinomycin, docetaxel, doxorubicin, daunorubicin, ellence, elspar, epirubicin, etoposide, fludarabine, fluorouracil, fludara, gemcitabine, gemzar, hycamtin, hydroxyurea, hydrea, idamycin, idarubicin, ifosfamide, ifex, irinotecan, lanvis, leukeran, leustatin, matulane, mechlorethamine, mercaptopurine, methotrexate, mitomycin, mitoxantrone, mithramycin, mutamycin, myleran, mylosar, navelbine, nipent, novantrone, oncovin, oxaliplatin, paclitaxel, paraplatin, pentostatin, platinol, plicamycin, procarbazine, purinethol, ralitrexed, taxotere, taxol, teniposide, thioguanine, tomudex, topotecan, valrubicin, velban, vepesid, vinblastine, vindesine, vincristine, vinorelbine, VP-16, and vumon.

In some embodiments, targeted therapeutics that can be administered in combination with the expanded immune cells or reprogrammed immune cells comprise tyrosine-kinase inhibitors, such as Imatinib mesylate (Gleevec, also known as STI-571), Gefitinib (Iressa, also known as ZD1839), Erlotinib (marketed as Tarceva), Sorafenib (Nexavar), Sunitinib (Sutent), Dasatinib (Sprycel), Lapatinib (Tykerb), Nilotinib (Tasigna), and Bortezomib (Velcade); Janus kinase inhibitors, such as tofacitinib; ALK inhibitors, such as crizotinib; Bcl-2 inhibitors, such as obatoclax, venclexta, and gossypol; FLT3 inhibitors, such as midostaurin (Rydapt), IDH inhibitors, such as AG-221, PARP inhibitors, such as Iniparib and Olaparib; PI3K inhibitors, such as perifosine; VEGF Receptor 2 inhibitors, such as Apatinib; AN-152 (AEZS-108) doxorubicin linked to [D-Lys(6)]-LHRH; Braf inhibitors, such as vemurafenib, dabrafenib, and LGX818; MEK inhibitors, such as trametinib; CDK inhibitors, such as PD-0332991 and LEE011; Hsp90 inhibitors, such as salinomycin; and/or small molecule drug conjugates, such as Vintafolide; serine/threonine kinase inhibitors, such as Temsirolimus (Torisel), Everolimus (Afinitor), Vemurafenib (Zelboraf), Trametinib (Mekinist), and Dabrafenib (Tafinlar).

In some embodiments, the expanded immune cells or reprogrammed immune cells can be administered in combination with an immunomodulator, such as a cytokine, a lymphokine, a monokine, a stem cell growth factor, a lymphotoxin (LT), a hematopoietic factor, a colony stimulating factor (CSF), an interferon (IFN), a transforming growth factor (TGF), such as TGF-α or TGF-β, insulin-like growth factor (IGF), erythropoietin, thrombopoietin, a tumor necrosis factor (TNF) such as TNF-α or TNF-β, vascular endothelial growth factor, integrin, granulocyte-colony stimulating factor (G-CSF), granulocyte macrophage-colony stimulating factor (GM-CSF), an interferon such as interferon-α, interferon-β, or interferon-γ, Si factor, an interleukin (IL) such as IL-1, IL-1cc, IL-2, IL-3, IL-4, IL-5, IL-6, IL-7, IL-8, IL-9, IL-10, IL-11, IL-12, IL-13, IL-14, IL-15, IL-16, IL-17, IL-18 IL-21 or IL-25, LIF, kit-ligand, FLT-3, endostatin, and LT.

In some embodiments, tumor specific monoclonal antibodies that can be administered in combination with the expanded immune cells or reprogrammed immune cells comprise Ipilimumab targeting CTLA-4 (as used in the treatment of Melanoma, Prostate Cancer, RCC); Tremelimumab targeting CTLA-4 (as used in the treatment of CRC, Gastric, Melanoma, NSCLC); Nivolumab targeting PD-1 (as used in the treatment of Melanoma, NSCLC, RCC); MK-3475 targeting PD-1 (as used in the treatment of Melanoma); Pidilizumab targeting PD-1 (as used in the treatment of Hematologic Malignancies); BMS-936559 targeting PD-L1 (as used in the treatment of Melanoma, NSCLC, Ovarian, RCC); MEDI4736 targeting PD-L1; MPDL33280A targeting PD-L1 (as used in the treatment of Melanoma); Rituximab targeting CD20 (as used in the treatment of Non-Hodgkin's lymphoma); Ibritumomab tiuxetan and tositumomab (as used in the treatment of Lymphoma); Brentuximab vedotin targeting CD30 (as used in the treatment of Hodgkin's lymphoma); Gemtuzumab ozogamicin targeting CD33 (as used in the treatment of Acute myelogenous leukaemia); Alemtuzumab targeting CD52 (as used in the treatment of Chronic lymphocytic leukaemia); IGN101 and adecatumumab targeting EpCAM (as used in the treatment of Epithelial tumors (breast, colon and lung)); Labetuzumab targeting CEA (as used in the treatment of Breast, colon and lung tumors); huA33 targeting gpA33 (as used in the treatment of Colorectal carcinoma); Pemtumomab and oregovomab targeting Mucins (as used in the treatment of Breast, colon, lung and ovarian tumors); CC49 (minretumomab) targeting TAG-72 (as used in the treatment of Breast, colon and lung tumors); cG250 targeting CAIX (as used in the treatment of Renal cell carcinoma); J591 targeting PSMA (as used in the treatment of Prostate carcinoma); MOv18 and MORAb-003 (farletuzumab) targeting Folate-binding protein (as used in the treatment of Ovarian tumors); 3F8, chi4.18 and KW-2871 targeting Gangliosides (such as GD2, GD3 and GM2) (as used in the treatment of Neuroectodermal tumors and some epithelial tumors); hu3S193 and IgN311 targeting Le y (as used in the treatment of Breast, colon, lung and prostate tumors); Bevacizumab targeting VEGF (as used in the treatment of Tumor vasculature); IM-2C6 and CDP791 targeting VEGFR (as used in the treatment of Epithelium-derived solid tumors); Etaracizumab targeting Integrin V_3 (as used in the treatment of Tumor vasculature); Volociximab targeting Integrin 51 (as used in the treatment of Tumor vasculature); Cetuximab, panitumumab, nimotuzumab and 806 targeting EGFR (as used in the treatment of Glioma, lung, breast, colon, and head and neck tumors); Trastuzumab and pertuzumab targeting ERBB2 (as used in the treatment of Breast, colon, lung, ovarian and prostate tumors); MM-121 targeting ERBB3 (as used in the treatment of Breast, colon, lung, ovarian and prostate, tumors); AMG 102, METMAB and SCH 900105 targeting MET (as used in the treatment of Breast, ovary and lung tumors); AVE1642, IMC-A12, MK-0646, R1507 and CP 751871 targeting IGF1R (as used in the treatment of Glioma, lung, breast, head and neck, prostate and thyroid cancer); KB004 and IIIA4 targeting EPHA3 (as used in the treatment of Lung, kidney and colon tumors, melanoma, glioma and haematological malignancies); Mapatumumab (HGS-ETR1) targeting TRAILR1 (as used in the treatment of Colon, lung and pancreas tumors and haematological malignancies); HGS-ETR2 and CS-1008 targeting TRAILR2; Denosumab targeting RANKL (as used in the treatment of Prostate cancer and bone metastases); Sibrotuzumab and F19 targeting FAP (as used in the treatment of Colon, breast, lung, pancreas, and head and neck tumors); 81C6 targeting Tenascin (as used in the treatment of Glioma, breast and prostate tumors); Blinatumomab (Blincyto; Amgen) targeting CD3 (as used in the treatment of ALL); pembrolizumab targeting PD-I as used in cancer immunotherapy; 9E10 antibody targeting c-Myc; and etc.

### Other Applications of Expanded Immune Cells or Reprogrammed Immune Cells

In one aspect, the expanded immune cells, e.g., TILs, or reprogrammed immune cells, e.g., rALI TILs, as described in the present disclosure can be used for other applications aside from immunotherapy. In some embodiments, the expanded immune cells, e.g., TILs, or reprogrammed immune cells, e.g., rALI TILs, can be used for functional in vitro assays for disease modeling or for determining patient specific responsiveness to immunotherapy agent. In some embodiments, the expanded immune cells, e.g., TILs, or reprogrammed immune cells, e.g., rALI TILs, can be used for screening assays. In some embodiments, the expanded immune cells, e.g., TILs, or reprogrammed immune cells, e.g., rALI TILs, can be used for determining preclinical efficacy of immunotherapy agent. In some embodiments, the expanded immune cells, e.g., TILs, or reprogrammed immune cells, e.g., rALI TILs, can be used for various applications/experiments modified from methods known in the art.

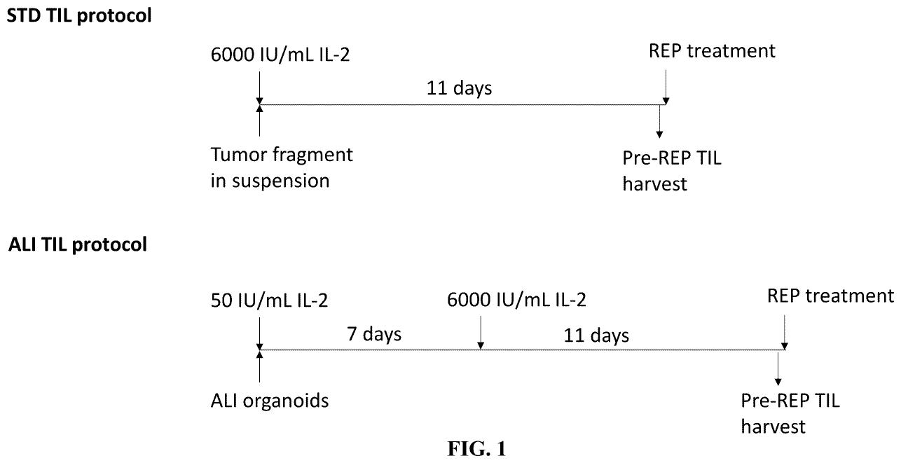

FIG. 1 depicts the timeline of pre-Rapid Expansion Protocol (pre-REP) in Standard Tumor Infiltrating Lymphocytes (STD TIL) protocol and in air-liquid interface Tumor Infiltrating Lymphocytes (ALI TIL) protocol. Standard (“STD”) refers to immune cells obtained from a method involving obtaining and culturing TIL from fresh tumor tissue and treated with one concentration of cytokine during the pre-REP step. Briefly, on Day 0 of the STD pre-REP step, the tumor tissue fragments are transferred to G-Rex10 plate. Each well is added with 5-30 tumor tissue fragments in 10 to 40 mL of culture medium comprising RPMI, 10% FBS, gentamicin, and 6000 IU/mL of IL-2. The tumor tissue fragments are then cultured at 37° C. in 5% CO₂. On Day 5 and Day 10 of the STD pre-REP step, half medium is removed and replaced with fresh medium and 6000 IU/mL of IL-2. After Day 10, half medium is changed every 2 to 3 days. At the end of day 11, cells are collected before being treated with cocktails for Rapid Expansion Protocol (REP) treatment. Certain types of immune cells are sorted from the tumor tissue fragments for REP step. Protocol for REP step is described previously in the present disclosure.

In contrast to STD TIL protocol, as described in this present disclosure, in ALI TIL protocol, the starting samples are patient derived organoids (PDOs), which are generated from tissue samples. These PDOs are cultured in air-liquid interface (ALI) environment. These PDOs comprise various immune populations, e.g., tumor infiltrating immune cells, lymphocytes, T cells, etc., which can then be used for expansion. In the pre-REP phase of ALI TIL protocol, the PDOs or ALI organoids are treated with 50 IU/mL of IL-2 for 1-2 weeks before being treated with higher concentrations of IL-2 (6000 IU/mL) for 11 days. At the end of this, cells are harvested for REP treatment.

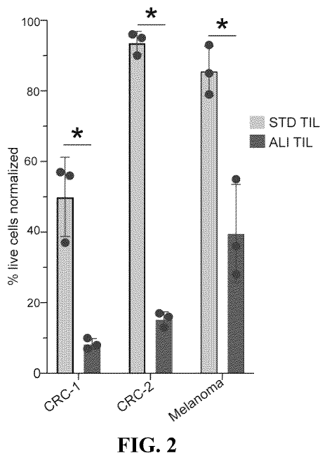

FIG. 2 shows that ALI Tumor Infiltrating Lymphocytes (ALI TILs) demonstrate a better tumor killing capacity compared to standard TILs (STD TILs). ALI TILs and STD TILs were co-culture with autologous tumor epithelial cells derived from submerged Organoids for 48 hours. Single Cell tumor Organoids alone were used as a control. At 48 hours post-incubation, cells were stained with fluorochrome-conjugated antibodies for CD45, EPCAM, Annexin V, Zombie NIR live dead and other T cell markers. Flow Cytometry analysis for live tumor cell count was performed after staining. To assess the tumor killing capacity of TILs, the absolute number of tumor cells in the conditions where autologous tumor organoids were co-cultured with TILs was divided by the absolute number of tumor cells in the conditions where autologous tumor organoids were cultured without TILs (control condition). The result of these analyses is expressed on percentage of live cells. Data is shown for samples derived from 3 different patients (CRC-1=colorectal cancer patient 1, CRC-2=colorectal cancer patient 2, MLN=melanoma patient). 3 replicates per experiment were performed. Nonparametric ANOVA tested corrected by Geisser Greenhouse Correction was performed to compare between different conditions; *≤Pv=0.05.

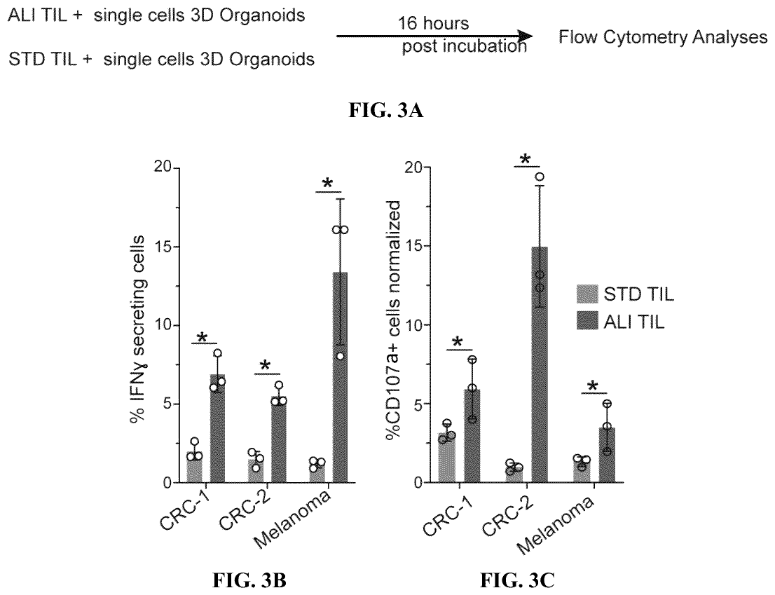

FIGS. 3A-3C show that ALI Tumor Infiltrating Lymphocytes (ALI TILs) are more reactive to tumor cells compared to standard TILs (STD TILs). FIG. 3A shows the experimental scheme. Briefly, ALI TILs and STD TILs were co-cultured with autologous tumor epithelial cells derived from submerged Organoids for 16 hours. ALI TILs or STD TILs alone were used as a control. At 16 hours post-incubation, cells were stained with fluorochrome-conjugated antibodies for IFNγ secretion, CD107a, CD45, EPCAM, Annexin V, Zombie NIR live dead and other T cell markers for Flow Cytometry analysis. Next, Flow Cytometry analysis for live CD3+ cells was performed after staining. In FIG. 3B and FIG. 3C, to assess the tumor-reactive of TILs, the percentage of CD3+ cells secreting IFNγ and the % of CD3+ cells expressing CD107a on their surface were assessed at 16 hours post-incubation. Data is shown for samples derived from 3 different patients (CRC-1=colorectal cancer patient 1, CRC-2=colorectal cancer patient 2, MLN=melanoma patient). 3 replicates per experiment were performed. Nonparametric ANOVA tested corrected by Geisser Greenhouse Correction was performed to compare between different conditions; *≤Pv=0.05.

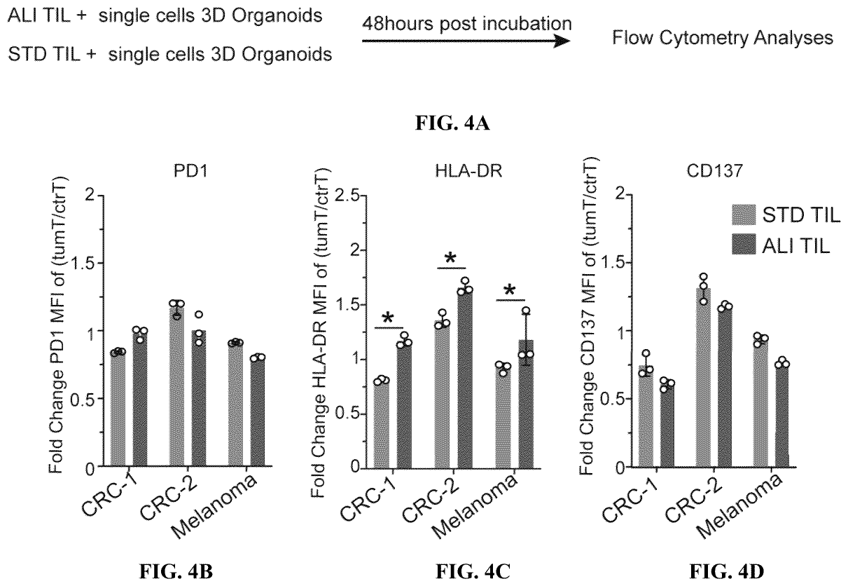

FIGS. 4A-4D show that ALI Tumor Infiltrating Lymphocytes (ALI TILs) express higher levels of HLA-DR compared to standard TILs (STD TILs). FIG. 4A shows the experimental scheme. Briefly, ALI TILs and STD TILs were co-cultured with autologous tumor epithelial cells derived from submerged Organoids for 48 hours. ALI TILs or STD TILs alone were used as a control. 48 hours post-incubation, cells were stained with fluorochrome-conjugated antibodies for HLA-DR, CD137, PD1, CD45, EPCAM, Annexin V, Zombie NIR live dead and other T cell markers for Flow Cytometry analysis. Next, Flow Cytometry analysis for live CD3+ cells was performed after staining. In FIGS. 4B-4D, in order to assess the tumor-reactive of TILs, the MFI (Median Fluorescence Intensity) of PD1, HLA-DR and CD137 was quantified and compared to the MFI of ALI TILs or STD TILs that were not exposed to tumor cells. The result is presented on Fold Change. Data is shown for samples derived from 3 different patients (CRC-1=colorectal cancer patient 1, CRC-2=colorectal cancer patient 2, MLN=melanoma patient). 3 replicates per experiment were performed. Nonparametric ANOVA tested corrected by Geisser Greenhouse Correction was performed to compare between different conditions; *≤Pv=0.05

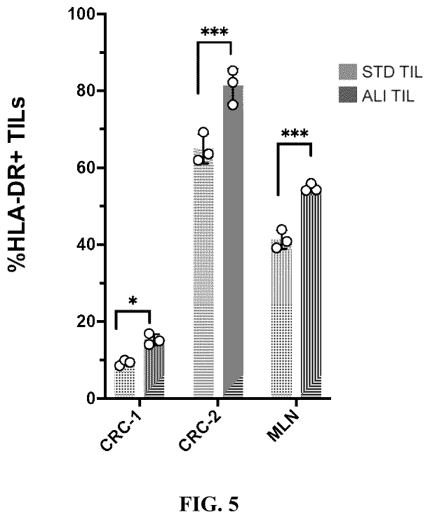

FIG. 5 shows that ALI Tumor Infiltrating Lymphocytes (ALI TILs) express higher percentage of HLA-DR+ compared to standard TILs (STD TILs). The experimental timeframe and condition were similar as described in FIGS. 4A-4D. The result is presented in percentage instead of Fold Change as in FIG. 4C. Data is shown for samples derived from 3 different patients (CRC-1=colorectal cancer patient 1, CRC-2=colorectal cancer patient 2, MLN=melanoma patient). 3 replicates per experiment were performed. Nonparametric ANOVA tested corrected by Geisser Greenhouse Correction was performed to compare between different conditions; *≤Pv=0.05

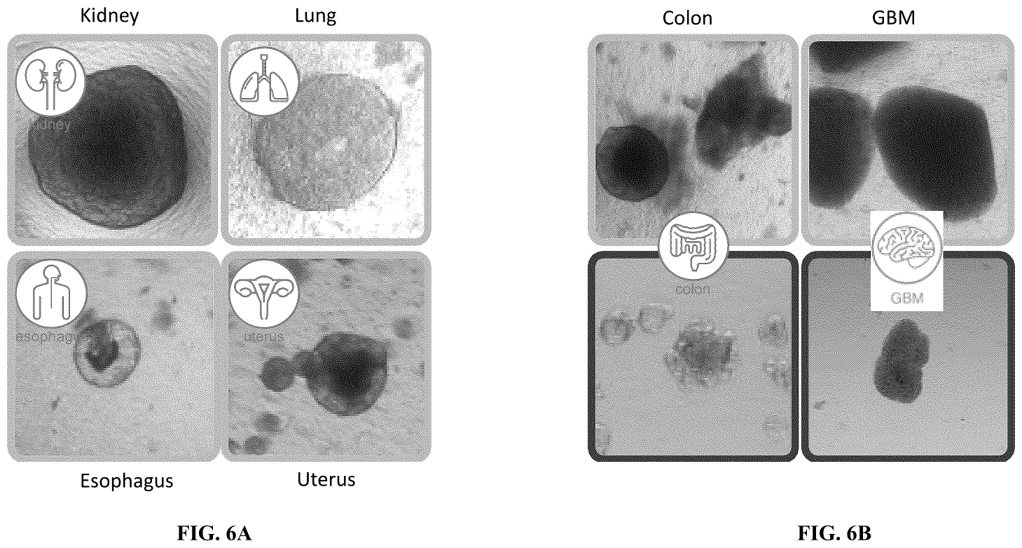

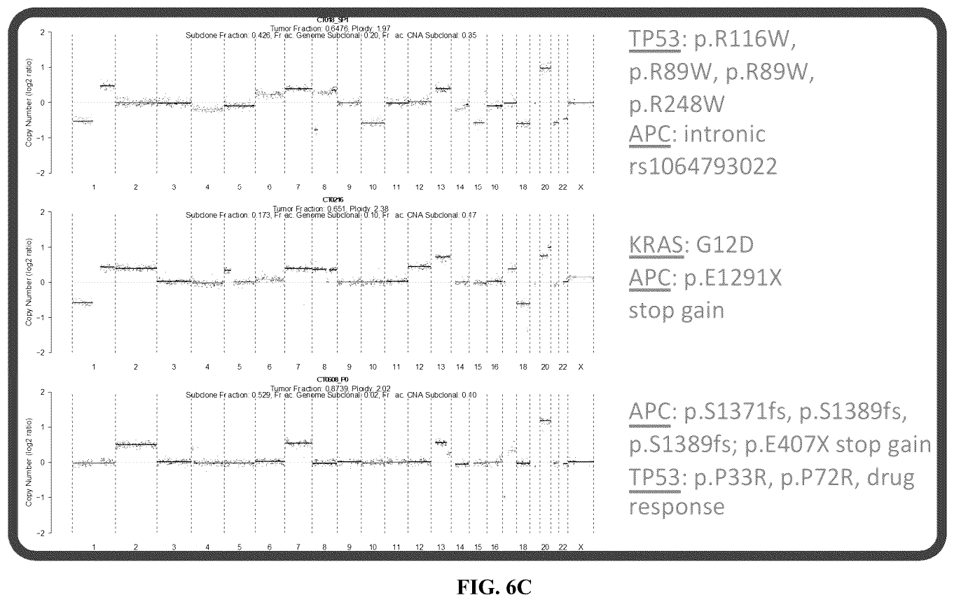

FIGS. 6A-6C illustrate an example of the establishment of ALI tumor organoids. FIG. 6A shows representative brightfield images of ALI tumor organoids generated from tissue obtained from kidney, lung, esophagus, and uterus. FIG. 6B shows an example of ALI tumor organoids generated from tissue obtained from colon and glioblastoma (GBM). Top figures show samples grown as air-liquid interface (ALI) organoids, which can be used for TIL expansion and/or reprogramming. Bottom figures show submerged organoids, which can be used for assays. FIG. 6C shows sequencing results for three colorectal cancer (CRC) organoid lines, which are shown as CNV plots and mutated genes (APC, TP53, KRAS).

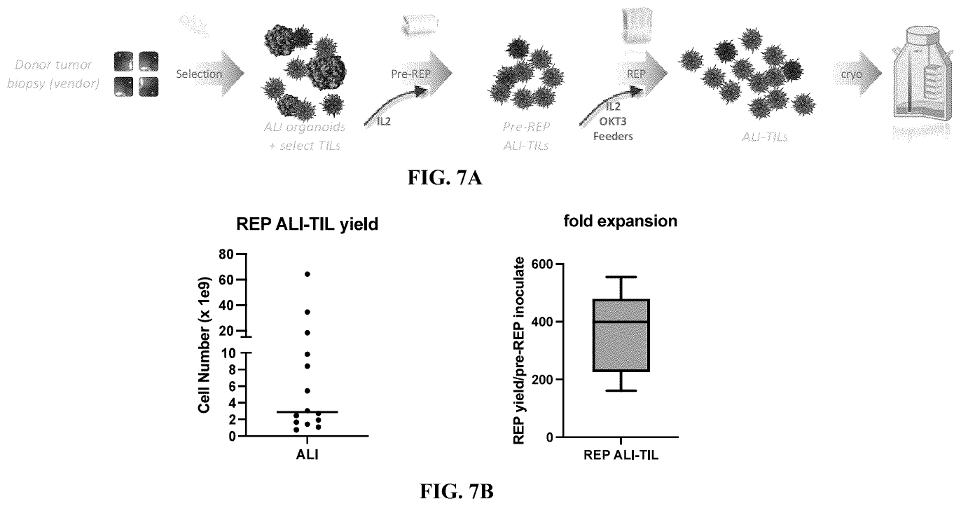

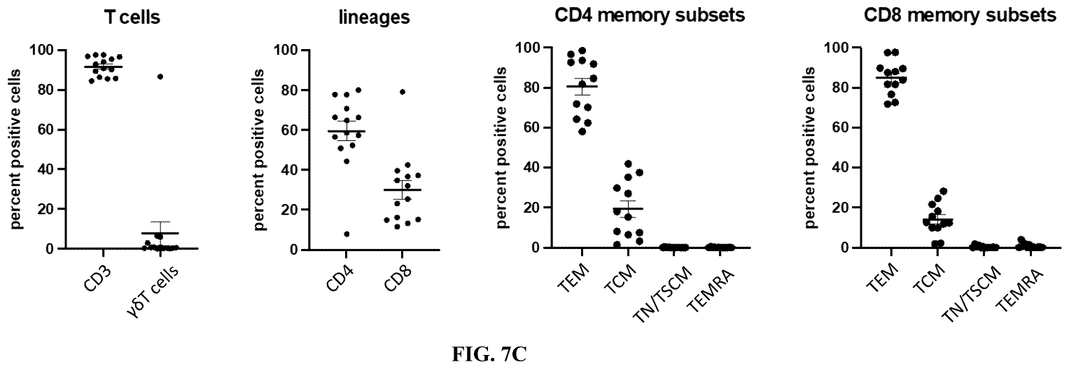

FIGS. 7A-7C demonstrate the generation and phenotypic characterization of ALI TILs. FIG. 7A depicts a schematic representation of the 2-step ALI TIL process and cryopreservation of the product comprising ALI TILs. FIG. 7B shows cell number of ALI TILs obtained from 14 preps (left) and the fold-expansion distribution of the 14 products (right). FIG. 7C shows results of flow cytometry analysis using CD3, γδTCR, CD4, CD8, CD45RA, and CD62L to detect T cells, lineage, and memory subsets of twelve ALI-TIL preps. Results are plotted as percent parent for each individual sample with average and SEM. (TEM: effector memory T cells; TCM: central memory T cells; TN/TSCM: stem cell memory T cells; TEMRA: terminally differentiated effector memory cells)

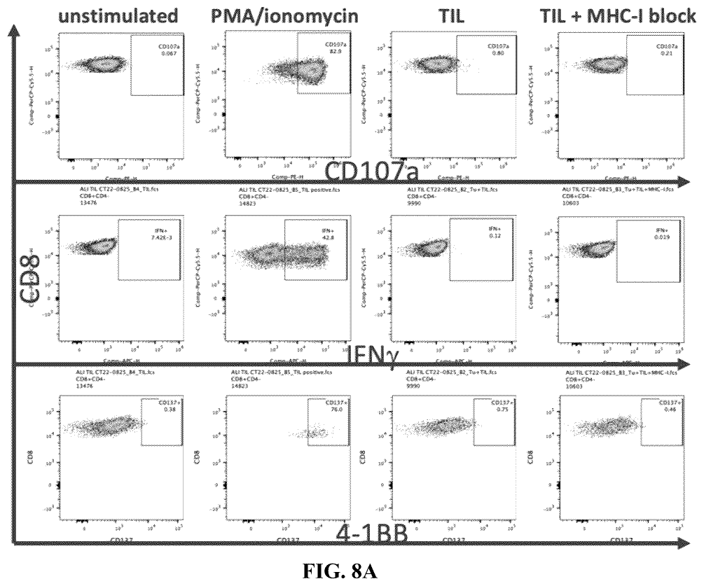

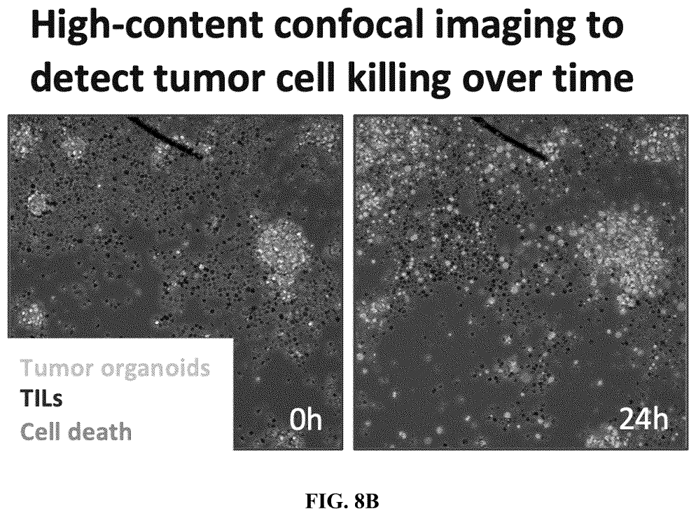

FIGS. 8A and 8B show ALI TIL tumor reactivity and cytotoxicity detection. FIG. 8A shows flow cytometry analysis of ALI TIL from CRC co-cultured with autologous tumor organoids for 16 hours at 1:1 effector:target ratio. Increased IFNγ, CD107a, and 4-1BB were observed. PMA/ionomycin was used as activation control and W2/36 to block MHCI presentation and confirm antigen specificity. Cells were stained for live/dead, EPCAM, CD3, CD8, and CD107a & IFNγ or 4-1BB and analyzed by flow, using a Quanteon instrument (Agilent, Santa Clara, CA). Data were processed with FlowJo (Ashland, OR), gating for indicated marker as shown. FIG. 8B shows results obtained from confocal imaging at 0 hour (0h) and 24 hours (24h) of co-culturing experiment of ALI TILs obtained from CRC and autologous tumor organoids for 24 hours at a 10:1 effector:target ratio. Tumor cell killing was observed over time.

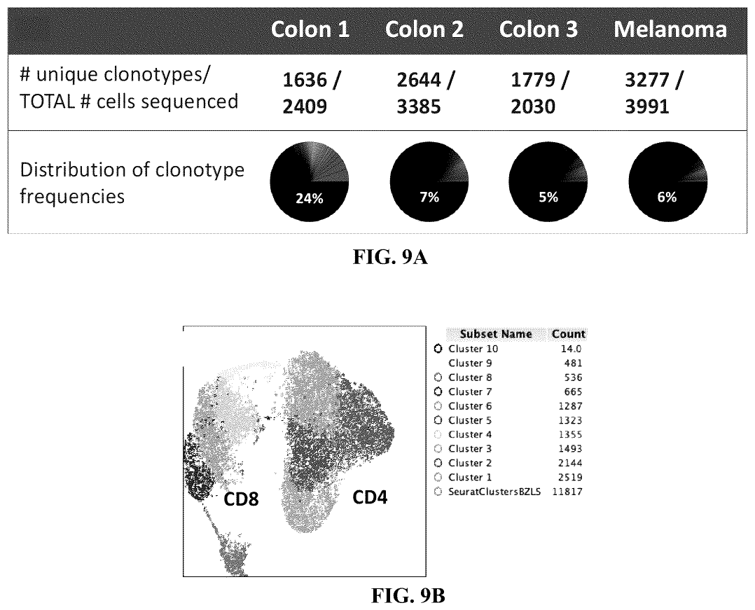

FIGS. 9A and 9B display the results of single-cell RNA seq analysis of CRC and melanoma ALI-TILs. FIG. 9A shows numbers of unique clonotypes relative to number of sequenced cells (upper row) and distribution of clonotype frequencies (lower row). Unique paired αβ CDR3 sequences (assimilated to TCR clonotypes) were counted and ranked by order of frequencies. FIG. 9B shows expression levels of 397 immune genes which identify 10 clusters using SeqGeq.

### EXAMPLES

The following examples are included for illustrative purposes only and are not intended to limit the scope of the invention.

### Example 1: Air-Liquid Interface Tumor Infiltrating Lymphocytes (ALI TILs) Demonstrated a Better Tumor Killing Capacity Compared to Standard Tumor Infiltrating Lymphocytes (STD TILs)

In order to assess tumor killing capacity of the air-liquid interface (ALI) generated tumor infiltrating lymphocytes (TILs) as described in the present disclosure, the ALI culture and expansion methods were performed.

TABLE 1
Materials and reagents for PDO culture establishment in ALI
environment and pre-rapid expansion protocol TILs.

| Items | Supplier | Catalog Number |
|---|---|---|
| DMEM | Life Technologies | 11995-073 |
| FBS | Life Technologies | 26140079 |
| ADMEM | Life Technologies | 12634-028 |
| PSQ | Life Technologies | 10378016 |
| HEPES | Life Technologies | 15630-080 |
| Glutamax | Life Technologies | 35050-061 |
| NAC | Sigma | A9165-100G |
| B-27 | Life Technologies | 125870-01 |
| A83-01 | Tocris | 2939 |
| Gastrin | Sigma | G9145-.5MG |
| Normocin | Invivogen | ant-nr-1 |
| EGF | Peprotech | AF-100-15 |
| Noggin | Peprotech | 120-10C |
| FGF-10 | Peprotech | 100-26 |
| SB202190 | Biogems | 1523072 |
| NIC | Sigma | N0636-500G |
| Y27632 | Peprotech | 1293823 |
| RPMI 1640 L-glutamine | Cytiva | SH30027.FS |
| Millicell Culture Dish | Fisher Scientific | PICM03050 |
| Insert | | |
| 555646S IRIS SCISSORS | Prome Biosciences | 555646S |
| 11CM TITANIUM | | |
| HypoThermosol ® FRS | Stemcell technologies | 7936 |
| Hardened Fine Scissors | Fine Sci Tools | 14090-11 |
| CELLMATRIX | FUJIFILM Wako | 637-00653 |
| TYPE I-A, | Chemicals U.S.A. | |
| | Corporation | |
| Collagenase Type 4 | Worthington | LS004212 |
| 5×50 mg | | |
| Corning ® cell lifter | Sigma | CLS3008-100EA |
| Normocin | InvivoGen | # an-nr-1 |
| PBS without calcium, | Fisher Scientific | MT-21-040-CV |
| magnesium | | |

Establishing Patient-Derived Organoids (PDO) or ALI Organoids from prnmMr Tumor Tissues in 3D ALI Environment

### Preparation of Culture Inserts and Collagen Before Handling the Tissue

In this step, collagen liquid mixture was prepared according to the manufacturer's instructions. The collagen liquid mixture was prepared and kept on ice. Next, 1 mL of collagen liquid mixture was added into the 0.4.m insert. The insert containing collagen mixture was then left at room temperature for at least 30 min to solidify. The rest of collagen liquid mixture was kept on ice until later use in organoid culture step.

The numbers of inserts are prepared according to the size of the tissue sample. Total volume per insert is 2 mL.

### Preparation of Organoid Culture

The tissue sample for organoid culture was prepared by mincing the tissue using scissors until the tissue became paste-like texture and almost-liquid form. There were no chunks in this step, and the tissue was kept hydrated with appropriate volume of medium throughout the process. At this step, some of minced tissue can be frozen down in −80° C. Alternatively, all of the tissue or the rest of the tissue can be used for organoid culture.

Next, 5 mL ofF12 medium containing Normocin (F12-Normocin medium) was added to the petri dish to transfer minced tissue to a 15 mL conical tube. The petri dish was then wash with 5-10 mL of F12-Normocin, and all leftover minced tissued was collected into the 15 mL conical tube. The minced tissued was then spin down using a centrifuge at 400×g for 3 min. The medium was then aspirated, and pellet of minced tissue was wash with 10 mL of F12-Normocin medium. The minced tissued was then spin down at 400×g for 3 min. Additional washing step may be required depending on the quality of tissue obtained and the content of the fatty tissue in the sample.

After the washing step, the minced tissue was resuspended and reconstitute in collagen liquid mixture that was previously prepared. Each insert was then added with 1 mL of minced tissue dissolved in collagen liquid mixture directly on top of the collagen matrix-inserts that was previously prepared. The inserts containing minced tissue were then incubated at 37° C. for 30-45 min to allow collagen liquid mixture to solidify.

Next, 1.5 mL of culture medium was added outside of the insert. The plate or outer well holding the insert was shake gently to allow the medium to cover the bottom of the insert.

Prior to establishing the organoid culture, parts of the tissue samples can be kept for histology and/or sequencing experiments later. In order to store the tissue samples, the tissue was washed with 5 times (5×) volume of F12-Normocin in a 10 cm culture dish. Optionally, the tissue can be transferred to a petri dish on ice and cut into smaller pieces for IHC experiments. The tissue can be fixed in 4% PFA or 10% Formalin overnight at 4° C.

### Passage of ALI Organoids

After culturing the ALI organoids for 2 to 4 weeks, ALI organoids can be passaged.

The collagen liquid mixture was prepared as described above and kept on ice. Each 0.4 μm insert was then added with 1 mL of collagen liquid mixture and left at room temperature for 30 min to solidify.

In order to passage the ALI organoids, collagenase solution was prepared by adding 950 μL of F12 medium with 50 μL of Collagenase stock, which was prepared by adding 5 mL of PBS into 1 vial of Collagenase. The tissue in collagen gel insert was collected using a scraper, and each insert was added with 50-100 μL of collagenase solution so each insert was added with 300U of collagenase. The tissue in collagen gel and collagenase solution were then incubated at 37° C. in a gently shaker for 45 min.

During the first wash, 10 mL of ADMEM/F12 medium was added. The tissue was next centrifuged at 600×g for 3 min. Supernatant was the discard, and each tube was added with 1 mL of Matrigel recovery solution (MRS). P1000 pipette was used to break the gel/pellet gently by tritulation.

Next, during the second wash, 9 mL of ADMEM/F12 was added to 10 mL before centrifuging the sample at 600×g for 3 min. The supernatant was then discarded.

During the third wash, 1 mL of ADMEM/F12 was added, and P1000 pipette was used to break gel/pellet by tritulating gently. The sample was then centrifuged at 600×g for 3 min and the supernatant was then discarded.

Next, the pellet was resuspended in collagen liquid mixture. Each insert was then added with 1 mL of pellet/collagen liquid mixture, on top of the collagen layer. The inserts were then left in room temperature for 30 min to solidify.

Each outer well of the insert was then added with 1.5 mL of culture medium. The solidified inserts were then placed in the center of the outer well to ensure that the medium spread and cover the bottom of the insert.

Optionally, the ALI organoids can be cryopreserved. The freezing medium comprising 90% FBS and 10% DMSO was prepared prior cryopreservation. The medium was aspirated from the culture dish. Next, the collagen gel containing ALI organoids was collected using a cell scraper or P1000 pipette tip before transferring into a 15 mL conical tube. The volume of collagenase IV was calculated according to the number of inserts; about 50-100 μL collagenase IV and 500 μL of culture medium were used per insert. The ALI organoids were then shake at 37° C. for 30-60 min until collagen gel was dissolved. Next, 3× volume medium was added to the tube. The tube was then spun at 400×g for 3 min. Supernatant was discarded, and each tube was added with 3 mL of culture medium and ALI organoids were triturated gently using P1000 pipette. Next, 5 mL of culture medium was added to the tube and the tube was spun at 400×g for 3 min. The supernatant was aspirated, and the ALI organoids were then resuspended in the freezing medium. The ALI organoids were mixed gently by pipetting. Each 0.5 mL of suspension was then added to each cryovial. The cryovial was placed in −80° C. for 24 hours before transferring to liquid nitrogen for long term storage.

### Pre-Rapid Expansion Protocol (Pre-REP) of ALI TILs in ALI Organoids

In order to prepare tissues and cells for Rapid Expansion of ALI derived TILs, after establishing organoids from tumor tissue, 50 IU/mL of IL-2 was added to the culture medium. The ALI organoids were then cultured for 7 to 14 days. Between day 7 to day 14, the ALI organoids were collected using a scraper. Collagenase IV was used in this step to collect ALI organoids. Next, to prepare TIL initiation between day 7 to day 14, the organoid pellets were resuspended into one well of G-Rex 24 Well Plate, and 6000 IU/mL of IL-2 in RPMI medium was add to each well. The total volume of each well was about 6 mL. 6000 IU/mL of IL-2 was added to the culture every three days. On day 11 after TIL initiation (or day 18 or day 24 after the start of 50 IU/mL of IL-2 treatment, cells were harvested and were filtered through a 100 μm strainer. Next, cells were spun at 600×g for 3 min. Cells were then ready for REP step. For comparison, standard TILs (STD TILs) from standard pre-REP protocol by culturing with 6000IU/mL IL-2 for 11 days were harvested separately. Alternatively, these cells can be counted and freeze down using freezing medium as described previously.

### Rapid Expansion Protocol (REP) for ALI TILs

TABLE 2
Materials and reagents for REP step of ALI derived TILs

| Materials/reagents | Vendor | Cat |
|---|---|---|
| G-Rex 6M | Wisonwolf | 80660M |
| AIM-V medium | ThermoFisher | 12-055-091 |
| IL-2 | Pepro Tech | 200-02 |
| Anti-CD3 mAb | Takara | T210 |
| RPMI 1640 Medium, GlutaMAX ™ | ThermoFisher | 72400047 |
| Supplement, HEPES | | |
| Gentamicin 50 mg/mL | Sigma | G1397-10 mL |

TABLE 3
Reagents for Complete Medium (CM)

| | Vendor | Cat | Final concen | 500 mL | |
|---|---|---|---|---|---|
| RPMI 1640 Medium, | ThermoFisher | 72400047 | | Bring volume up | |
| GlutaMAX ™ | | | | to 500 mL | |
| Supplement, HEPES | | | | | |
| Human Serum Albumin | Sigma-Aldrich | H4522-100 ML | 5% | 25 | mL |
| (AB) | | | | | |
| Gentamicin 50 mg/mL | Sigma | G1397-10 mL | 10 μg/mL | 105 | μL |

TABLE 4
Reagents for 50/50 Medium

| | Final Concentration | | 100 mL | |
|---|---|---|---|---|
| Complete Medium (CM) | 50% | | 50 | mL |
| AIM-V | 50% | | 50 | mL |
| IL-2 1000 IU/μL | 3000 | IU/mL | 0.3 | mL |
| Anti-CD3 1 mg/mL | 30 | ng/mL | 3 | μL |

In this REP step, 5 million cells of ALI TILs were obtained from the Pre-REP step, and cells were suspended in 100 mL of 50/50 medium. Next, 0.5 million ALI TILs were counted and mixed with 50 million irradiated PBMCs before plating on to each well of G-Rex 6M Well plate. This is day 0 of REP. ALI TILs were added with 3000 IU/mL of IL-2 every 3 days. On day 11 of REP, ALI TILs were collected and filtered through 100 m cell strainer. ALI TILs can be frozen down or used for the experiment/treatment. For comparison, in this REP step, ALI TILs were replaced with STD TILs.

Co-Culturing of ALI TILs or STD TILs with Autologous Tumor Epithelial Cells

ALI TILs and STD TILs were co-culture for 48 hours with autologous tumor epithelial cells derived from submerged Organoids. Single Cell tumor Organoids alone were used as a control. At 48 hours post-incubation, cells were collected for Flow Cytometry analysis.

### Flow Cytometry to Assess Tumor Killing Capacity of ALI TILs and STD TILs

Cells were stained with fluorochrome-conjugated antibodies for CD45, EPCAM, Annexin V, Zombie NIR live dead and other T cell markers. Flow Cytometry analysis for live tumor cell count was performed after staining.

### Results

FIG. 1 depicts the timeline and treatment of pre-Rapid Expansion Protocol (pre-REP) step of ALI TILs and STD TILs. In this present disclosure, the pre-REP of ALI TIL has additional 1-2 weeks of low IL-2 treatment as compared to the pre-REP of STD TIL.

To assess the tumor killing capacity of TILs, the absolute number of tumor cells in the conditions where autologous tumor organoids were co-cultured with TILs was divided by the absolute number of tumor cells in the conditions where autologous tumor organoids were cultured without TILs (control condition). As shown in FIG. 2, co-culturing of ALI Tumor Infiltrating Lymphocytes (ALI TILs) with autologous tumor epithelial cells demonstrated a better tumor killing capacity compared to co-culturing of standard TILs (or STD TILs) with autologous tumor epithelial cells. The result of these analyses was expressed on percentage of live tumor epithelial cells. Data was shown for samples derived from 3 different patients (CRC-1=colorectal cancer patient 1, CRC-2=colorectal cancer patient 2, MLN=melanoma patient). 3 replicates per experiment were performed. Nonparametric ANOVA tested corrected by Geisser Greenhouse Correction was performed to compare between different conditions; *≤Pv=0.05.

### Example 2: ALI Tumor Infiltrating Lymphocytes (TILs) were More Reactive to Tumor Cells Compared to Standard TILs

In this example, reactivity of ALI TILs vs STD TILs was assessed.

PDO organoids were established from primary tumor as described in the previous example.

Co-Culturing of ALI TILs or STD TILs with Autologous Tumor Epithelial Cells

As described in the previous example, ALI TILs and STDTILs were co-cultured for 16 hours with autologous tumor epithelial cells derived from submerged organoids. ALI TILs or STD TILs alone were used as a control. In this experiment, at 16 hours post-incubation, cells were collected for Flow Cytometry analysis to check the expression of IFNγ and CD107a.

### Flow Cytometry to Assess Reactivity of TILs Toward Tumor Cells

Cells were stained with fluorochrome-conjugated antibodies for IFNγ secretion, CD107a, CD45, EPCAM, Annexin V, Zombie NIR live dead and other T cell markers. Flow Cytometry analysis for live CD3+ cells was performed after staining.

### Results

To assess the tumor-reactive of TILs, the percentage of CD3+ cells secreting IFNγ and the % of CD3+ cells expressing CD107a on their surface were assessed. FIG. 3A depicts co-culture experimental timeline. As shown in FIG. 3B-3C, ALI TILs showed higher percentage of IFNγ and CD107a compared to STD TILs, suggesting that ALI TILs were more reactive to tumor cells compared to STD TILs. Data was shown for samples derived from 3 different patients (CRC-1=colorectal cancer patient 1, CRC-2=colorectal cancer patient 2, MLN=melanoma patient). 3 replicates per experiment were performed. Nonparametric ANOVA tested corrected by Geisser Greenhouse Correction was performed to compare between different conditions; *≤Pv=0.05.

### Example 3: ALI Tumor Infiltrating Lymphocytes (TILs) Expressed Higher Levels of HLA-DR Compared to Standard TILs

In this example, the levels of PD1, HLA-DR, and CD137 were assessed.

PDO organoids were established from primary tumor as described in the previous example.

Co-Culturing of ALI TILs or STD TILs with Autologous Tumor Epithelial Cells

ALI TILs and STD TILs were co-cultured with autologous tumor epithelial cells derived from submerged Organoids for 48 hours. ALI TILs or STD TILs alone were used as a control. 48 hours post-incubation, cells were collected for Flow Cytometry analysis.

### Flow Cytometry to Measure Levels of PD1. HLA-DR, and CD137

Cells were stained with fluorochrome-conjugated antibodies for HLA-DR, CD137, PD1, CD45, EPCAM, Annexin V, Zombie NIR live dead and other T cell markers. Flow Cytometry analysis for live CD3+ cells was performed after staining.

### Results

To assess the tumor-reactive of TILs, the MFI (Median Fluorescence Intensity) of PD1, HLA-DR and CD137 was quantified and compared to the MFI of ALI TILs or STD TILs that were not exposed to tumor cells. FIG. 4A depicts an experimental timeline for co-culture experiment. FIG. 4B-4D show flow cytometry result of PD1, HLA-DR, and CD137. As shown in FIG. 4C, in all samples, ALI TILs showed higher level of HLA-DR compared to STD TILs. The result is presented on Fold Change. Further, as shown in FIG. 5, AIL TILs showed higher percentage of HLA-DR+ than STD TILs, at least about 50%, 23%, and 35%, in CRC-1, CRC-2, and Melanoma (MLN), respectively. Data is shown for samples derived from 3 different patients (CRC-1=colorectal cancer patient 1, CRC-2=colorectal cancer patient 2, MLN=melanoma patient). 3 replicates per experiment were performed. Nonparametric ANOVA tested corrected by Geisser Greenhouse Correction was performed to compare between different conditions;*≤Pv=0.05

### Example 4: Air-Liquid Interface Tumor Infiltrating Lymphocytes

Provided here is an example of a method that combined the ALI and TIL processes to selectively re-activate and expand tumor rejection antigen-recognizing T cells, and yielded a TIL product with greater anti-tumor activity than standard TILs. Infiltrating lymphocytes were initially cultured along with their tumor organoid prior to the expansion phase, yielding TIL preparations with expected features relative to standard preparations. Methods were performed as described in the Example 1.

### Successful Implementation of ALI Tumor Organoid Technology

As demonstrated in FIG. 6A and FIG. 6B, the organoids were successfully grown from fresh tumor tissues (e.g., kidney, lung, esophagus, uterus, and Glioblastoma Multiforme (GBM)) using the air-liquid interface (ALI). FIG. 6A shows representative brightfield images of ALI tumor organoids from kidney, lung, esophagus, and uterus cancer. For the samples used to produce ALI TILs, the organoids were eventually maintained under submerged conditions that provide robust stem cell-based cancer models to allow extensive passaging and cryopreservation for subsequent in vitro and in vivo assessments (FIG. 6B). FIG. 6B shows a colorectal cancer (CRC) tissue (left) and a GBM sample (right) grown as ALI organoids for TIL expansion (top) and submerged organoids for assays (bottom). Cancer origin of the CRC organoids was confirmed by whole genome/exome sequencing, which matched the clinical sequencing of the original tumor (FIG. 6C). As shown in FIG. 6C, sequencing results for 3 CRC organoid lines were shown as CNV plots and mutated genes (APC, TP53, KRAS).

### ALI Tumor Organoid-Based Process Produced Clinically Relevant TILs

A research-scale process was developed that integrates an initial ALI tumor organoid and infiltrating lymphocytes co-culture phase, that represents the pre-REP first time period, during which cells were treated with low dose of IL-2, followed by a T cell outgrowth phase in the presence of high dose IL-2 (representing the second time period of the pre-REP step), and a rapid expansion phase in the presence of IL-2, anti-CD3, and irradiated PBMC (during REP step) (FIG. 7A). FIG. 7A shows a schematic representation of the 2-step ALI-TIL process and cryopreservation of the product. This process was successfully performed using different samples including CRCs, gastric cancer, non-small cell lung cancer (NSCLC), renal cell carcinoma (RCC), and melanoma. Cells were counted upon harvest and cryopreserved to mimic product formulation. Extrapolated full-scale yields averaged 11×10⁹ cells (75×10⁷ to 64×10⁹) numbers that fall within the cell dose of 1×10⁹ to 150×10⁹ administered in the clinic (FIG. 7B). As shown in FIG. 7B, ALI-TILs obtained from 14 preps were counted and numbers factored to account for tissue sampling and pre-REP dilution. Estimated full-scale yields are plotted for each individual prep with average and SEM (left). Box plot illustrates the fold-expansion distribution of the 14 REP products (right). To characterize the ALI-TIL product, thawed cells were analyzed by FACS. Consistent with the TIL phenotypes, ALI-TILs were mainly comprised of αβ T cells of the CD4 and CD8 lineages, each made of ˜83% effector memory T cells (TEMs) and ˜16.5% central memory T cells (TCMs). Two other subsets, naïve/TSCMs and effector memory CD45RA+ T cells (TEMRA), were present at <1% on average (FIG. 7C). FIG. 7C shows that twelve ALI-TILs preps (original 14 minus 2 CRCs for which material was insufficient) were analyzed by flow on an Agilent Quanteon analyzer (Santa Clara, CA), using CD3, γδ TCR, CD4, CD8, CD45RA, and CD62L to detect T cells, lineage, and memory subsets. Results were analyzed with FlowJo (Ashland, OR) and plotted as percent parent for each individual sample with average and SEM. This high-level analysis shows that TILs generated with the ALI process present with the expected phenotypic characteristics of a TIL product.

### Establishment of In Vitro Cell-Based Assays for TIL Functional Characterization

Tumor reactivity was tested by co-culturing the ALI-TILs from CRC organoids with autologous tumor cells established in a parallel organoid culture, grown as conventional organoids that exclusively possess tumor cells and not immune cells. As shown in FIG. 8A, increased IFNγ, CD107a, and 4-1BB were all detected by flow cytometry in 0.12 (6.3-fold induction relative to MHC block control), 0.8 (3.8×), and 0.4 (1.6×) % of T cells, respectively. In FIG. 8A, ALI-TILs from 1 CRC were co-cultured with autologous tumor organoids for 16 hours at a 1:1 effector:target ratio. PMA/ionomycin was used as activation control and antibodies generated from clone W6/32 were used to block major histocompatibility complex (MHC) class I (MHC-I) presentation and to confirm antigen specificity. Cells were stained for live/dead, EPCAM, CD3, CD8, and CD107a & IFN-gamma or 4-1BB and analyzed by flow, using a Quanteon instrument (Agilent, Santa Clara, CA). Data were processed with FlowJo (Ashland, OR), gating for indicated marker as shown. This result shows that level of responsive T cells can be used for clinically active TIL products. Further, tumor cell killing was assessed by longitudinal high-content confocal imaging (FIG. 8B). ALI-TILs from 1 CRC were co-cultured with autologous tumor organoids for 24 hours at a 10:1 effector:target ratio. Tumor cell killing was monitored at 0 hour and 24 hours after co-culture using Molecular Devices ImageXpress (San Jose, CA). As shown in FIG. 8B, TILs/autologous organoids co-culture, staining, and detection conditions were established. Reduction in tumor organoids and increase in cell death were observed over time.

### A Pilot Single-Cell RNA Seg Experiment Confirmed ALI-TIL Product Polyclonality and T Cell Subset Diversity

TCR repertoire and gene expression profiles were generated for 4 ALI-TIL preps (3 CRCs and 1 melanoma) using cellular indexing of transcriptomes and epitopes by sequencing (CITE-seq). As shown in FIG. 9A, analyses revealed an average of 2334 unique TCR clonotypes per prep, with individual frequencies following a ‘head and tail’ distribution. Unique paired ap CDR3 sequences (assimilated to TCR clonotypes) were counted and ranked by order of frequencies. Shown in FIG. 9A are # of unique clonotypes relative to # of sequenced cells (upper row) and pie charts of distribution of clonotype frequencies decreasing from red to black with the fraction occupied by the top 20 sum of frequencies (lower row). The cell surface markers data were consistent with the FACS analysis, confirming product purity and T cell subsets. As shown in FIG. 9B, unsupervised clustering of the gene expression data generated 10 T cell subsets, that appear to differentially express differentiation (LEF1, CD27), activation (HLA-DR, CD25), effector (GZMA, PRF1, KLRC1), exhaustion (LAG3, HAVCR2) markers in the CD4 (5 clusters), CD8 (3 clusters), and mixed (2 clusters) lineages. FIG. 9B shows expression levels of 397 immune genes were used to identify 10 clusters using SeqGeq (FlowJo, Ashland, OR), shown as a UMAP with a different color representing each cluster. These preliminary data reveal (1) substantial clonotype diversity in the ex vivo expanded REP product, and that (2) CITE-seq can robustly analyze the proposed GBM rALI-TIL candidate and establish how it may differ from non-reprogrammed, non-ALI TIL preps from the same samples.

### Example 5: Reprogramming ALI-TILs (rALI-TILs)

To generate the necessary amount of T cells, TILs are isolated via mechanical dissociation of tumor tissue and subjected to two in vitro cultivation steps. The steps comprise a pre-rapid expansion protocol (pre-REP) followed by the REP phase. TILs are cultured in the presence of high-dose interleukin-2 (IL-2) to promote expansion, resulting in several fold increase (e.g., at least 10 fold increase) in cell numbers. The expanded TILs are cryopreserved and available for adoptive cell therapy (ACT). A therapeutic product consisting of autologous brain TILs reprogrammed to a stem-like state, called stem memory T cells (TSCMs), are developed for use in ACT against glioblastoma (GBM). To achieve this, immune competent GBM organoids are utilized and induce signaling pathway modifications during the in vitro TIL expansion process to promote differentiation into a TSCM phenotype.

### 1. Identification of Reagents and Culture Conditions to Reprogram TILs to a Stem Cell State

The Interferon-gamma (IFN-γ) signaling pathway is known to inhibit the maintenance and diversity of stem-like T cells, while Notch signaling has been identified as a potent regulator of T cell activation and can convert activated T cells into stem memory T cells (TSCMs), which possess increased self-renewal capacity and are critical for long-term immune memory and effective immunotherapy. By blocking the interferon-gamma (IFN-γ) signaling pathway and activating the Notch signaling pathway during the REP phase of the TIL preparation process, it is hypothesized that a fraction of the terminally differentiated effector memory T cells (TEMs) are reprogrammed into TSCMs, resulting in enhanced anti-tumor activity of TILs.

IFN-γ neutralizing antibodies prevent IFN-γ from binding to its receptors and thereby inhibit downstream signaling pathways while agonistic antibodies targeting Notch receptors induce Notch signal activation. Commercially available monoclonal antibodies (mAbs) can be used to modulate the IFNγ and Notch pathways in TILs.

Cryopreserved pre-REP TILs are used to culture pre-REP TILs in medium supplemented with IFNγ inhibiting or Notch activating antibodies at multiple concentrations to identify the most effective mAb cocktail that can block the IFN-γ signaling pathway and activate the Notch signaling pathway. To validate the successful pathway modulations, the expression levels of Myc, Deltex1, and Hes1 are measured to characterize the Notch activation level, as well as IFNγ-induced genes Gbp5, Irf1, and Ccl2 are measured to assess the level of IFNγ signaling by qPCR 8 hours, 24 hours, and 48 hours post mAb administration.

To optimize the TSCM reprogramming conditions during the REP phase that typically takes 11-14 days, TILs during the REP culture are exposed to the most effective mAb cocktail that simultaneously targets IFNγ inhibition and Notch activation either throughout or for the last 7 days of the REP TIL phase. To enhance TSCM reprogramming conditions potentially further, IL-2 is partially substituted with IL-7 and/or IL-15 in the culture to additionally improve the TIL reprogramming process through IFN-γ inhibition and Notch activation. Each of the conditions on a minimum of 10 pre-REP TIL preparations, comprising melanoma, colon cancer, lung cancer, and GBM pre-REP TIL sample is tested.

To verify TSCM reprogramming, the cultured cells are evaluated for the expression levels of markers, including CCR7, CD45RA, CD62L, CD69, CD95, and CD103, that facilitate the identification of distinct T-cell memory subtypes are assessed. These subtypes are (i) central memory T cells (TCM), which are characterized by CD45RA-CCR7+CD62L+ expression, (ii) tissue resident memory T cells (TRM), which are identified by CD69+CD103+ expression, (iii) effector memory T cells (TEM), which are identified by CD45RA-CCR7-CD62L-expression, (iv) stem cell memory T cells (TSCM), which are characterized by CD45RA+CCR7+CD62L+CD95+ expression, (v) Naïve T cells, which are characterized by CD45RA+CCR7+CD62L+ expression, and (vi) terminally differentiated effector memory cells (TEMRA), which are identified by CD45RO-/CCR7—expression. To characterize T cell function, differentiation, activation and exhaustion, the expression levels of immune checkpoint proteins such as PD-1, Tim-3, LAG-3, TIGIT, and CTLA-4, which are commonly associated with impaired T cell function and increased susceptibility to apoptosis during cancer progression (REF) are assessed. The system yields a detectable TSCM subset of at least 5% and a TCM subset of at least 20%. These subsets are of particular interest because they are associated with enhanced persistence, proliferation, and efficacy in adoptive T cell therapy for cancer. By detecting these subsets, the effectiveness of the reprogramming protocol and identification of areas for further optimization to improve the yield of TSCMs are evaluated.

Alternative approaches to ensure that antibodies do not affect other pathways or have any unintended off-target effects on the cells comprise the use of small-molecule inhibitors that can target specific molecules within the IFN-γ pathways or the administration of exogenous Delta-like1 ligand to activate Notch signaling.

2. Development of an ALI Organoid-Based Process for the Generation of Reprogrammed TILs (rALI-TILs) from at Least 4 Primary GBM Samples

To enhance the therapeutic potential of TILs for GBM, donor- or patient-derived air-liquid interface (ALI) immuno-competent tumor organoids is cultured. By using the ALI culture system, a physiologically relevant microenvironment that better mimics the in vivo tumor niche than traditional cell or tissue culture is established while being less immunosuppressive than the in vivo tumor microenvironment (TME). The ALI-TIL process favors the selection of T cells recognizing tumor neo antigens and leads to a shift of the T cell receptor (TCR) repertoire of TIL preparations towards enhanced neoantigen recognition capabilities. Consequently, these conditions enrich the TIL product for GBM specificity, function and achieve better outcomes in the fight against GBM.

To generate GBM ALI TILs, ALI GBM organoids containing TILs are cultured in GMB medium supplemented with a low dose of IL-2 to allow for the growth of GBM ALI TILs. After 1-7 days GMB ALI organoids are isolated by digesting the collagen block with Collagenase IV. Isolated organoids containing TILs are transferred into a 24-well G-Rex culture dish, followed by a pre-REP cultivation process. During this 11-14 day process, TILs are cultured in T cell medium enriched with high-dose IL-2. After 11-14 days ALI pre-REP TILs are transferred into a 6M G-Rex well to initiate the REP phase. This protocol involves stimulating TILs with anti-CD3 antibody, along with high-dose IL-2, in vitro for 11-14 days. The ALI-TIL process for GBM through two successful runs can each yield a minimum of 250 million cells. rALI-TILs (reprogrammed ALI-TILs) are expected to exhibit a T cell-dominant phenotype, with a minimum of 90% T cells represented by both CD4 and CD8 lineages. This high degree of T cell representation is essential for the effective targeting of cancer cell.

To identify the most effective conditions for enhancing the percentage of TSCM in the GBM ALI-TIL preparations, the composition and concentration of the supplemented mAbs and other growth factors including IL-2, IL-7 and IL-15 are further optimized. This approach helps direct GBM TILs towards an immunophenotype, which is expected to enhance their anti-tumor persistence. In order to investigate the efficacy of optimized stem cell reprogramming protocols for GBM rALI-TILs, rALI-TILs are from at least 4 different GBM samples and their T cell memory phenotype is compared to the matched control TIL preparations, including ALI, CTRL, and CTRL TILs. After TIL preparations have been generated, yields are determined. From this, the full-scale TIL yields is extrapolated, which provides insights of the potential for the TIL generation processes in clinical applications. To ensure the long-term preservation of the samples, the TIL preparations at 20 million cells per vial are aliquoted and cryopreserved. This allows assessing the samples at a later time, if needed, and to continue the investigations into the efficacy of the optimized reprogramming protocol for GBM rALI-TILs.

A detailed immune-profiling analysis of autologous TIL preparations including ALI, rALIs, autologous control (CTRL), and rCTRL from 4 GBM samples is conducted.

Flow cytometry is used to comprehensively analyze individual the TIL preparations by assessing T cell subtypes, T cell memory and function. The panel of markers comprise CCR7, CD45RA, CD62L, CD69, CD95, CD103, PD-1, Tim-3, LAG-3, TIGIT, and CTLA-4, which is used to differentiate between T cell memory subsets, activation status, and functional potential, as well as to evaluate T cell exhaustion levels. This analysis provides a comprehensive immune profile of GBM ALI-TILs and their autologous controls and identify differences in T-cell subsets and activation status. The selection of the rALI-TIL process is based on a yield of at least 1×10⁸ cells, with a minimum of 90% T cells, and a minimum of 20% TCMs and 5% TSCMs indicating successful reprogramming. To further ensure the purity of the TIL preparation, a flow cytometry panel is established to characterize the expression levels of several cell surface markers linked to glioblastoma, including CD133, CD44, CD45, and CD90. The goal is to identify cell surface markers that are uniquely expressed in autologous GBM cells but absent in their respective TIL preps.

To evaluate the T cell fitness of the TIL preparations, an IFNγ release assay is conducted on all 16 GBM TIL samples. This assay is a widely used method to assess T cell activation and cytokine production in response to pan TCR stimulation. The assay is performed by incubating individual TIL preparations with a pan TCR stimulant consisting of anti-CD3 and anti-CD28 antibodies. Following an incubation period of 1 to 3 days, the supernatant is collected, and the amount of IFNγ released by the cells is measured using ELISA. Utilizing this approach allows evaluation of the TIL samples' responsiveness to pan TCR stimulation and to assess their overall T cell fitness. The secretion of over 200 pg/ml IFNγ serves as an indicator of a robust T cell fitness level.

The co-culture of GMB TILs and submerged Glioblastoma organoids (GBOs) serves as an essential tool to investigate the cytotoxic potential and cytokine secretion profile of TILs against autologous tumor cells in various in vitro assays described below. GBOs are three-dimensional (3D) cultures that mimic the complex architecture and cellular heterogeneity of in vivo GBMs. A GBO model is established by culturing 1 mm³ patient derived GBO tissue fragments in suspension. The organoids are maintained in a serum-free medium and can be propagated for several months. To avoid necrotic cell death in the inner core, GBOs are propagated by cutting them into 0.5 mm diameter pieces every 1-2 weeks. These organoids preserve the genomic and transcriptomic features of the original tumors and can be used to evaluate drug responses and gene expression changes. The goal is to establish and expand this GBO culture system from fresh glioblastoma tissues and cryopreserve them for future experiments. This ensures a continuous supply of GBOs that can be used to characterize the anti-tumor response of matched TIL preparations. To ensure the establishment of high-quality GBO lines, specific criteria is set. A GBO line is established if it can be passaged indefinitely, can be cryopreserved for future use, and exhibits antigen presentation in response to IFNγ exposure. Furthermore, the genotype of the established GBO line is matched to that of the original GBM sample by performing whole-genome sequencing. Meeting these criteria ensures the establishment of reliable and consistent GBO lines for future experiments.

### 3. Comparison of In Vitro Functionality of GBM Reprogrammed ALI TILs Versus Non-Reprogrammed and Control TILs

Well established in vitro assays are employed to characterize and compare the anti-tumor activity of rALI TIL preparation to autologous controls. These assays are used to evaluate the cytotoxicity and cytokine secretion profile of TILs against autologous patient-derived glioblastoma organoids in co-culture experiment. The results of these assays provide a comprehensive understanding of the functional differences between different TIL preparations and help to identify the optimal culture method for generating TILs with enhanced anti-tumor activity.

All GBM samples are expanded for functional TIL testing assays specified below.

To identify the most suitable GBO/TIL co-culture conditions that do not compromise cell viability and function, the focus is on finding a media composition and co-culture system that is equally well-tolerated by both GBOs and TILs. By achieving this, in vitro functional TIL testing assays such as tumor reactivity assays (TRA) and GBO killing assays can be carried out to assess the anti-tumor potential of all 16 GBM TIL preparations.

To evaluate the reactivity of the various GBM TIL preparations against tumor cells, the TRA, an in vitro assay where different TIL preps are co-cultured with patient matched GBOs is used. The production of IFN-γ, CD107a, and 4-1BB is used as a direct read-out of tumor antigen-specific T cell activation and a strong predictor of the T cell's cytotoxicity. IFN-γ is a cytokine produced by activated T cells and is an important marker of T cell function. The presence of IFN-γ in this assay indicates T cell activation and production of this cytokine in response to tumor antigens. CD107a, is a lysosomal-associated membrane protein that is expressed on the surface of activated T cells. The presence of CD107a on the cell surface indicates T cell degranulation and release of cytotoxic molecules, such as perform and granzyme B, that are responsible for killing tumor cells. 4-1BB, is a co-stimulatory molecule expressed on the surface of activated T cells. CD137 signaling enhances T cell proliferation and survival and promotes the production of cytokines such as IFN-γ. The expression of CD137 on T cells in the TRA is used as an additional marker of T cell activation and function. In this experiment, TILs and GBO-derived tumor cells are co-cultured in a 1:1 ratio for a period of 4-24 hours. To ensure the assay's functionality, a PMA/Ionomycin T-cell stimulation cocktail is used as a positive control. To ensure the specificity of the assay, negative controls are used, including a supplement of MHC blocking antibody cocktail, which can block the recognition of tumor antigens by T cells, thereby preventing T cell activation. Additionally, TILs cultured in the absence of corresponding tumor cells also serve as negative controls. After co-culture, all samples undergo flow cytometric analysis to evaluate the expression levels of IFN-γ, CD107a, and CD137 on T cells. This analysis provides a quantitative assessment of T cell reactivity against tumor cells and aid in identifying any variations regarding the tumor reactivity between GBM TIL preparations that are generated via different methods.

To evaluate the tumor cell killing capacity of all GBM TIL samples, GMB TIL/GBO co-cultures at a 10:1 ratio for a 24 h, 48h and 72h are set. Negative controls, including MHC blocking antibodies, are used to ensure assay specificity. After co-culture, the level of TIL-mediated tumor cell killing is evaluated through flow cytometric analysis. This analysis involves the measurement of various parameters, including the expression levels CD3 and GBO specific surface markers, which are used to label T cells and GBM tumor cells, respectively. In addition, the levels of apoptosis and necrosis in tumor cells are measured using Dapi, Annexin V or propidium iodide staining, enabling discrimination of live, apoptotic, and necrotic cells.

To validate the tumor cell killing data obtained from the flow cytometry analysis, live cell confocal imaging is performed to monitor TIL-mediated GBO cell death over a period of 72h. The TILs and GBOs are co-cultured at a 10:1 ratio, and the GBM TILs are labeled with Cell Trace Violet while the GBOs are labeled with Cell Tracker Orange. To visualize cell death in the orange labeled GBOs, Sytox Green is added to the co-culture medium, which is a nucleic acid stain that is impermeable to the cell membrane of living cells but enters dead cells and binds to nucleic acids, resulting in green fluorescence labeling of the dead cells. Negative controls, including MHC blocking antibodies, are used to ensure assay specificity.

A robust correlation between flow cytometry data and live cell imaging results in evaluating TIL-mediated GBO cell death is expected. This proposed approach enables a comprehensive analysis of the cytotoxic potential of TILs against autologous GBM tumor cells and identifies the TIL generation process that is most effective for GBM immunotherapy.

### 4. Testing of the Efficacy of at Least 2 Reprogrammed ALI-TILs in Autologous GBM Organoid-Derived Xenograft (ODX) Mouse Models and Preliminary Mechanism of Action (MOA) Studies.

The efficacy of rALI-TILs in controlling tumor growth in vivo is evaluated by monitoring their ability to control the growth of autologous ODX tumors. To achieve this, organoid platform is used to generate autologous tumor xenograft models. In addition, in vivo persistence is characterized by detecting transferred T cells in the tumor deposits and the circulation over time.

To investigate the tumor-forming potential and growth kinetics, a minimum of four donor-derived GBO lines using a SQ xenograft model in immunocompromised NOG mice is used. The GBOs are dissociated into single cells and small cell clumps, then resuspended in a mixture of 50% GBO medium and 50% Matrigel before transplantation. Each GBO line is implanted in five mice, with doses varying from 1×10⁵ to 1×10⁶, and tumor growth is monitored by caliper measurement weekly until the tumor reaches a size of 2000 mm³, at which point the mice is sacrificed. The tumor tissue is isolated from the mice, banked, and subjected to histological and molecular characterization. The objective of this analysis is to validate that the genotype and phenotype of the ODX tumor tissue are similar to that of the corresponding primary GBM, thereby providing a reliable model for subsequent experiments. In addition to the SQ xenograft model, orthotopic GBM xenograft models is established, which are more clinically relevant than subcutaneous models as they more closely replicate the tumor microenvironment of the brain. These models are used to study tumor invasion and better understand the behavior of GBM in its native environment. To establish the orthotopic GBO xenograft model, GBO-derived tumor cells are injected directly into the brain of immunocompromised mice using a stereotactic injection system through a small hole in the skull to ensure precise targeting of the injection site. Like for the subcutaneous implantation, GBO tumor cells are resuspended in a mixture of 50% GBO medium and 50% Matrigel prior to transplantation. Following implantation, the mice are monitored for neurological symptoms, and tumor growth is measured using MRI. To minimize variability, the GBO dissociation protocol and transplantation methods is standardized. This involves using optimized digestive enzyme concentration and digestion time for each GBO line and employing a stereotactic injection system for orthotopic transplantations to minimize the risk of damage to brain tissue.

To evaluate the cytotoxic anti-tumor activity of GBM rALI TILs in vivo, the ideal xenograft model is employed and utilizes the optimal GBO cell number for tumor formation and growth kinetics for each GBO line. To conduct this set of experiments, immunodeficient NOG mice from Taconic that express human IL-2 at a range of 0.5-2.0 ng/mL of blood is used. Human IL-2 expression in the mouse model is crucial to support survival and function of infused human TILs. At a tumor size of 150 mm³, 36 female mice per GBO ODX line are randomized into three groups. The first and second group receives infusions of 20 million GMB patient-matched rALI TILs and CTRL TILs, respectively, via the tail vein. The third group serves as control. Tumor size is monitored weekly until one of the group reaches a tumor size of 2000 mm³, at which point the mice is sacrificed. At the termination of this GBO tumor xenograft experiment, all remaining tumors from the three groups are harvested and evaluated. Based on the hypothesis that rALI TILs present significantly enhanced cytotoxic anti-tumor activity, the administration of these cells elicits a substantial reduction in tumor volume relative to the control cohorts.

To investigate the in vivo persistence and tumor infiltration of the transferred cells, TIL invasion within the tumor microenvironment and T cell persistence in mouse blood samples is evaluated. The blood samples at various time points after TIL infusion is collected and analyzed using flow cytometry to determine the percentage of human T cells in the mouse blood. This data allows assessment of the in vivo persistence of GMB rALI TILs and autologous controls. Then, tumor samples at the termination of the GBO tumor xenograft experiment are collected and flow cytometry is performed and immunohistochemical staining to assess the extent of TIL infiltration within the tumor microenvironment. This provides crucial information on the localization of the TILs within the tumor and their potential impact on tumor growth.

To validate the tumor antigen-specific cytotoxic killing-mediated MOA of rALI-TILs, anti-tumor activity against allogeneic ODX models is assessed and the effect of CD8+ cell depletion is investigated in the TIL preparation prior to infusion. Firstly, non-matched rALI-TILs are infused into mice harboring ODX tumors of 150 mm³ in size and tumor growth is monitored using the methodology described above. This determines whether rALI-TILs exhibit non-specific or antigen-specific cytotoxic killing against ODX tumors. Secondly, autologous rALI-TILs are infused into mice with ODX tumors, with one group receiving a cytotoxic CD8+ T cell depleted TIL product. A significantly decreased anti-tumor activity in the CD8+ depleted group is expected, validating neoantigen-specific CD8+ cell derived cytotoxic tumor cell killing in the in vivo experiments.

### 5. Characterization of TCR Repertoire and Gene Expression Profile of Reprogrammed Versus Non-Reprogrammed TILs at the Single Cell Level.

The T cell receptor (TCR) repertoire of a TIL preparation represents the diversity and composition of TCRs expressed by T cells within the sample. The TCRs expressed by each T cell recognize a specific antigen presented by major histocompatibility complex (MHC) molecules, thereby reflecting the specificity and diversity of the T cell response. To confirm that the ALI-TIL process might favor the selection of T cells recognizing tumor neo antigens and lead to a shift of the TCR repertoire within, GBM-derived TIL TCR repertoire is analyzed.

To evaluate the impact of different ex vivo TIL generation and expansion processes on the TCR clonal diversity, TCR sequencing (TCRseq) on 1×10⁴ single cells per GBM derived TIL preparation is performed. A minimum of 4 GBM TIL sets, including autologous CTRL TIL, rCTRL TIL (reprogrammed CTRL TIL), ALI TIL and rALI TIL preparations per GBM samples are analyzed. A distinct TCR clonotype composition between ALI and CTRL autologous samples may suggest that the ALI-TIL process selects for T cells recognizing tumor antigens. Altered clonotype frequencies in reprogrammed TILs compared to non-reprogrammed TILs may reflect the effect of pathway modulations during TIL reprogramming. By understanding these differences, TIL production and selection methods for effective GBM immunotherapy can be further optimized. Gene expression profiling of all GBM-derived TIL preparations at the single-cell level using single-cell RNA sequencing (scRNA-seq) and CITE-seq allows a thorough validation of the proposed reprogrammed stem cell-like state in TILs, exposed to pathway modulations.

Sequencing data analyses using commercial (SeqGeq), academic (GLIPH258), and in-house (R script) pipelines are used to analyze the sequencing data. A commercial pipeline called SeqGeq is used to perform a comprehensive analysis of the sequencing data. This pipeline is useful for the characterization of TCR clonotypes and the RNAseq derived immune profiling data to validate the upregulation of TSCM and TCM markers and downregulation of exhaustion markers in reprogrammed TILs compared to the control group. Secondly, an academic pipeline called GLIPH258 is used to identify TCR clonotypes based on sequence homology. This pipeline is useful for identifying clonotypes that share common features and may recognize similar antigens. Lastly, an in-house R script pipeline is used to perform customized in-depth analyses of the sequencing data. This pipeline is instrumental to determine the TCR clonotype overlap and diversity as well as altered clonotype frequencies between autologous TIL groups.

### Example 6: Tumor Organoid-Derived tumor-infiltrating lymphocyte (TIL) Therapy for Colorectal Cancer

In this example, tumor organoid-derived TIL were generated using methods described herein. Experiments were performed to assess TILs.

### BACKGROUND

Colorectal cancer (CRC) represents ˜7% of new cancer cases and 11% of cancer deaths worldwide. Tumor-Infiltrating Lymphocyte (TIL) immunotherapy is efficacious against melanoma, but its potency in epithelial cancers such as CRC remains inconsistent. This inconsistency is likely attributed to the unselective ex vivo expansion of TILs, which includes nonspecific bystander T-cell clone expansion.

The air-liquid interface (ALI) tumor organoid system described herein allows direct air exposure, supporting the survival of diverse cell types, including the full repertoire of immune cells within the tumor microenvironment. This culture system provides the first accurate and functional model of interactions between tumor cells and immune cells in individual patient samples.

The methods and compositions described herein pioneer an advanced TIL generation method, leveraging ALI technology to overcome the challenge of low-frequency tumor-specific T cells. The methods described herein enhance the efficacy of TIL therapy, particularly for non-melanoma solid tumors like CRC.

### Method

ALI CRC organoids were generated by embedding intact tumor fragments within a collagen matrix on top of a permeable support membrane exposed to both air and culture medium facilitating optimal oxygenation as described in the present disclosure.

FIG. 1 depicts the timeline of pre-Rapid Expansion Protocol (pre-REP) in Standard Tumor Infiltrating Lymphocytes (STD TIL) protocol and in air-liquid interface Tumor Infiltrating Lymphocytes (ALI TIL) protocol. Standard (“STD”) refers to immune cells obtained from a method involving obtaining and culturing TILs from fresh tumor tissue and treated with one concentration of cytokine during the pre-REP step. Briefly, on Day 0 of the STD pre-REP step, the tumor tissue fragments are transferred to G-Rex10 plate. Each well is added with 5-30 tumor tissue fragments in 10 to 40 mL of culture medium comprising RPMI, 10% FBS, gentamicin, and 6000 IU/mL of IL-2. The tumor tissue fragments are then cultured at 37° C. in 5% CO2. On Day 5 and Day 10 of the STD pre-REP step, half medium is removed and replaced with fresh medium and 6000 IU/mL of IL-2. After Day 10, half medium is changed every 2 to 3 days. At the end of day 11, cells are collected before being treated with cocktails for Rapid Expansion Protocol (REP) treatment. Certain types of immune cells are sorted from the tumor tissue fragments for REP step.

In contrast to STD TIL protocol, as described in this present disclosure, in ALI TIL protocol, the starting samples are patient derived organoids (PDOs), which are generated from tissue samples. These PDOs are cultured in air-liquid interface (ALI) environment. These PDOs comprise various immune populations, e.g., tumor infiltrating immune cells, lymphocytes, T cells, etc., which can then be used for expansion. In the pre-REP phase of ALI TIL protocol, the PDOs or ALI organoids are treated with 50 IU/mL of IL-2 for 1-2 weeks before being treated with higher concentrations of IL-2 (6000 IU/mL) for 11 days. At the end of this, cells are harvested for REP treatment.

To characterize ALI TILs following ex vivo expansion, flow cytometry was employed to analyze cell surface markers. Simultaneously, single-cell RNA sequencing was used to examine the immune gene expression profiles. Autologous submerged organoids were established and characterized to assess the in vitro tumor killing capacity of patient-matched ALI TILs. Additionally, organoids were transplanted into mice as organoid-derived xenografts (ODXs) for the evaluation of ALI TILs antitumor activity in vivo. T cell cytotoxicity was measured as tumor cell death upon in vitro co-culture, while preclinical in vivo efficacy was assessed as tumor growth inhibition.

### Results

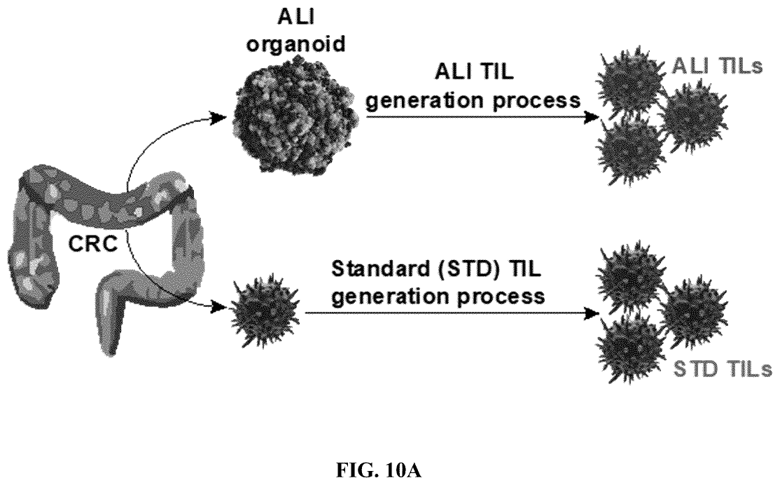

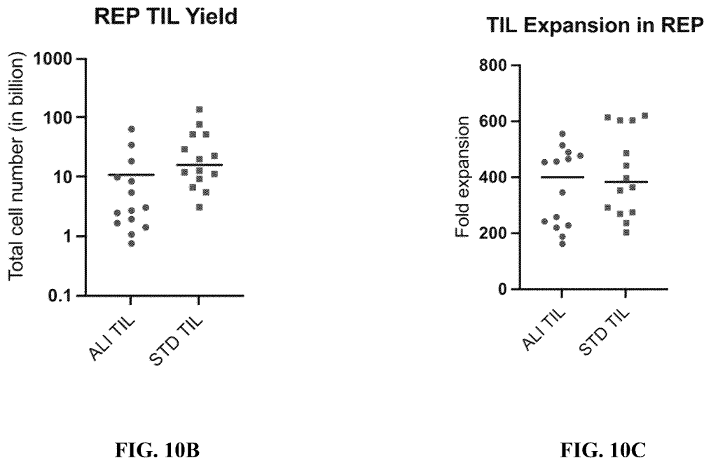

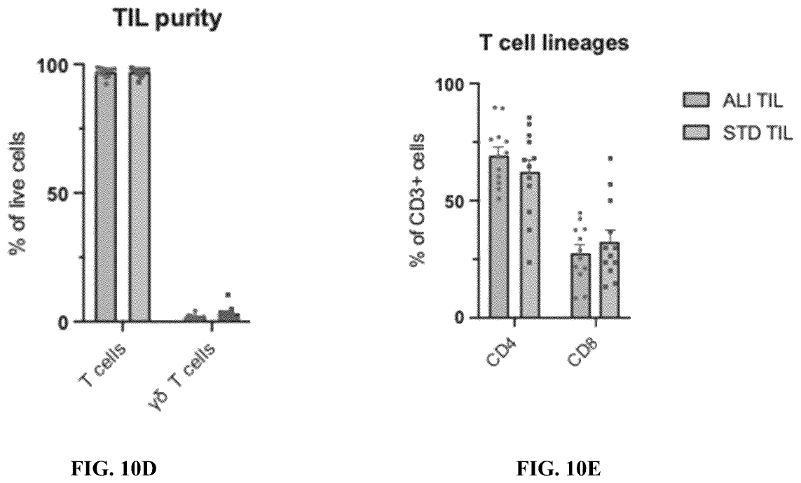

FIGS. 10A-10E demonstrate that the ALI-TIL protocol as described herein yields clinically-relevant numbers of T cells. FIG. 10A shows a schematic of the method in which colorectal cancer (CRC) samples were processed in parallel as ALI (purple) and standard (STD, green) TILs. Full-scale REP yields were estimated by counting TILs from 14 independent donors, accounting for tissue sampling and pre-REP dilution. The ALI TIL process yielded an average of 10 billion cells per prep, and this number provided clinically relevant cell numbers (FIG. 10B). FIG. 10C illustrates the distribution of fold-expansion during the REP process for the 14 CRC TIL preparations. FIG. 10D shows TIL purity, which was assessed using flow cytometry on 12 CRC TIL pairs with CD3 and γδTCR antibodies. FIG. 10E shows CD4+ and CD8+ T cell lineages, which were detected by flow cytometry. FlowJo (Ashland, OR) was employed for analysis, and the results are presented as a percentage of the parent for each sample, with averages and standard error of the mean.

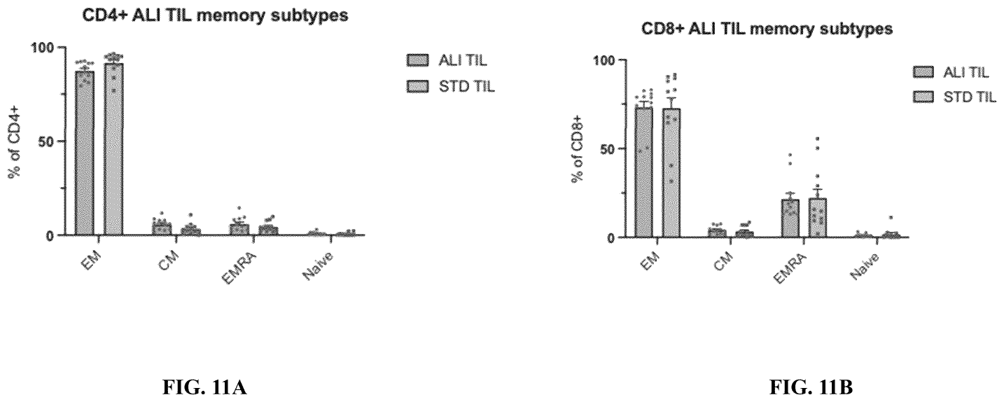

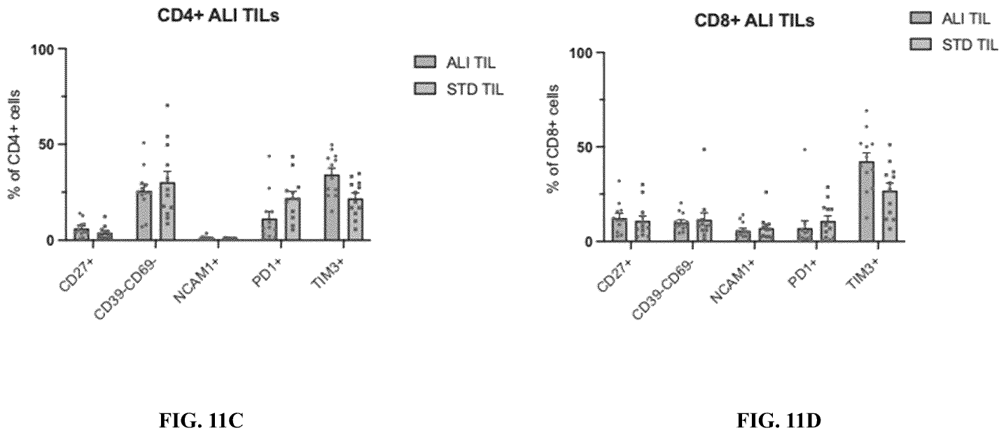

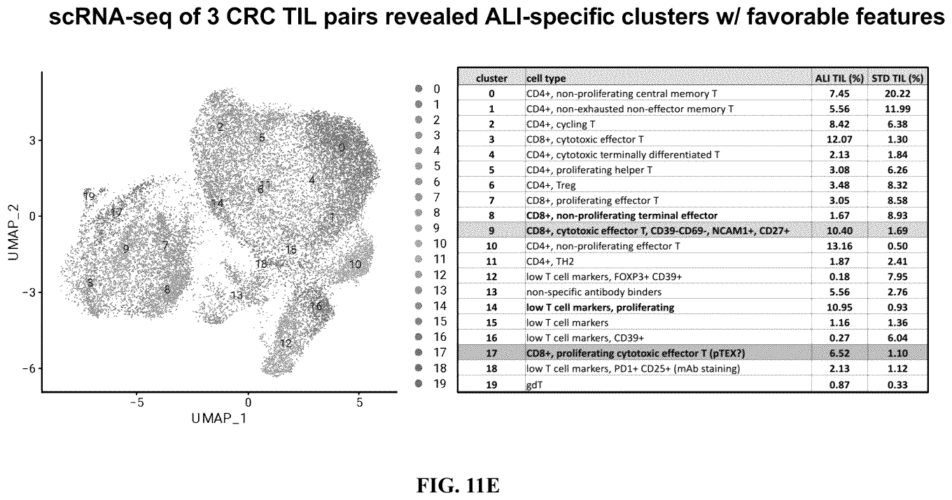

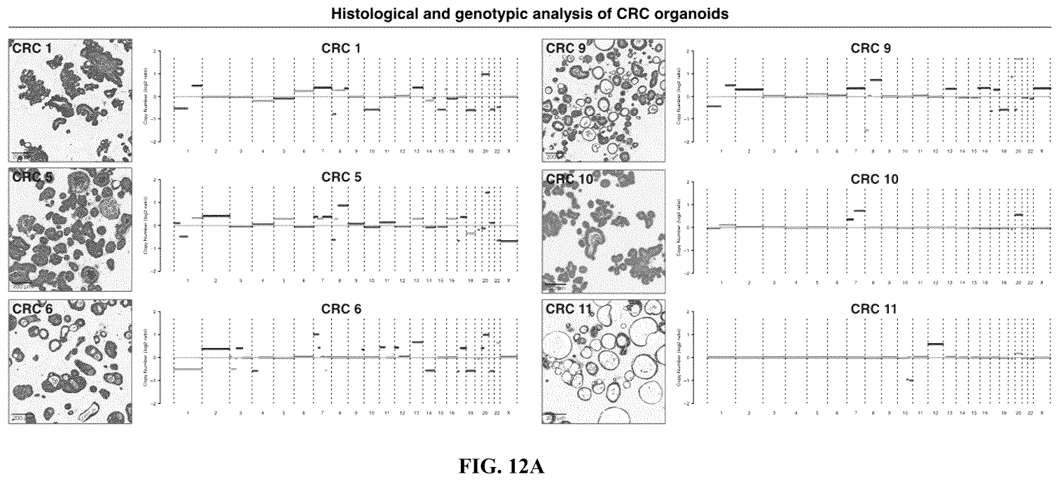

FIGS. 11A-11E show phenotypic characterization of ALI TILs, generated according to the methods described herein. FIGS. 11A and 11B show T cell memory subsets including effector memory (EM), central memory (CM), EM CD45RA+(EMRA), and naïve, which were identified by evaluating CD45RA and CCR7 differential expression on CD4+(FIG. 11A) and CD8+(FIG. 11B) cells. FIGS. 11C and 11D show CD27, CD39, CD69, NCAM1, PD1, and TIM3, which were used as markers of differentiation/stemness and activation/exhaustion, respectively. FlowJo (Ashland, OR) was employed for analysis, and the results are presented as a percentage of the parent for each sample, with averages and standard error of the mean. FIG. 11E shows expression levels of 397 immune genes which were obtained by scRNA-seq and used to identify 19 clusters with SeqGeq (FlowJo, Ashland, OR). Results are shown as a UMAP with a different color representing each cluster. Cell types corresponding to each cluster were determined based on published reports.

Based on the phenotypic characterization of ALI TIL on expected T cell memory subsets and state, the effector memory (EM) subset represented ˜80% of T cells and expression of activation/exhaustion markers was characteristic of recently expanded and reinvigorated T cells.

Results from scRNA-seq also show favorable antitumor biomarkers that ALI TILs were enriched for double negative CD39/CD69 and progenitor exhausted CD8+ T cell clusters.

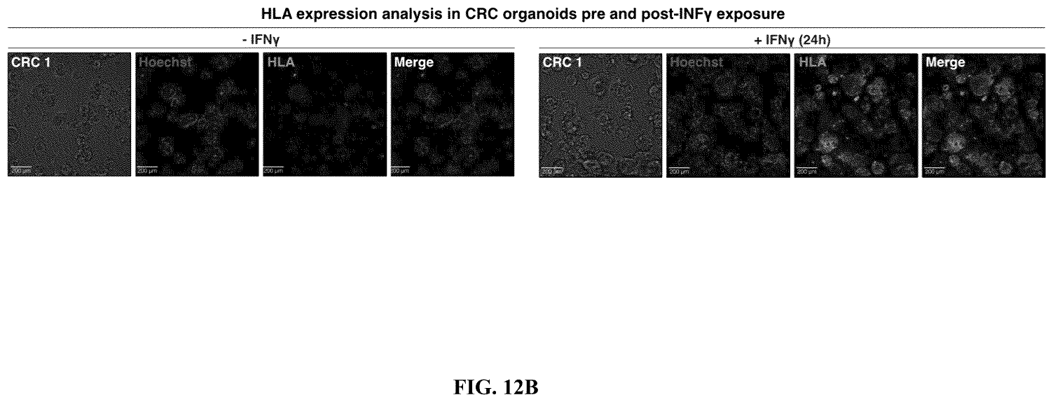

FIGS. 12A and 12B show CRC submerged organoid characterization. FIG. 12A shows representative H&E-stained sections, which unveil diverse morphologies of submerged epithelial tumor organoid lines derived from six CRC donor samples. The corresponding genome-wide analysis maps chromosome copy number variations (CNVs) are shown on the left. The color spectrum denotes CNVs, with dark green (1 copy), blue (2 copies), brown (3 copies), red (4 or more copies), and a light green line indicating the presence of subclone fractions. FIG. 12B shows immunofluorescence confocal microscopy images of HLA-stained sections from CRC1 submerged epithelial tumor organoids, pre-(left) and post-(right) INFy exposure. HLA expression of remaining CRC organoid lines was verified by flow cytometry (data not shown) to confirm antigen presentation competency across all lines.

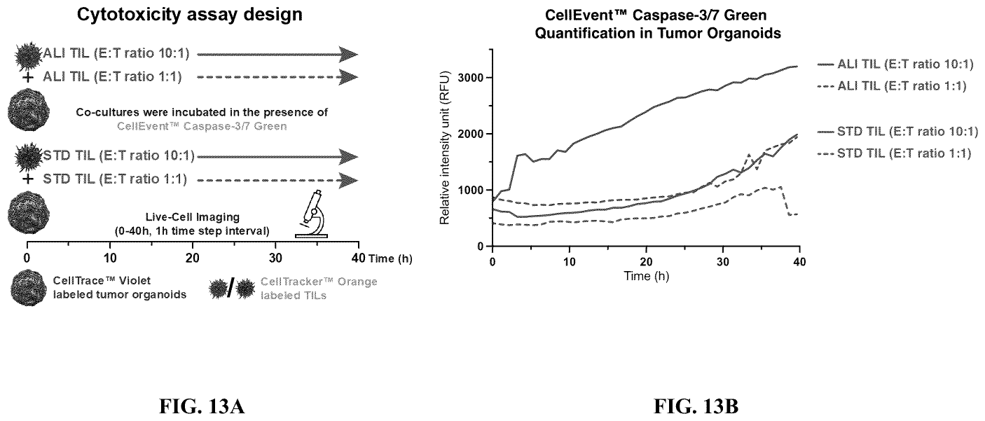

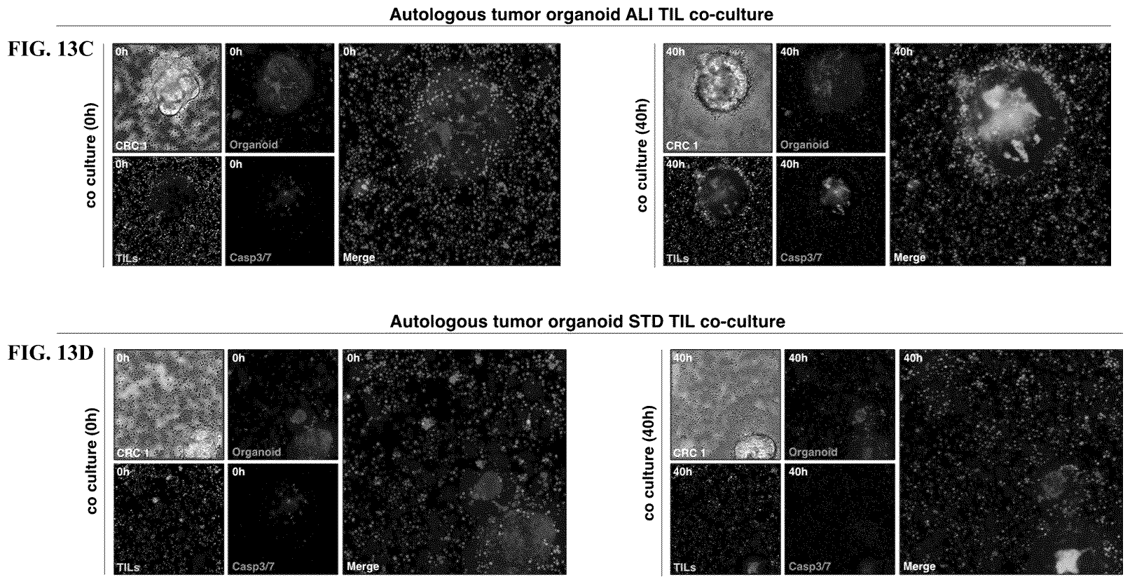

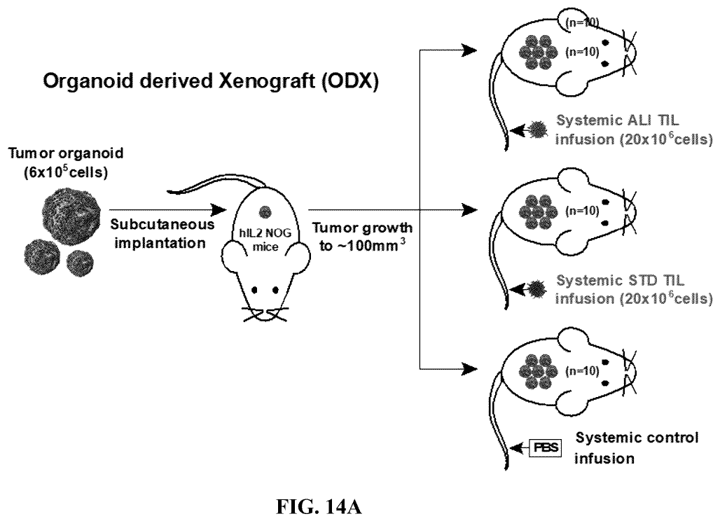

FIGS. 13A-13D show detection of ALI-TIL cytotoxicity in CRC organoids. FIG. 13A shows schematic representation of the ALI TIL cytotoxicity assay. ALI and STD TILs from CRC1 were co-cultured with autologous submerged tumor organoids for 40 hours at 10:1 and 1:1 effector-to-target ratios. Co-cultures were incubated in the presence of CellEvent™ Caspase-3/7 Green Detection Reagent to monitor cell death and analyzed via a Molecular Devices ImageXpress system (San Jose, CA). FIG. 13B shows graph illustrating caspase-3/7 activation in tumor organoids over time in ALI TIL and STD TIL co-cultures. Solid lines represent 10:1, dashed lines represent 1:1 ratios. Notably, tumor organoids co-cultured with ALI TILs display increased levels of caspase-3/7 activation relative to those co-cultured with STD TILs, indicative of greater ALI TIL cytotoxicity. FIG. 13C shows representative confocal images at Oh (left) and 40 hours (right) showing submerged tumor organoids co-cultured with ALI TILs. Tumor organoids labeled with CellTrace™ Violet, TILs with CellTracker™ Orange, and apoptotic cells emitting green fluorescence due to CellEvent™ Caspase-3/7 activation. FIG. 13D shows images at 0 hours and 40 hours illustrating submerged tumor organoids co-cultured with STD TILs. Similar labeling and apoptotic cell fluorescence as seen in FIG. 13C. Based on these result, ALI TIL shows antitumor activity that ALI TILs exhibited specific in vitro tumor cell killing (high cytotoxic activity).

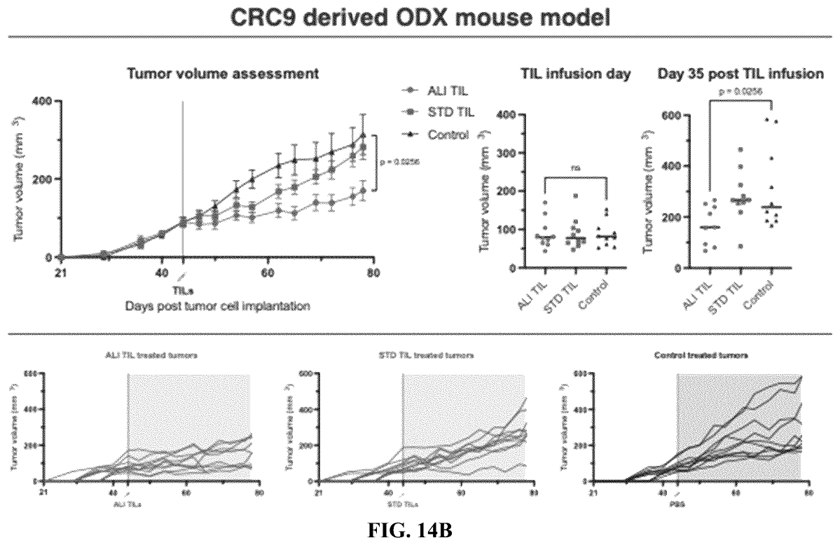

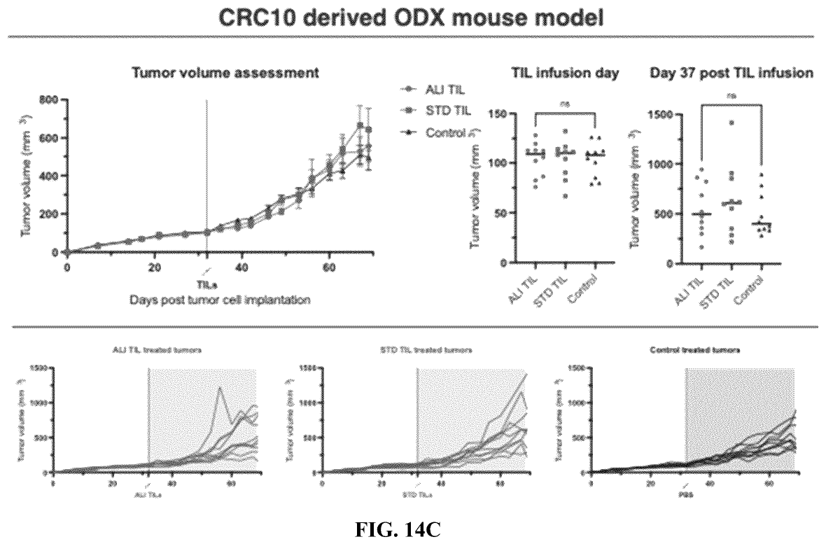

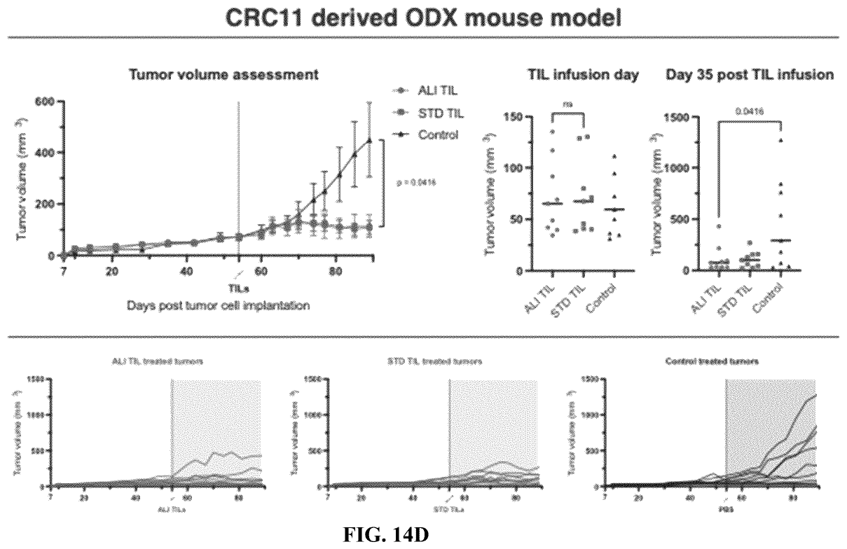

FIGS. 14A-14D show assessment of ALI-TIL in vivo efficacy. FIG. 14A shows experimental design: autologous CRC ODX cells are implanted subcutaneously in the flank of humanized IL2 transgenic NOG mice. When average tumor size reaches ˜100 mm³, mice are randomized in 3 groups (n=10) and treated with ALI TILs, STD TILs, and PBS via a single tail vein injection. Tumor volumes are measured weekly by caliper. FIG. 14B shows tumor growth curves recorded for CRC9. FIG. 14C shows tumor growth curves recorded for CRC10. FIG. 14D shows tumor growth curves recorded for CRC11.

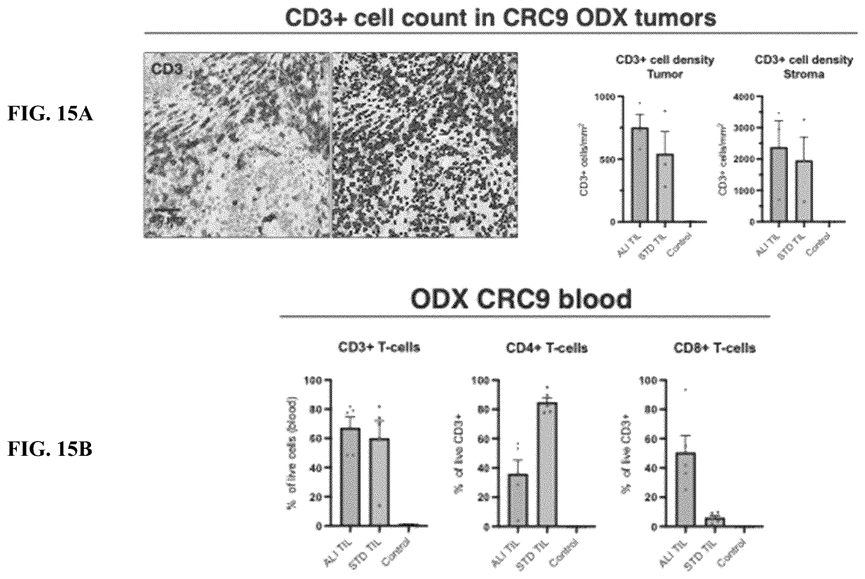

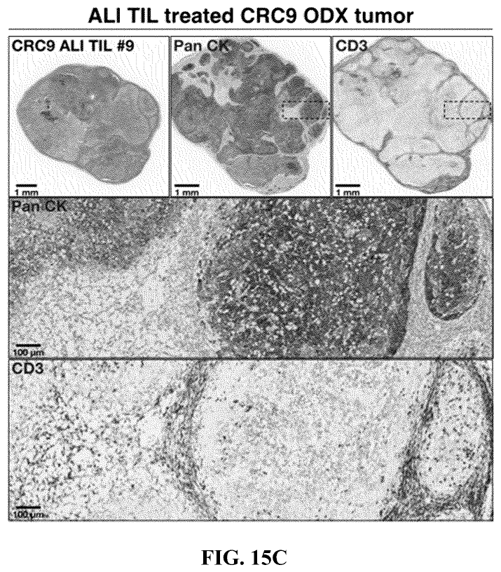

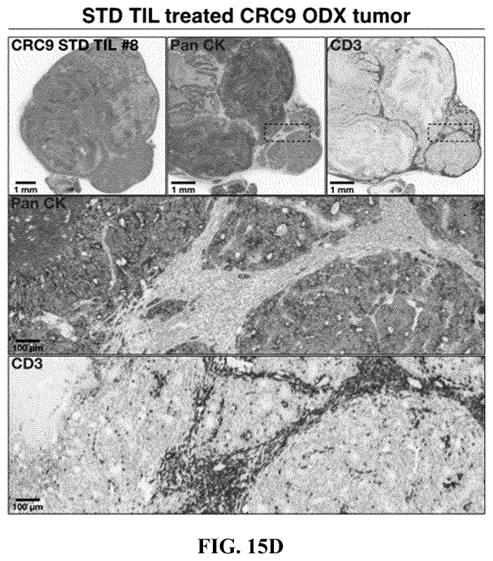

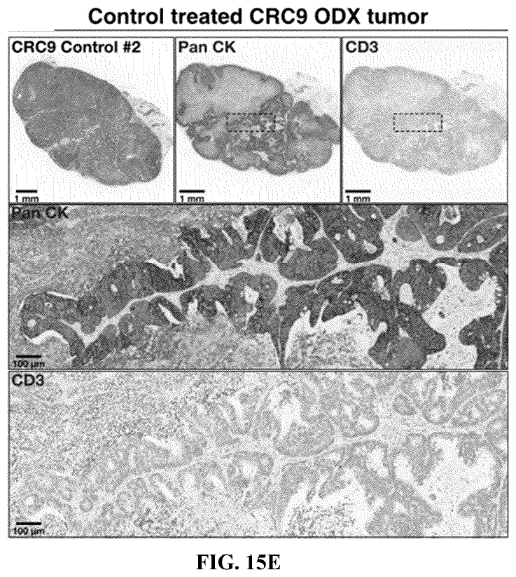

FIGS. 15A-15E show assessment of ALI-TIL in vivo distribution. TILs and vehicle-treated CRC9 tumors were harvested 35 days post-infusion and stained by immunohistochemistry for pan CK and CD3 (FIG. 15A). Number of CD3+ T cells detected in the tumor tissue and in the tumor stroma was counted for 3 samples of each treatment group using an image analysis software. Results are plotted as mean density+SEM in tumor (left) and stroma (right) for ALI TIL (purple bars) and STD TIL (green bars). Circulating TIL-derived T cells were quantified by flow cytometry for 5 mice of each group at Day 35 and represented as in before (FIG. 15B). FIGS. 15C-15E are representative images of H&E, pan CK, and CD3 stained tumors are shown for each of the ALI TIL (FIG. 15C), STD TIL (FIG. 15D), and control (FIG. 15E)—treated group.

ALI TILs demonstrated in vivo antitumor activity against autologous organoid derived xenograft (ODX) models. These results show effective tumor growth inhibition.

Overall, these data present an innovative TIL immunotherapy approach for CRC. The implementation of ALI organoid culture conditions prior to ex vivo TIL expansion yielded a cell product showcasing not only appropriate but compelling phenotypic and functional attributes.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of generating subject derived immune cells, the method comprising:
(a) obtaining one or more tumor samples from a subject, wherein the one or more tumor samples comprise immune cells;
(b) incubating the one or more tumor samples using an in vitro culture process, wherein the in vitro culture process comprises an air-liquid interface setup, wherein at least one cytokine is added to the culture medium; and
(c) collecting the immune cells from the one or more tumor samples, thereby generating the subject derived immune cells.

2. The method of claim 1, wherein the incubating of (b) comprises a first time period and a second time period, wherein the cytokine is added to the culture media at a first concentration during the first time period and the cytokine is added to the culture media at a second concentration during the second time period, and wherein the first concentration is lower than the second concentration.

3. The method of claim 2, wherein the first time period is at least about 1 day, or about 7 days to about 14 days.

4. (canceled)

5. The method of claim 2, wherein the second time period is at least about 1 day, or about 11 days.

6-11. (canceled)

12. The method of claim 1, further comprising (d) expanding the immune cells obtained from (c) using one or more agents for a time period.

13. (canceled)

14. (canceled)

15. The method of claim 1, wherein the immune cells comprise tumor infiltrating lymphocytes.

16. The method of claim 15, wherein the tumor infiltrating lymphocytes comprise T cells.

17. The method of claim 16, wherein the T cells are selected from the group consisting of: activated T cells, naïve CD8+ T cells, cytotoxic CD8+ T cells, naïve CD4+ T cells, helper T cells, memory T cells, T stem cell memory cells (TSCM), effector memory T cells, NKT cells, γd T cells, and any combination thereof.

18. (canceled)

19. The method of claim 1, wherein the cytokine is selected from the group consisting of: IL-2, an IL-2 variant, IL-7, an IL-7 variant, IL-15, an IL-15 variant, IL-18, an IL-18 variant, IL-21, an IL-21 variant, and combination thereof.

20. The method of claim 2, wherein the concentration of the cytokine added during the first time period is at least about 10 IU/mL or about 50 IU/mL.

21. (canceled)

22. The method of claim 2, wherein the concentration of the cytokine added during the second time period is at least about 4000 IU/mL, or about 6000 IU/mL.

23. (canceled)

24. (canceled)

25. The method of claim 12, wherein the time period in (d) is at least 1 day, or about 14 days.

26. (canceled)

27. The method of claim 12, wherein the one or more agents in (d) is selected from the group consisting of: a cytokine, an antibody, a modulator, and any combination thereof.

28. The method of claim 27, wherein the one or more agents further comprises irradiated feeder cells.

29. The method of claim 28, wherein the irradiated feeder cells are irradiated allogeneic PBMC-derived feeder cells.

30.-33. (canceled)

34. The method of claim 27, wherein the antibody comprises an anti-CD3 antibody.

35.-37. (canceled)

38. The method of claim 27, wherein the modulator comprises a Notch signaling pathway modulator, an interferon gamma (IFNγ) modulator, or a combination thereof.

39.-42. (canceled)

43. The method of claim 2, wherein the method further comprises providing at least one additional reagent during the first time period or the second time period, wherein the at least one additional reagent is selected from the group consisting of: PD-1, CD39, 4-1BB-positive T cells, CXCR3-binding chemokines, an interferon, a checkpoint inhibitor, a TLR agonist, a modulator of RIG-I-like receptors, a modulator of NOD-like receptors, a modulator of C-type lectin receptors, a modulator of STING, and any combination thereof.

44.-56. (canceled)

57. The method of claim 1, wherein the method further comprises reprogramming the immune cells.

58. The method of claim 57, wherein the immune cells are reprogrammed by activation of Notch signaling pathway during (d), inhibition of interferon gamma (IFNγ) signaling pathway during (d), or both.

59-62. (canceled)
